(19) United States
(12) Patent Application Publication
(10) Pub. No.: US 2023/0203147 A1
(43) Pub. Date: Jun. 29, 2023

(54) AQUEOUS FORMULATIONS OF TNF-ALPHA ANTIBODIES IN HIGH CONCENTRATIONS

(71) Applicant: ALVOTECH HF, Reykjavik (IS)

(72) Inventors: Elin EDWALD, Reykjavik (IS); Hordur K HARDARSON, Reykjavik (IS)

(73) Assignee: ALVOTECH HF, Reykjavik (IS)

(21) Appl. No.: 17/439,945

(22) PCT Filed: Mar. 13, 2020
(86) PCT No.: PCT/EP2020/056908

§ 371 Date: Sep. 16, 2021

Related U.S. Application Data
(60) Provisional application No. 62/819,823, filed on Mar. 18, 2019.

(51) Int. Cl. C07K 16/24; A61K 47/26; A61K 47/12; A61K 9/08

(52) U.S. Cl. CPC C07K 16/241; A61K 9/08; A61K 47/12; A61K 47/26

(57) ABSTRACT

The present invention relates to stable, aqueous formulations of adalimumab. Particularly, stable, aqueous formulations comprising high concentration (e.g. about 100 mg) of adalimumab, trehalose or sucrose, nonionic surfactant, low concentration to no buffer, with no ionic tonicity-adjusting agents and no amino acid stabilizers.

This specification includes 16 figures, some of which include multiple parts. FIGS. 1 to 15 are in colour.

### FIELD OF THE INVENTION

The present invention relates to stable, aqueous formulations of adalimumab. Particularly, stable, aqueous formulations comprising high concentration (e.g. about 100 mg) of adalimumab, trehalose or sucrose, nonionic surfactant, low concentration to no buffer, with no ionic tonicity-adjusting agents and no amino acid stabilizers.

### BACKGROUND OF THE INVENTION

Any discussion of the prior art throughout the specification should in no way be considered as an admission that such prior art is widely known or forms part of common general knowledge in the field.

The commercial product HumiraⓇ comprises an anti-TNFa antibody known as D2E7. D2E7 is a recombinant human lgG1 monoclonal antibody specific for human TNFα consisting of 1330 amino acids with a molecular weight of approximately 148 kilodaltons. The amino acid sequence of the light chain variable region and the amino acid sequence of the heavy chain variable region of D2E7 have been described in U.S. Pat. No. 6,090,382 (referring therein to FIGS. 1A, 1B, 2A, 2B, and SEQ ID NOs: 1 and 2), the disclosure of which is hereby incorporated by reference in its entirety. D2E7 is usually produced by recombinant DNA technology in a mammalian cell expression system, such as, for example, Chinese Hamster Ovary cells. D2E7 binds specifically to TNFα and neutralizes the biological function of TNFα by blocking its interaction with the p55 and p75 cell surface TNF receptors.

D2E7 is used to treat rheumatoid arthritis, psoriatic arthritis, ankylosing spondylitis, Crohn’s disease, ulcerative colitis, moderate to severe chronic psoriasis, and juvenile idiopathic arthritis.

Formulations of antibodies, e.g. D2E7 or an antibody biosimilar thereto, may be prepared in forms that require minimal manipulation prior to administration, which may minimize human error during administration and/or is suitable for at-home administration. For example, formulations that do not comprise the addition of water at the point of treatment, such as a glass vial comprising an aqueous formulation for single-dose administration, a pre-filled syringe comprising an aqueous formulation for single-dose administration, a pre-filled injection pen comprising an aqueous formulation, and the like.

Formulations comprising an antibody (e.g. D2E7 or an antibody biosimilar thereto) administered as part of a therapeutic regimen may be administered on more than one occasion (e.g. weekly, twice monthly, monthly) and/or administered with high antibody concentrations (e.g., 50 mg/ml, 100 mg/ml, 125 mg/ml).

There remains a need for a stable, aqueous formulation of adalimumab.

It is an object of the present invention to overcome or ameliorate at least one of the disadvantages of the prior art, or to provide a useful alternative.

### SUMMARY OF THE INVENTION

It was surprisingly found that an aqueous formulation comprising a high concentration of adalimumab with trehalose or sucrose, surfactant, and low to no buffer, exhibits stability. In particular, the formulations disclosed herein are stable under long term storage.

In a first aspect, the invention provides a stable, aqueous formulation comprising
(a) about 90 mg/ml to about 125 mg/ml adalimumab;
(b) about 200 mM to about 275 mM trehalose or about 200 mM to about 275 mM sucrose;
(c) about 0.05% to about 0.15% nonionic surfactant; and
(d) about 25 mM or less acetate buffer or about 25 mM or less succinate buffer,
wherein the formulation is essentially free of ionic tonicity-adjusting agents; wherein the formulation is essentially free of amino acid stabilizer; wherein the osmolality of the formulation is about 240 mOsm/kg to about 420 mOsm/kg; and wherein the formulation is about pH 5.0 to about pH 6.0.

In a second aspect, the invention provides an aqueous formulation comprising
(a) about 100 mg/ml adalimumab;
(b) about 250 mM trehalose;
(c) about 0.1% (w/v) polysorbate 20; and
(d) about 20 mM or less acetate buffer;
wherein the formulation is essentially free of ionic tonicity-adjusting agents; wherein the formulation is essentially free of amino acid stabilizer; and wherein the formulation is about pH 5.0 to about pH 6.0.

In a third aspect, the invention provides an aqueous formulation comprising
(a) about 100 mg/ml adalimumab;
(b) about 250 mM sucrose;
(c) about 0.1% (w/v) polysorbate 20; and
(d) about 20 mM or less acetate buffer;
wherein the formulation is essentially free of ionic tonicity-adjusting agents; wherein the formulation is essentially free of amino acid stabilizer; wherein the formulation is about pH 5.0 to about pH 6.0.

In a fourth aspect, the invention provides an aqueous formulation comprising
(a) about 100 mg/ml adalimumab;
(b) about 250 mM trehalose; and
(c) about 0.1% (w/v) polysorbate 20;
wherein the formulation is essentially free of ionic excipients; and wherein the formulation is about pH 5.0 to about pH 6.0.

In a fifth aspect, the invention provides an aqueous formulation comprising
(a) about 100 mg/ml adalimumab;
(b) about 250 mM sucrose; and
(c) about 0.1% (w/v) polysorbate 20;
wherein the formulation is essentially free of ionic excipients; and wherein the formulation is about pH 5.0 to about pH 6.0.

The aqueous formulations, which are prepared to comprise a low concentration to no buffer, which are further essentially free of ionic tonicity-adjusting agents and essentially free of amino acid stabilizers, are surprisingly stable under long term storage conditions (e.g. about 2° C. to about 8° C. for at least about 3 months, about 6 months, about 12 months, or about 24 months) or under room temperature storage conditions (e.g. about 20° C. to about 25° C. for at least about 14 days).

The aqueous formulations may be formulated for self-administration to a subject (patient) or administration by a medical professional to a subject (patient). Preferably, the aqueous formulations are formulated as a single-dose presentation including but not limited to a prefilled injection pen, a prefilled syringe, and a prefilled vial (e.g., prefilled glass vial). The aqueous formulation may also be stored in bulk in a single-use bag (e.g., a pharmaceutically acceptable plastic bag, a biopharmaceutical grade bag, and the like).

It will be appreciated that an aqueous formulation will comprise water suitable for administration to a subject (patient) such as water for injection (WFI) or similar pharmaceutically acceptable water.

### Definitions

Unless the context clearly requires otherwise, throughout the description and the claims, the words “comprise”, “comprising” and the like are to be construed in an inclusive sense as opposed to an exclusive or exhaustive sense; that is to say, in the sense of “including, but not limited to”. It will be appreciated that in the context of an aqueous formulation herein described the terms “consisting essentially of” or “consisting of”, and grammatical variations thereof, do not exclude the presence of water or a pH adjusting agent required to adjust the pH of the formulation to a target pH.

As used herein and in the appended claims, the singular form of “a”, “an”, and “the” may include the plural referents unless the context clearly dictates otherwise. It is further noted that the claims may be drafted to exclude any optional element.

As used herein the term “about” can mean within 1 or more standard deviation per the practice in the art. Alternatively, “about” can mean a range of up to 20%, up to 10%, or up to 5%. In certain embodiments, “about” can mean up to 10%. For example, in the context of an aqueous formulation herein described comprising about 100 mg/ml adalimumab, encompasses an aqueous formulation may having from 90 mg/ml to 110 mg/ml adalimumab. When particular values are provided in the specification and claims the meaning of “about” should be assumed to be within an acceptable error range for that particular value.

As used herein the term “adalimumab” encompasses the anti-TNFa antibody known as D2E7 as defmed in U.S. Pat. No. 6,090,382 and anti-TNFa antibodies that are biosimilar to D2E7 (e.g. biosimilar to D2E7 in the commercial product Humira^{Ⓡ}). The sequence of the D2E7 antibody comprised in Humira^{Ⓡ} is known in the art. See https://www.drugbank.ca/drugs/DB00051 reporting the sequence of the light chain and heavy chain of adalimumab in Humira^{Ⓡ}. See FIG. 16. It is understood that an anti-TNFα antibody biosimilar to the antibody comprised in the commercial product Humira^{Ⓡ} may be designated, according to the requirements of the United States Food and Drug Administration, as “adalimumab-xxxx”, wherein the “xxxx” indicates the origin of the adalimumab. It will be appreciated that a biosimilar adalimumab is evaluated by physiochemical and functional similarity to adalimumab comprised in a commercial product (Humira^{Ⓡ}). See Liu et al., BioDrugs (2016) 30:321-338; Magnenat et al., MABS (2017) 9(1):127-139. Antibodies biosimilar to D2E7 include but are not limited to antibodies with one or two modifications (deletion, addition, and/or substitutions of amino acids) in the amino acid sequence of D2E7 that do not significantly affect the biological function (e.g. TNFa-binding, Fc_{γ}RIIIa, or the like) of the antibody, and antibodies with a glycosylation profile that is different from D2E7 in the commercial product Humira^{Ⓡ}. The anti-TNFa antibody D2E7 in Humira^{Ⓡ} is a recombinant human 1 gG1 monoclonal antibody having two light chains, each with a molecular weight of approximately 24 kilodaltons (kDa) and two 1 gG1 heavy chains each with a molecular weight of approximately 49 kDa. Each light chain consists of 214 amino acid residues and each heavy chain consists of 451 amino acid residues. Thus, anti-TNFa antibody D2E7 in Humira^{Ⓡ} has 1330 aminoacids and has a total molecular weight of approximately 148 kDa. Biosimilar antibodies may also be referred to as bio-better candidates. It is understood in the art that a biosimilar antibody or a bio-better candidate are antibodies which biosimilar approval by a regulatory agency is sought. Preferably, adalimumab in an aqueous formulation described herein is an antibody having the same 1330 amino acid resides as the D2E7 antibody in Humira^{Ⓡ}. See FIG. 16.

The term “TNF-alpha” (which may be abbreviated as TNF_{α}), as used herein, is intended to refer to a human cytokine that exists as a 17 kD secreted form and a 26 kD membrane associated form, the biologically active form of which is composed of a trimer of noncovalently bound 17 kD molecules. The structure of TNF-alpha is described further in, for example, Permica, D., et al. (1984) Nature 312:724-729; Davis, J. M., et al. (1987) Biochemistry 26:1322-1326; and Jones, E. Y., et al. (1989) Nature 338:225-228. The term TNF-alpha is intended to include recombinant human TNF-alpha (rTNFa), which can be prepared by standard recombinant expression methods or purchased commercially.

The term “antibody”, as used herein, refers to an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

There are five types of vertebrate heavy chains: alpha, delta, epsilon, gamma, and mu. Each heavy chain is comprised of a variable region and three constant regions. The five heavy chain types define five classes of vertebrate antibodies (isotypes): IgA, IgD, IgE, IgG, and IgM. Each isotype is made up of, respectively, (a) two alpha, delta, epsilon, gamma, or mu heavy chains, and (b) two kappa or two lambda light chains. The heavy chains in each class associate with both types of light chains; but, the two light chains in a given molecule are both kappa or both lambda. IgD, IgE, and IgG generally occur as “free” heterotetrameric glycoproteins. IgA and IgM generally occur in complexes comprising several IgA or several IgM heterotetramers associated with a “J” chain polypeptide. Some vertebrate isotypes are classified into subclasses, distinguished from one another by differences in constant region sequences. There are four human IgG subclasses, IgG1, IgG2, IgG3, and lgG4, and two IgA subclasses, IgA1 and lgA2, for example. All of these and 5 others not specifically described above are included in the meaning of the term “antibody” as used herein.

As used herein, the term “buffer” refers to, for example, a weak acid and its salt, or a weak base and its salt, which when in solution is able to neutralize amounts of acid or base to maintain the pH of the solution. Exemplary buffers include, but are not limited to, acetate buffer (acetate/acetic acid), succinate buffer (succinate/succinic acid), and the like. It is well understood that a buffer may be prepared by mixing buffer components with a strong acid (e.g. HCl) or a strong base (e.g. NaOH) to adjust the buffer to a target pH and thereby a buffer may comprise a residual concentration of salt ions, e.g. chloride ions, sodium ions. The term buffer as used herein does not include the self-buffering capacity of adalimumab. As used herein any concentration of a buffer refers to the combined concentration of all the components (acid and conjugate base or, alternatively, base and conjugate acid) of the buffer in solution (e.g. for “acetate buffer” the combined concentration of acetate salt and acetic acid in the solution, for “succinate buffer” the combined concentration of succinate salt and succinic acid in the solution).

As used herein, the term “nonionic surfactant” refers to a compound comprising a hydrophilic head group and a hydrophobic tail carrying no charge. Exemplary nonionic surfactants include, but are not limited to, sorbitan fatty acid esters, polyoxyethylene sorbitan fatty acid esters, polyoxyethylene polyoxypropylene glycols, polyoxyethylene hydrogenated castor oils, polyethylene glycol fatty acid ethers, glycerine fatty acid esters, sucrose fatty acid esters and the like. Particularly suitable nonionic surfactants for inclusion in the aqueous formulations described herein include polysorbate 20 (polyoxyethylene sorbitan monolaurate), polysorbate 80 (polyoxyethylene sorbitan monooleate), and the like, at a concentration of about 0.05% (w/v) to about 0.15% (w/v). Polysorbate 20 at a concentration of about 0.1% (w/v) is particularly suited to the aqueous formulations herein described.

As used herein, “w/v” or “(w/v)” is used to denote weight (g) of solute per volume (ml) solution. “% (w/v)” is used to denote weight (g) of solute per volume (ml) of solution x 100.

A stable, aqueous formulation herein described is “essentially free of ionic tonicity-adjusting agents”. Ionic tonicity-adjusting agents are well known in the art and include inorganic salts of sodium, potassium, calcium and the like. For example, the stable, aqueous formulation herein described can be essentially free of a ionic tonicity adjusting agent such as sodium chloride (NaCl), magnesium chloride (MgC1₂), calcium chloride (CaC1₂), sodium metabisulfite (Na₂S₂O₅), and the like. A stable, aqueous formulation “essentially free of ionic tonicity adjusting agents” is, for example, formulated to remove an ionic tonicity-adjusting agent(s) that may be present during production and/or purification of adalimumab, which is to be comprised in said stable, aqueous formulation; formulated without the addition of an ionic tonicity-adjusting agent(s); and the like. It will be appreciated that methods of removing an ionic tonicity-adjusting agent(s) are not particularly limited and include, for example, well known methods such as dialysis, desalting columns, diafiltration, and the like. It will further be appreciated that such methods are known to leave a certain percentage of target agent to be removed. In the context herein, a stable aqueous formulation “essentially free of ionic tonicity-adjusting agents” includes a formulation prepared by methods to remove an ionic tonicity-adjusting agent wherein the ionic tonicity-adjusting agent is removed to the limits of a particular removal method. In certain embodiments, “essentially free of ionic-tonicity adjusting agents” refers to less than about 0.1% (w/v) of each of one or more ionic-tonicity adjusting agents. In certain embodiments, “essentially free of ionic-tonicity adjusting agents” refers to less than about 0.01% (w/v) of each of one or more ionic-tonicity adjusting agents. It will be appreciated that a stable, aqueous formulation essentially free of ionic excipients does not exclude addition of a pH adjuster, e.g. hydrochloric acid (HCl) or sodium hydroxide (NaOH) to achieve a target pH, e.g. a pH of about 5.0 to about 6.0, in the presence of buffer (acetate buffer or succinate buffer) or absence of buffer.

A stable, aqueous formulation herein described is “essentially free of amino acid stabilizers”. Amino acid stabilizers are well known in the art and include methionine, glycine, arginine, and the like. The term “amino acid stabilizer” refers to an exogenous amino acid added to a formulation and does not encompass an amino acid residue of the adalimumab or an amino acid residue derived from, e.g., degradation of adalimumab in a formulation. A stable, aqueous formulation “essentially free of amino acid stabilizers” is, for example, formulated to remove an amino acid stabilizer(s) that may be present during production and/or purification of adalimumab, which is to be comprised in said stable, aqueous formulation; formulated without the addition of an amino acid stabilizer(s); and the like. It will be appreciated that methods of removing an amino acid stabilizer are not particularly limited and include, for example, well known methods such as dialysis, desalting columns, diafiltration, and the like. In the context herein, a stable aqueous formulation “essentially free of amino acid stabilizer” includes a formulation prepared by methods to remove an amino acid stabilize wherein the amino acid stabilize is removed to the limits of a particular removal method. In certain embodiments, “essentially free of amino acid stabilizers” refers to less than about 0.1% (w/v) of each of one or more amino acid stabilizers. In certain embodiments, “essentially free of amino acid stabilizers” refers to less than about 0.01% (w/v) of each of one or more amino acid stabilizers.

A stable, aqueous formulation herein described may be “essentially free of ionic excipients”. Ionic excipients include ionic tonicity-adjusting agents, amino acid stabilizers, and buffers, each as herein defmed. A stable, aqueous formulation “essentially free of ionic excipients” is, for example, formulated to remove such ionic excipients that may be present during production and/or purification of adalimumab, which is to be comprised in said stable, aqueous formulation; formulated without the addition of an amino acid stabilizer(s); and the like. It will be appreciated that the buffering capacity of adalimumab is not excluded in a stable, aqueous formulation that is essentially free of ionic excipients. A stable, aqueous formulation that is essentially free of ionic tonicity-adjusting agents and essentially free of amino acid stabilizers formulated to remove buffer or without buffer is a stable, aqueous formulation that is essentially free of ionic excipients. It will be appreciated that the buffering capacity of adalimumab is not excluded in a stable, aqueous formulation that is essentially free of ionic excipients. It will be appreciated that methods of removing ionic excipients are not particularly limited and include, for example, well known methods such as dialysis, desalting columns, diafiltration, and the like. In the context herein, a stable aqueous formulation “essentially free of ionic excipients” includes a formulation prepared by methods to remove ionic excipients wherein the ionic excipients are removed to the limits of a particular removal method. In certain embodiments, “essentially free of ionic excipients” refers to less than about 0.1% (w/v) of each of one or more ionic excipients. In certain embodiments, “essentially free of ionic excipients” refers to less than about 0.01% (w/v) of each of one or more ionic excipients. It will be appreciated that a stable, aqueous formulation essentially free of ionic excipients does not exclude addition of a pH adjuster, e.g. hydrochloric acid (HCl) or sodium hydroxide (NaOH) to achieve a target pH, e.g. a pH of about 5.0 to about 6.0, in the presence of buffer (acetate buffer or succinate buffer) or absence of buffer.

As used herein, the term “stable” describes an aqueous formulation comprising adalimumab having a low level of degradation of adalimumab (e.g. physical degradation such as aggregation, fragmentation, deamidation, oxidation, changes in glycosylation, and/or the like, and/or biological degradation such as decrease in activity). Determination of the stability of a formulation may be assessed after exposure of the formulation to one or more stress conditions and determining whether the formulation exhibits from no detectable level to an acceptable level of perturbation. Perturbation refers to a change in the components in an aqueous formulation, particularly adalimumab, after exposure to one or more stress conditions and includes, but is not limited to, a change in one or more of visible or subvisible particles, a change in acidic species (e.g. deamidation) of adalimumab comprised in the formulation, a change in basic species of the adalimumab comprised in the formulation, oxidation of the adalimumab comprised in the formulation, a change in glycosylation of the adalimumab comprised in the formulation, a change in aggregation of the adalimumab comprised in the formulation, and/or the like. Perturbation or a level of perturbation may be determined be methods well known in the art for assessing degradation of an antibody in an aqueous formulation, including but not limited to size exclusion-high performance liquid chromatography (SEC-HPLC), cation exchange chromatography (CEX), capillary electrophoresis sodium dodecyl sulfate (CE-SDS) (non-reduced and reduced conditions), particle count light obscuration, and the like. In certain embodiments, perturbation or a level of perturbation may further include detecting a change in biological activity of adalimumab comprised in the aqueous formulation. A change in biological activity may be determined by methods well known in the art including, but not limited to, relative dissociation constant (K_{D}) surface plasmon resonance (SPR).

As used herein, the term “stress condition”, includes but is not limited to storing (storage or maintenance) of a formulation at a temperature for a given duration, e.g. about 2° C. to about 8° C. for at least about 1 month, at least about 3 months, at least about 6 months, at least about 12 months, or at least about 24 months; about 25° C. for at least about 1 month, at least about 3 months, at least about 6 months, or at least about 12 months; about 40° C. for at least about 1 month, at least about 3 months, at least about 6 months, or at least about 12 months; or exposure to conditions such as shaking, oxidation, heating, freezing, and/or mixing.

In certain embodiments, stability may be determined with reference to a control. In certain embodiments, a control may be an unstressed control not exposed to a stress condition analysed for perturbation prior to exposure to a stress condition. For example, an unstressed control may be from the same batch as the aqueous formulation undergoing stability analysis wherein the unstressed control is assessed for perturbation at time point zero (e.g. prior to storage, prior to shaking, and the like). In certain embodiments, a control may be an aqueous formulation previously determined to exhibit an acceptable level of perturbation after exposure to one or more stress conditions. In certain embodiments, a control may be a commercially available formulation, preferably Humira^{Ⓡ}, as is routinely carried out in the art. See Liu et al., BioDrugs (2016) 30:321-338; Magnenat et al., MABS (2017) 9(1):127-139. See also the methods of Example 9. A commercially available formulation may be in a presentation including, but not limited to, a single-dose prefilled injection pen, pre-filled syringe or vial.

In certain embodiments, stable may be determined with reference to a predefined level of perturbation. A predefined level of perturbation may include a percentage of aggregate, percentage of acidic species, percentage of subvisible particle. For example, a formulation may be determined to be stable after exposure to a stress condition as defmed herein and as assessed by SEC-HPLC and a relative monomer peak area of adalimumab not less than about 98%. A formulation may be determined to be stable after exposure to a stress condition as defmed herein and as assessed by CEX-HPLC and a relative acidic species peak area of adalimumab not more than about 25%. A formulation may be determined to be stable after exposure to a stress condition as defmed herein and as assessed by particle count light obscuration wherein not more than about 6000 particles of size equal to or greater than about 10 µm are detected and/or not more than about 600 particles of size equal to or greater than about 25 µm are detected. A formulation may be determined to be stable after exposure to a stress condition as defmed herein and as assessed by CE-SDS (non-reducing) and a relative “IgG” peak (intact adalimumab having two heavy chains and two light chains) not less than about 90%. A formulation may be determined to be stable after exposure to a stress condition as defmed herein as assessed by CE-SDS (reducing) and a relative heavy chain (HC) peak of about 60% to about 72% and/or a relative light chain (LC) peak of about 30% to about 36%.

In certain embodiments, an aqueous formulation is considered stable when exposure to one or more stress conditions exhibits less perturbation, or no more perturbation, in comparison to a control. In related embodiments, an aqueous formulation is considered stable when exposure to one or more stress conditions exhibits less perturbation, or no more perturbation, in comparison to a control exposed to the same one or more stress conditions. In related embodiments, an aqueous formulation is considered stable when exposure to one or more stress conditions exhibits less perturbation, or no more perturbation, in comparison to an unstressed control.

As used herein, the term “acceptable level” with reference to a level of perturbation in an aqueous formulation includes a level that would not render the aqueous formulation unsuitable for its intended pharmaceutical application. For example, an acceptable level with regard to a level of perturbation for an aqueous formulation includes, but is not limited to, no more than or less perturbation than is present in a commercially available formulation exposed to the same stress condition or no more than or less perturbation than is present in a commercially available formulation without exposure to any stress condition. In certain embodiments, as acceptable level of perturbation as assessed by SEC-HPLC wherein a relative monomer peak area of adalimumab not less than about 98%; as assessed by CEX-HPLC wherein a relative acidic species peak area of adalimumab not more than about 25%; as assessed by particle count light obscuration wherein not more than about 6000 particles of size equal to or greater than about 10 µm are detected and/or not more than about 600 particles of size equal to or greater than about 25 µm are detected; as assessed by CE-SDS (non-reducing) wherein a relative “IgG” peak (intact adalimumab having two heavy chains and two light chains) not less than about 90%; and/or as assessed by CE-SDS (reducing) and a relative heavy chain (HC) peak of about 60% to about 72% and/or a relative light chain (LC) peak of about 30% to about 36%.

As used herein the term “long term storage” includes, but is not limited to, about 2° C. to about 8° C. for at least about 3 months, at least about 6 months, at least about 12 months, and/or at least about 24 months.

As used herein, the term “room temperature storage” includes, but is not limited to, about 20° C. to about 25° C. for about 14 days.

### BRIEF DESCRIPTION OF THE FIGURES

Embodiments of the invention will now be described, by way of example only, with reference to the accompanying drawings as follows.

FIGS. 1A to 1F: Melting temperatures of adalimumab in the presence of various concentrations and species of inorganic salt. See Example 1. A: Bar chart of all melting temperatures determined for the main peak. Each value is an average of four determined melting temperatures and errors bars are the standard deviations as determined by the STDEV.S function in Microsoft Excel. Left to right: 5 mM, 25 mM, 125 mM MgC1₂, 5 mM, 25 mM, 125 mM NaCl, 5 mM, 25 mM, 125 mM Na₂S₂O₅, 5 mM, 25 mM, 125 mM KCl, 5 mM, 25 mM, 125 mM CaC1₂, No Salt (WFI). B to F: Melting curves for each species of salt tested: MgC1₂ (B), NaCl (C), KC1 (D), Na₂S₂O₅ (E), CaC1₂ (F). In some cases, a second population or shoulder appears in the melting curves at lower temperatures. The two peaks are referred to as Tm High (the main peak) and Tm Low (shoulder or second population). Both are listed in Table 1 for each sample. Each curve is an average curve of four melting curves collected for each sample.

FIGS. 2A to 2F: Melting temperatures of adalimumab in the presence of various concentrations and species of sugars/polyol. See Example 2. A: Bar chart of all melting temperatures determined for the main peak (Tm). Each value is an average of four determined melting temperatures and errors bars are the standard deviations as determined by the STDEV.S function in Microsoft Excel. Left to right: 5 mM, 25 mM, 125 mM mannitol, 5 mM, 25 mM, 125 mM sorbitol, 5 mM, 25 mM, 125 mM sucrose, 5 mM, 25 mM, 125 mM trehalose, 5 mM, 25 mM, 125 mM glycerol, no sugar/polyol. B to F: Melting curves for each species of sugar/polyol tested: mannitol (B), sorbitol (C), sucrose (D), trehalose (E), glycerol (F). Each curve is an average curve of four melting curves collected for each sample. In these cases, no second population or shoulder appears in the melting curves at lower temperatures. All melting temperatures are listed in Table 2.

FIGS. 3A to 3F: Melting temperatures of adalimumab in the presence of various buffers at various pH values. See Example 3. A: Graph of melting temperatures as a function of the pHs tested: citrate (B), acetate (C), phosphate-citrate (D), tris (E), succinate (F). The melting temperatures are averages of four values determined for each sample’s main peak. The error bars are fixed at ±0.1 pH unit on the x- axis and are standard deviations of the four values on the y-axis as determined by the STDEV.S function in Microsoft Excel. The trendline is a 4-factor polynomial and is only intended to guide the eye. B to F: Melting curves of each pH and buffer condition tested. Each curve is an average of the four curves collected for each sample. In some cases (at lower pH values), a second population or shoulder appears in the melting curves at lower temperatures. Only one melting temperature was determined for each curve because determining the main peak (Tm) was sufficient to reveal the clear pH dependence of the thermal stability of adalimumab, which was the aim of this portion of the experiment. Determined Tm values are listed in Table 3.

FIG. 4: Turbidity (absorbance at 320 nm) measurements after 4 days of shaking at 300 rpm at variable pH and buffer conditions. See Example 4. Each value is an average of three measurements. The error bars are the standard deviations as determined by the STDEV.S function in Microsoft Excel on the x-axis, and 0.1 pH unit on the x-axis. The no-buffer condition (WFI), while not plotted here, yielded an O.D. of 0.4 in this case. The trendline is linear and is only intended to guide the eye.

FIGS. 5A to 5E: Buffer-concentration dependence of the adalimumab melting temperatures in the presence of four buffers at a fixed pH of 5.2. See Example 5. A: Bar graph of melting temperatures of adalimumab in four buffers at variable concentrations. Each value is an average of four melting point determinations. As for the previous buffer experiment (FIG. 4), only the main peak was determined. Error bars are standard deviations as determined by the STDEV.S function in Microsoft Excel. B to E: Melting curves of each pH and buffer condition tested: acetate (B), succinate (C), citrate (D), phosphate-citrate (E). Each curve is an average of the four curves collected for each sample. Determined Tms are listed in Table 4.

FIG. 6: The effect of polysorbate on the agitative stability of adalimumab. See Example 6. The bar chart depicts turbidity (absorbance at 320 nm) measurements after 4 days of shaking at 300 rpm in the presence or absence of 0.1% (w/v) of polysorbate 20 and polysorbate 80. The polysorbate conditions were executed and measured three times and the error bars represents the standard deviation as determined by STDEV.S in Microsoft Excel.

FIGS. 7A to 7B: The effect of L-Arginine on the stability of adalimumab. See Example 7. A: Melting curve of adalimumab in the presence of 50 or 200 mM L-Arginine. Each melting curve is an average of four curves. B: Depicts turbidity (absorbance at 320 nm) measurements after a 4 day shaking experiment. The 200 mM L-Arginine condition was executed and measured three times and the error bar represents the standard deviation as determined by STDEV.S in Microsoft Excel.

FIGS. 8A to 8B: Titrations of adalimumab in water with HCl (Orange line - about pH 7 to about pH 4, represented as negative µl values) and NaOH (Blue line - about pH 7 to about pH 9). See Example 8. A: Titration conducted at 108 mg/ml adalimumab. B: Titration conducted at 1 mg/ml adalimumab. Each starting volume was 5 ml and HCl and NaOH of either 0.2 or 0.002 M concentration as indicated were added 50 µl at a time.

FIGS. 9A to 9C: SEC-HPLC aggregate determination. A: Relative monomer peak for 2° C. to 8° C. measured at 0, 1, 3, 6 and 12 months. B: Relative monomer peak for 25° C. measured at 0, 1, 3, and 6. C: Relative monomer peak for 40° C. measured at 0, 1, and 3 months. See Example 9.

FIGS. 10A to 10C: MFI subvisible particle determination. A: Total particle concentration (particle/ml) at 2° C. to 8° C. measured at 0, 3, 6, and 12 months. B: Total particle concentration (particle/ml) at 25° C. measured at 0, 3, and 6 months. C: Total particle concentration (particle/ml) at 40° C. measured at 0 and 3 months. See Example 9.

FIGS. 11A to 11F: Charged species determination by CEX-HPLC. A: Relative main peak area at 2° C. to 8° C. measured at 0, 1, 3, 6 and 12 months. B: Relative main peak area at 25° C. measured at 0, 1, 3 and 6 months. C: Relative acidic species peak at 2° C. to 8° C. measured at 0, 1, 3, 6 and 12 months. D: Relative acidic species peak area at 25° C. measured at 0, 1, 3 and 6 months. E: Relative basic species peak at 2° C. to 8° C. measured at 0, 1, 3, 6 and 12 months. F: Relative basic species peak area at 25° C. measured at 0, 1, 3 and 6 months. See Example 9.

FIGS. 12A to 12F: Size variant determination by CE-SDS (non-reducing) and CE-SDS (reducing). A: Relative IgG peak area at 2° C. to 8° C. by Non-reducing CE-SDS measured at 0, 1, 3, 6, and 12 months. B: Relative IgG peak area at 25° C. by Non-reducing CE-SDS measured at 0, 1, 3, and 6 months. C: Relative IgG peak area at 40° C. by Non-reducing CE-SDS measured at 0, 1, and 3 months. D: Relative IgG peak area at 2° C. to 8° C. by Reducing CE-SDS measured at 0, 1, 3, 6, and 12 months. E: Relative IgG peak area at 25° C. by Reducing CE-SDS measured at 0, 1, 3, and 6 months. F: Relative IgG peak area at 40° C. by Reducing CE-SDS measured at 0, 1, and 3 months.

FIG. 13: Osmolality (mOsm/kg) for the formulations measured at time point 0, 6 months (2° C. to 8° C.) and 6 months (25° C.). In the figure in each set of three bars, the time points from left to right are 0, 6 months (2° C. to 8° C.) and 6 months (25° C.). See Example 9.

FIG. 14: Relative mean KD by TNF-alpha SPR at 2° C. to 8° C. measured at 0, 1, 3, 6 and 12 months. See Example 9.

FIG. 15: Relative mean KD by FcγRIIIa SPR at 2° C. to 8° C. measured at 0, 4, and 12 weeks. See Example 9.

FIG. 16: The amino acid sequence of the light chain and heavy chain of Adalimumab comprised in the commercial product Humira^{Ⓡ} as described in https://www.drugbank.ca/drugs/DB00051.

### DETAILED DESCRIPTION OF PREFERRED EMBODIMENTS

Pre-formulation studies of an antibody may provide information with regard to the ability of a given component to stabilize or alternatively destabilize an antibody formulation against stresses, e.g. thermal stress, agitative stress. However, such pre-formulation studies are not entirely definitive with regard to the stabilizing or destabilizing influence of a given component nor do individual pre-formulation studies capture stabilizing or destabilizing impact of a combination of components. Conflicting information may be generated in pre-formulation studies.

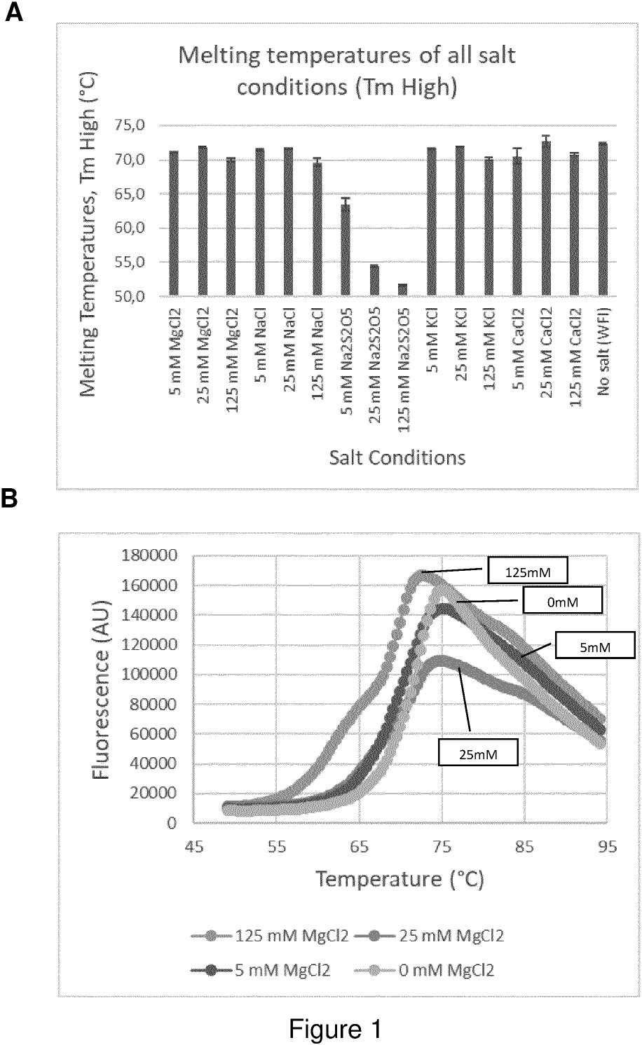

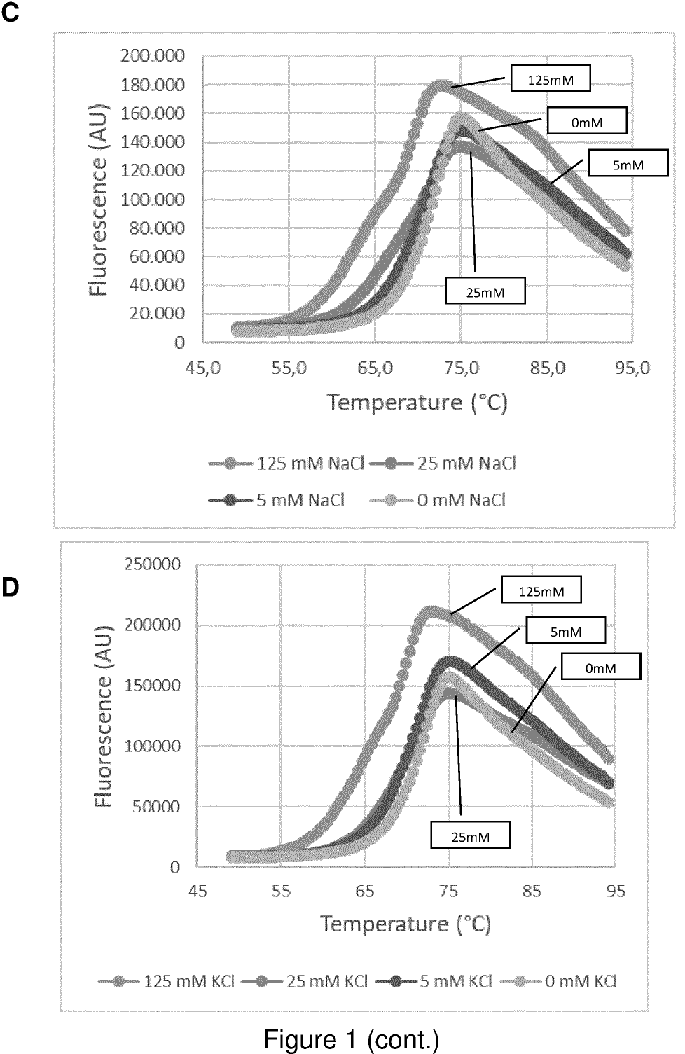

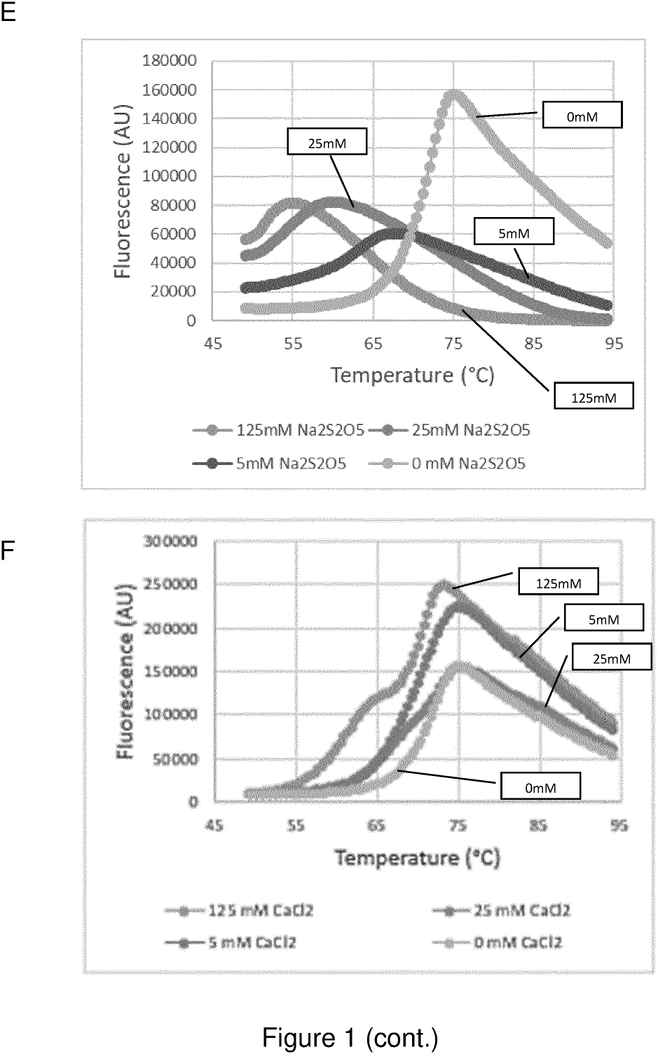

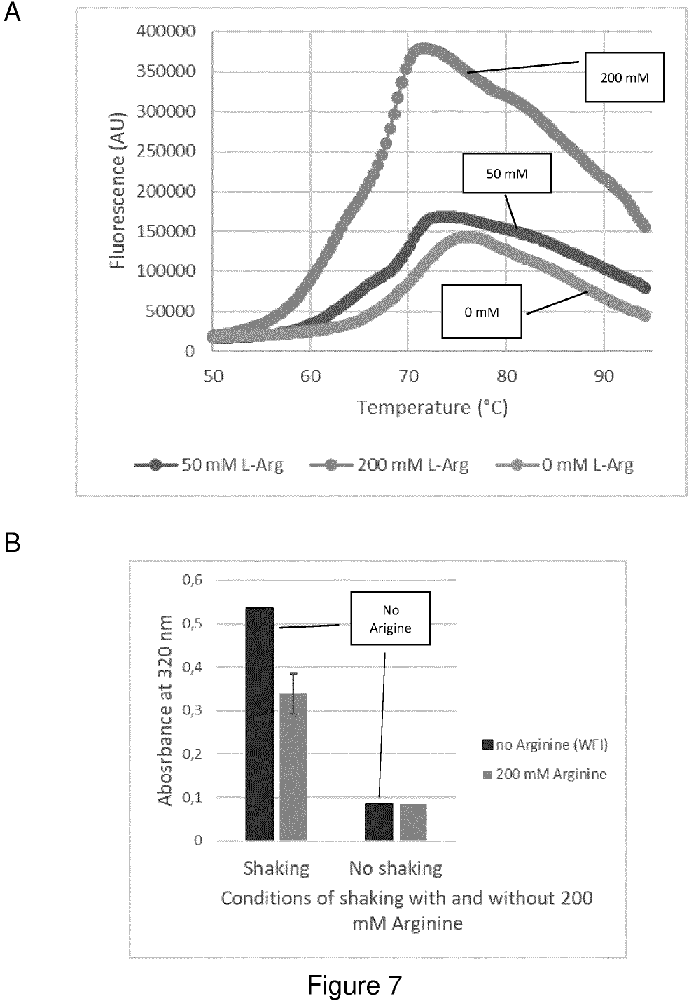

Pre-formulation studies focusing on thermal stability suggested destabilizing potential of ionic species such as inorganic salt and buffer on the stability of adalimumab. See Examples 1, 4 and 5 and FIGS. 1, 3, and 5. However, pre-formulation studies focusing on agitative stress (shaking) indicated L-arginine has a stabilizing effect. See Example 7 and FIG. 7.

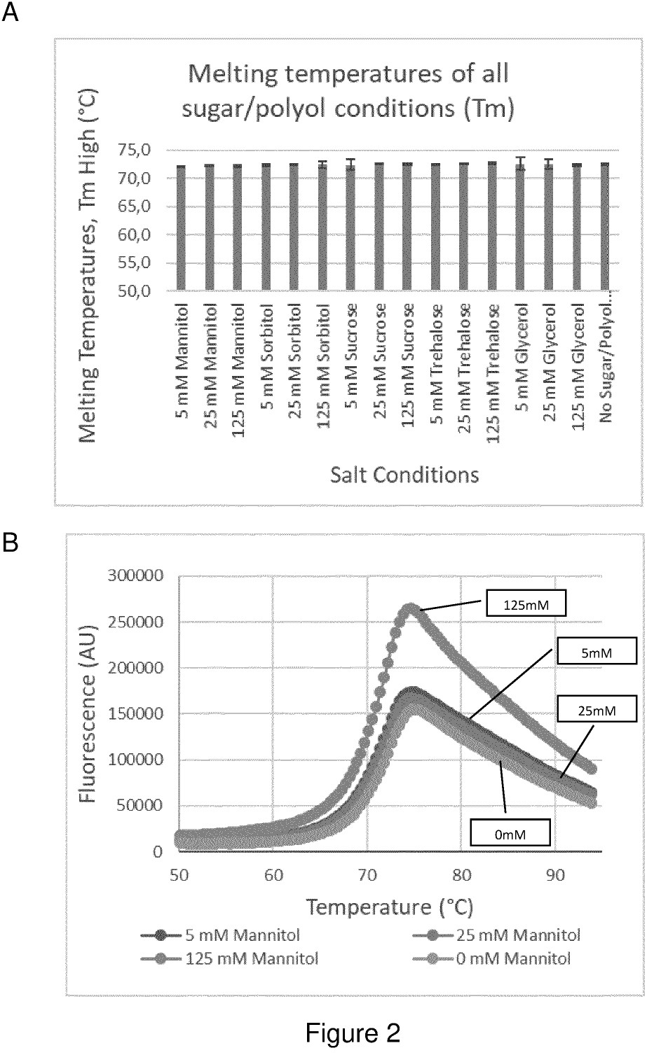

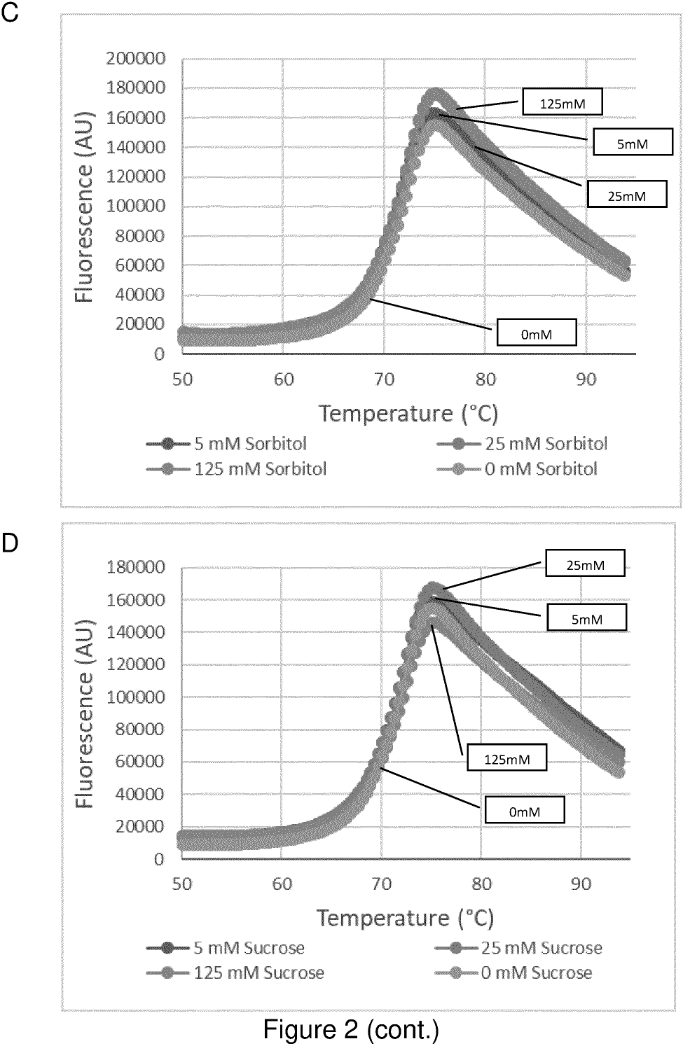

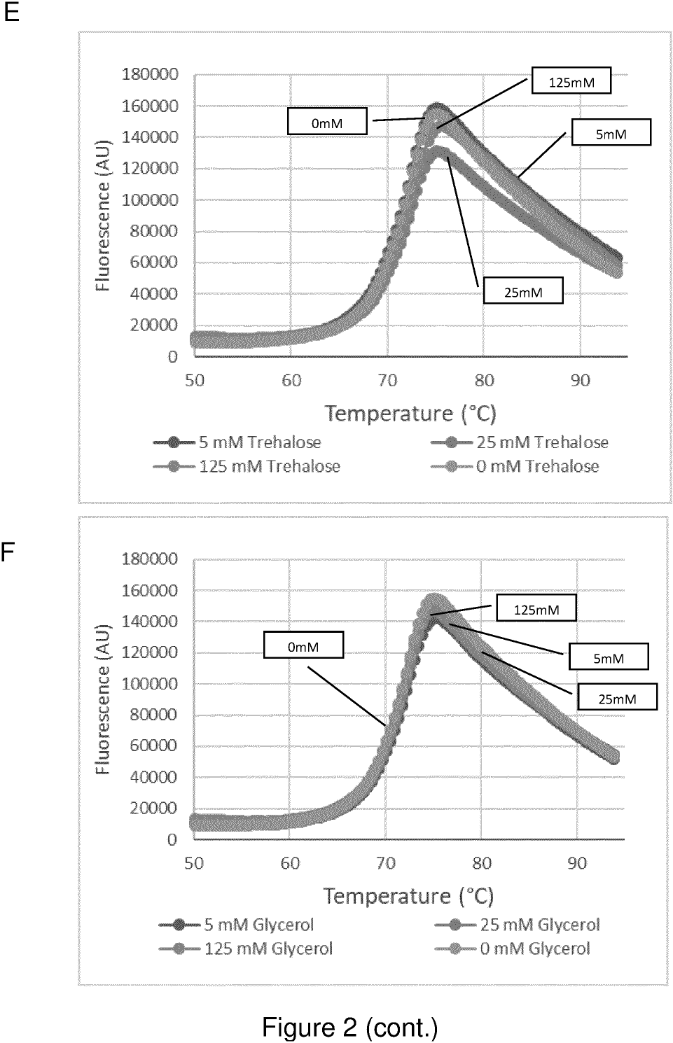

Pre-formulation studies suggested suitability of adalimumab formulations to sugars such as trehalose and sucrose and indicated a potential for increased stability with trehalose formulations. See Example 2 and FIG. 2.

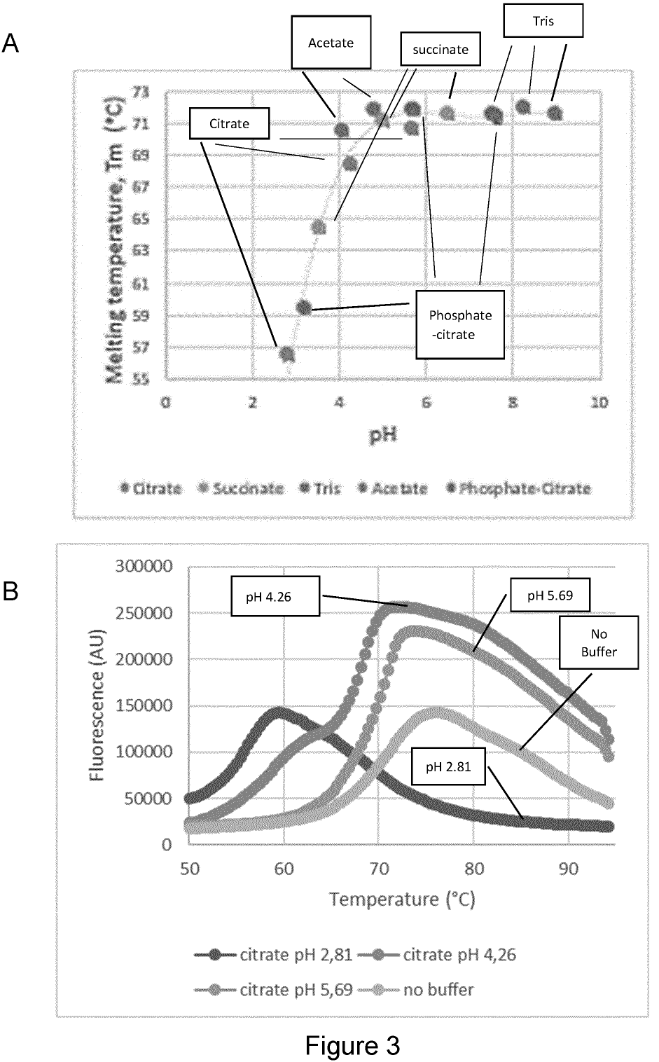

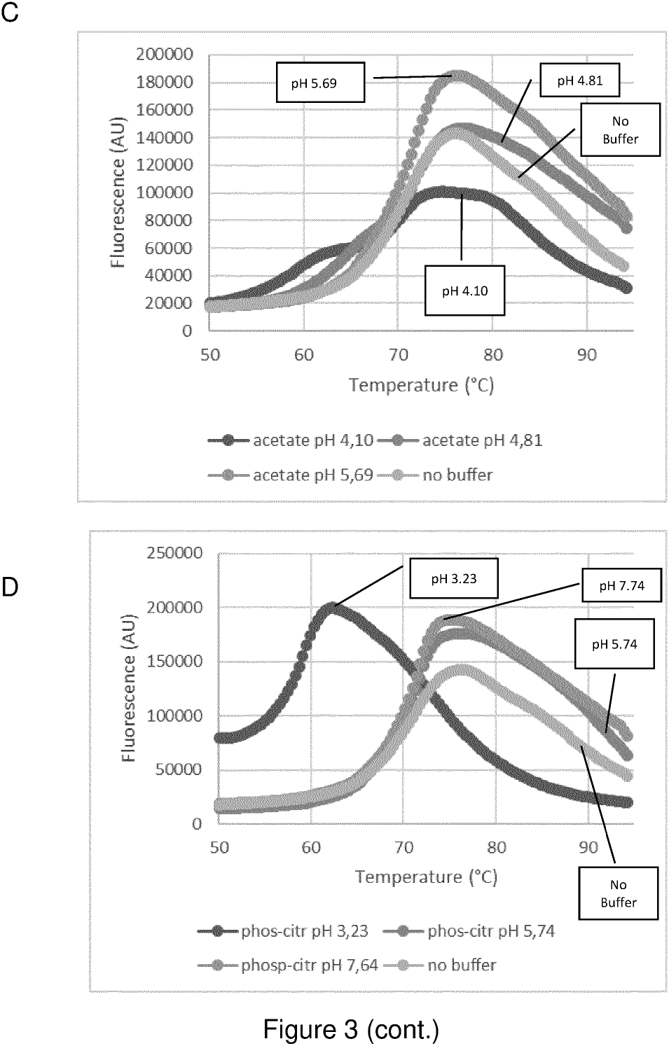

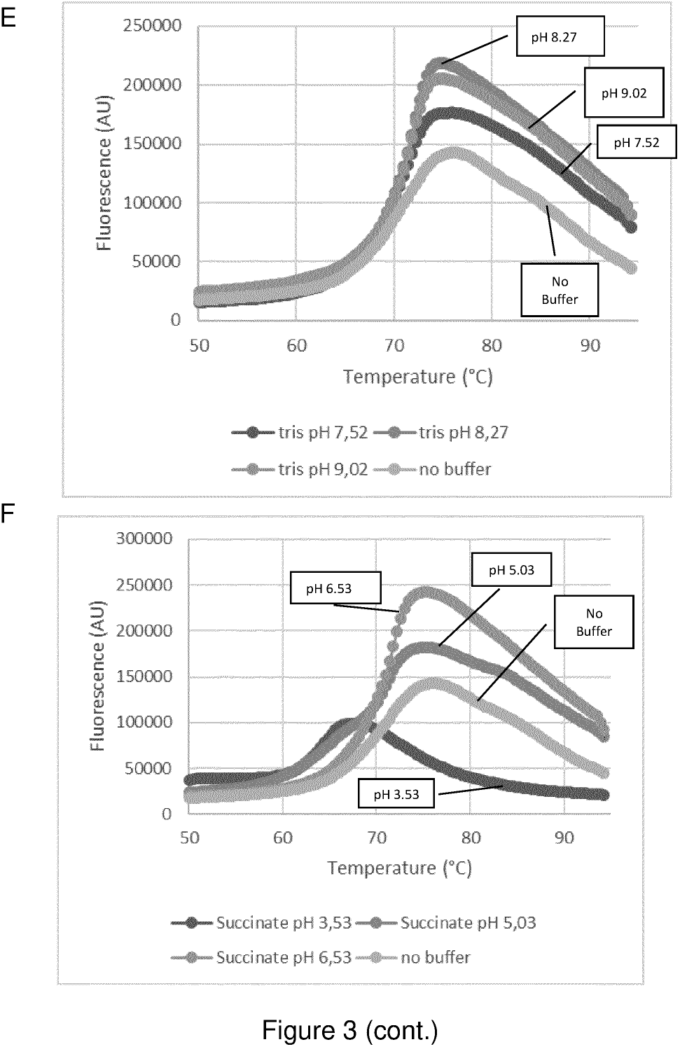

Pre-formulation studies focusing on thermal melting suggested adalimumab was sensitive to pH, with a trend of increasing stability with increasing pH, particularly from about pH 6 and higher. See Example 3 and FIG. 3.

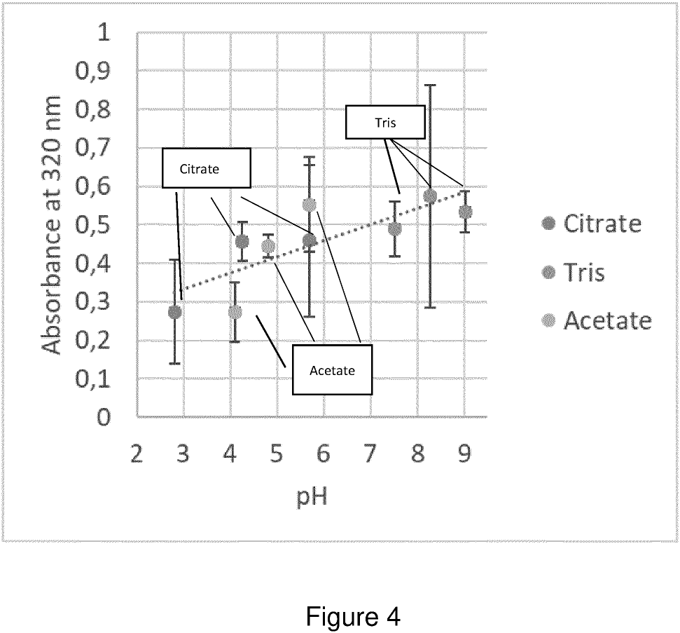

Pre-formulation studies focusing on agitative stress (shaking) indicated stabilizing potential of low pH. See Example 4, FIG. 4. This observation was opposite of what was observed in Example 3, FIG. 3, where higher pH was stabilizing.

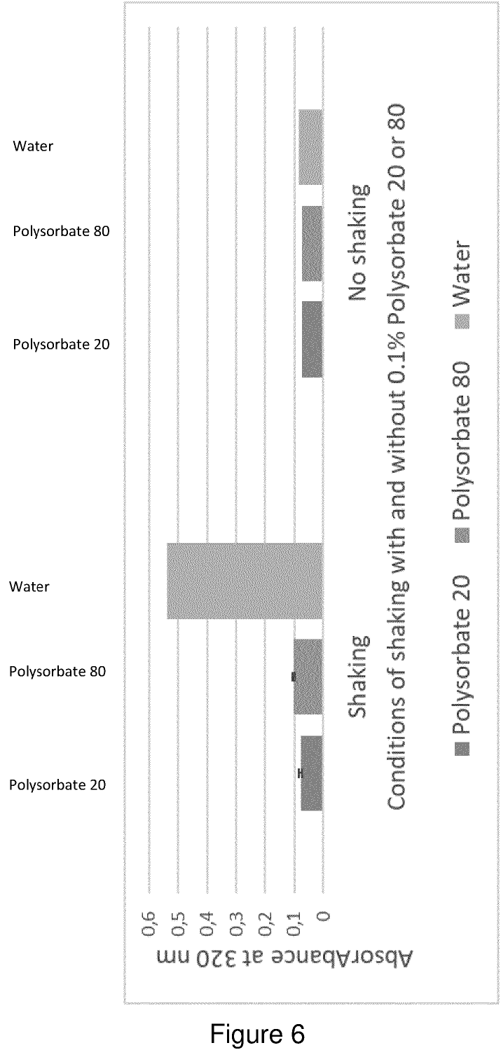

Pre-formulation studies focusing on agitative stress (shaking) indicated stabilizing potential of polysorbate 80 and polysorbate 20, with a trend toward enhanced stability of formulation comprising polysorbate 20. See Example 6 and FIG. 6.

While general trends can be observed in pre-formulation testing, such studies may provide conflicting trends. Thus, pre-formulation studies alone may generally not allow for firm conclusions as to the stability of formulations under long term storage (e.g. formulations stored at about 2° C. to about 8° C. for about 3 months, about 6 months, about 12 months, about 24 months, or more). Likewise, pre-formulation studies looking to the stabilizing/destabilizing trend for, e.g. a single type of excipient, may generally not allow for firm conclusions as to the stability of formulations under long term storage comprising multiple types of excipients.

Ultimately the stability of any formulation will be evident in analysis of formulations studied after longer duration studies, for example analysis after at least 3 months, 6 months, or longer.

It was surprisingly found that adalimumab showed high stability in the absence of excipients. It was found that the addition of most excipients was generally destabilizing, with either little positive effect on stability or destabilizing. It was discovered that a few excipients, e.g. trehalose, sucrose, were well tolerated, allowing for the use of such excipients. It was surprising found that aqueous formulations of adalimumab comprising high concentrations of adalimumab (e.g. at least about 100 mg/ml) in trehalose or sucrose were stable when the formulations comprised a about 0.1% (w/v) polysorbate 20, low to no buffer at pH of about 5 to about 6 and no other ionic excipients were added to the formulation (specifically no ionic tonicity adjusting agents and no amino acid stabilizers).

In one embodiment, provided is a stable, aqueous formulation comprising:
(a) about 90 mg/ml to about 125 mg/ml adalimumab;
(b) about 200 mM to about 275 mM trehalose or about 200 mM to about 275 mM sucrose;
(c) about 0.05% (w/v) to about 0.15% (w/v) nonionic surfactant; and
(d) about 25 mM or less acetate buffer or about 25 mM or less succinate buffer,
wherein the formulation is essentially free of ionic tonicity-adjusting agents; wherein the formulation is essentially free of amino acid stabilizer; wherein the osmolality of the formulation is about 240 mOsm/kg to about 420 mOsm/kg; and wherein the formulation is about pH 5.0 to about pH 6.0. A pH of about 5.5 is particularly suited to an aqueous formulation herein described. In a related embodiment, the stable, aqueous formulation comprises about 20 mM or less acetate buffer. In a further related embodiment, the stable, aqueous formulation comprises about 20 mM acetate buffer and about 250 mM trehalose. In a further related embodiment, the stable, aqueous formulation comprises about 20 mM acetate buffer and about 250 mM sucrose. In an alternative related embodiment, the stable, aqueous formulation comprises about 20 mM or less succinate buffer. In a further related embodiment, the stable, aqueous formulation comprises about 20 mM succinate buffer and about 250 mM trehalose. In a further related embodiment, the stable, aqueous formulation comprises about 20 mM succinate buffer and about 250 mM sucrose. In a related embodiment, stability of an aqueous formulation may be determined after storing the formulation at about 2° C. to about 8° C. for at least about 3 months. In a related embodiment, stability of an aqueous formulation may be determined after storing the formulation at about 25° C. for at least about 1 month. In certain embodiments, stability is determined by analysis of a formulation by SEC-HPLC and a predefined level of perturbation. In certain embodiments, a predefined level of perturbation is a relative monomer peak area of adalimumab not less than about 98%. In certain embodiments, stability is determined by analysis of a formulation by CEX-HPLC and a predefined level of perturbation. In certain embodiments, a predefined level of perturbation is a relative acidic species peak area is not more than about 25%. In certain embodiments stability is determined by analysis of a formulation by particle count light obscuration and a predefined level of perturbation. In certain embodiments, a predetermined level of perturbation is not more than about 6000 particles of size equal to or greater than about 10 µm are detected and/or not more than about 600 particles of size equal to or greater than about 25 µm are detected. In certain embodiments, stability is determined by analysis of a formulation by CE-SDS (non-reducing) and a predefined level of perturbation. In certain embodiments, a predefined level of perturbation is a relative “IgG” peak (intact adalimumab having two heavy chains and two light chains) not less than about 90%. In certain embodiments, stability is determined by analysis of a formulation by CE-SDS (reducing) and a predefined level of perturbation. In certain embodiments, a predefined level of perturbation is a relative heavy chain (HC) peak of about 60% to about 72% and/or a relative light chain (LC) peak of about 30% to about 36%.

In one embodiment, provided is a stable, aqueous formulation comprising:
(a) about 100 mg/ml adalimumab;
(b) about 200 mM to about 275 mM trehalose or about 200 mM to about 275 mM sucrose;
(c) about 0.1% (w/v) polysorbate 20; and
(d) about 25 mM or less acetate buffer,
wherein the formulation is essentially free of ionic tonicity-adjusting agents; wherein the formulation is essentially free of amino acid stabilizer; wherein the osmolality of the formulation is about 240 mOsm/kg to about 420 mOsm/kg; and wherein the formulation is about pH 5.0 to about pH 6.0. A pH of about 5.5 is particularly suited to an aqueous formulation herein described. In a related embodiment, the stable, aqueous formulation comprising about 250 mM trehalose and about 20 mM acetate buffer. In an alternative related embodiment, the stable, aqueous formulation comprises about 250 mM sucrose and about 20 mM acetate buffer. In a related embodiment, stability of an aqueous formulation may be determined after storing the formulation at about 2° C. to about 8° C. for at least about 3 months. In a related embodiment, stability of an aqueous formulation may be determined after storing the formulation at about 25° C. for at least about 1 month. In certain embodiments, stability is determined by analysis of a formulation by SEC-HPLC and a predefined level of perturbation. In certain embodiments, a predefined level of perturbation is a relative monomer peak area of adalimumab not less than about 98%. In certain embodiments, stability is determined by analysis of a formulation by CEX-HPLC and a predefined level of perturbation. In certain embodiments, a predefined level of perturbation is a relative acidic species peak area is not more than about 25%. In certain embodiments stability is determined by analysis of a formulation by particle count light obscuration and a predefined level of perturbation. In certain embodiments, a predetermined level of perturbation is not more than about 6000 particles of size equal to or greater than about 10 µm are detected and/or not more than about 600 particles of size equal to or greater than about 25 µm are detected. In certain embodiments, stability is determined by analysis of a formulation by CE-SDS (non-reducing) and a predefined level of perturbation. In certain embodiments, a predefined level of perturbation is a relative “IgG” peak (intact adalimumab having two heavy chains and two light chains) not less than about 90%. In certain embodiments, stability is determined by analysis of a formulation by CE-SDS (reducing) and a predefined level of perturbation. In certain embodiments, a predefined level of perturbation is a relative heavy chain (HC) peak of about 60% to about 72% and/or a relative light chain (LC) peak of about 30% to about 36%.

In one embodiment, provided is a stable, aqueous formulation comprising:
(a) about 100 mg/ml adalimumab;
(b) about 200 mM to about 275 mM trehalose or about 200 mM to about 275 mM sucrose;
(c) about 0.1% (w/v) polysorbate 20; and
(d) about 25 mM or less succinate buffer,
wherein the formulation is essentially free of ionic tonicity-adjusting agents; wherein the formulation is essentially free of amino acid stabilizer; wherein the osmolality of the formulation is about 240 mOsm/kg to about 420 mOsm/kg; and wherein the formulation is about pH 5.0 to about pH 6.0. A pH of about 5.5 is particularly suited to an aqueous formulation herein described. In a related embodiment, the stable, aqueous formulation comprising about 250 mM trehalose and about 20 mM succinate buffer. In an alternative related embodiment, the stable, aqueous formulation comprises about 250 mM sucrose and about 20 mM succinate buffer. In a related embodiment, stability of an aqueous formulation may be determined after storing the formulation at about 2° C. to about 8° C. for at least about 3 months. In a related embodiment, stability of an aqueous formulation may be determined after storing the formulation at about 25° C. for at least about 1 month. In certain embodiments, stability is determined by analysis of a formulation by SEC-HPLC and a predefined level of perturbation. In certain embodiments, a predefined level of perturbation is a relative monomer peak area of adalimumab not less than about 98%. In certain embodiments, stability is determined by analysis of a formulation by CEX-HPLC and a predefined level of perturbation. In certain embodiments, a predefined level of perturbation is a relative acidic species peak area is not more than about 25%. In certain embodiments stability is determined by analysis of a formulation by particle count light obscuration and a predefined level of perturbation. In certain embodiments, a predetermined level of perturbation is not more than about 6000 particles of size equal to or greater than about 10 µm are detected and/or not more than about 600 particles of size equal to or greater than about 25 µm are detected. In certain embodiments, stability is determined by analysis of a formulation by by CE-SDS (non-reducing) and a predefined level of perturbation. In certain embodiments, a predefined level of perturbation is a relative “IgG” peak (intact adalimumab having two heavy chains and two light chains) not less than about 90%. In certain embodiments, stability is determined by analysis of a formulation by CE-SDS (reducing) and a predefined level of perturbation. In certain embodiments, a predefined level of perturbation is a relative heavy chain (HC) peak of about 60% to about 72% and/or a relative light chain (LC) peak of about 30% to about 36%.

In one embodiment, provided is a stable, aqueous formulation comprising:
(a) about 100 mg/ml adalimumab;
(b) about 200 mM to about 275 mM trehalose;
(c) about 0.1% (w/v) polysorbate 20; and
(d) about 20 mM or less acetate buffer;
wherein the formulation is essentially free of ionic tonicity-adjusting agents; wherein the formulation is essentially free of amino acid stabilizer; and wherein the formulation is about pH 5.0 to about pH 6.0. A pH of about 5.5 is particularly suited to an aqueous formulation herein described. In a related embodiment, the stable aqueous formulation is stable under storage conditions of about 2° C. to about 8° C. for at least 6 months. In a related embodiment, the stable, aqueous formulation is stable under storage conditions of about 25° C. for at least about 14 days. In a related embodiment, the stable, aqueous formulation comprises 250 mM trehalose and 20 mM acetate buffer. In a related embodiment, stability of an aqueous formulation may be determined after storing the formulation at about 2° C. to about 8° C. for at least about 3 months. In a related embodiment, stability of an aqueous formulation may be determined after storing the formulation at about 25° C. for at least about 1 month. In certain embodiments, stability is determined by analysis of a formulation by SEC-HPLC and a predefined level of perturbation. In certain embodiments, a predefined level of perturbation is a relative monomer peak area of adalimumab not less than about 98%. In certain embodiments, stability is determined by analysis of a formulation by CEX-HPLC and a predefined level of perturbation. In certain embodiments, a predefined level of perturbation is a relative acidic species peak area is not more than about 25%. In certain embodiments stability is determined by analysis of a formulation by particle count light obscuration and a predefined level of perturbation. In certain embodiments, a predetermined level of perturbation is not more than about 6000 particles of size equal to or greater than about 10 µm are detected and/or not more than about 600 particles of size equal to or greater than about 25 µm are detected. In certain embodiments, stability is determined by analysis of a formulation by by CE-SDS (non-reducing) and a predefined level of perturbation. In certain embodiments, a predefined level of perturbation is a relative “IgG” peak (intact adalimumab having two heavy chains and two light chains) not less than about 90%. In certain embodiments, stability is determined by analysis of a formulation by CE-SDS (reducing) and a predefined level of perturbation. In certain embodiments, a predefined level of perturbation is a relative heavy chain (HC) peak of about 60% to about 72% and/or a relative light chain (LC) peak of about 30% to about 36%.

In one embodiment, provided is a stable, aqueous formulation comprising:
(a) about 100 mg/ml adalimumab;
(b) about 200 mM to about 275 mM sucrose;
(c) about 0.1% (w/v) polysorbate 20; and
(d) about 20 mM or less acetate buffer;
wherein the formulation is essentially free of ionic tonicity-adjusting agents; wherein the formulation is essentially free of amino acid stabilizer; and wherein the formulation is about pH 5.0 to about pH 6.0. A pH of about 5.5 is particularly suited to an aqueous formulation herein described. In a related embodiment, the stable aqueous formulation is stable under storage conditions of about 2° C. to about 8° C. for at least 6 months. In a related embodiment, the stable, aqueous formulation is stable under storage conditions of about 25° C. for at least about 14 days. In a related embodiment, the stable, aqueous formulation comprises 250 mM sucrose and 20 mM acetate buffer. In a related embodiment, stability of an aqueous formulation may be determined after storing the formulation at about 2° C. to about 8° C. for at least about 3 months. In a related embodiment, stability of an aqueous formulation may be determined after storing the formulation at about 25° C. for at least about 1 month. In certain embodiments, stability is determined by analysis of a formulation by SEC-HPLC and a predefined level of perturbation. In certain embodiments, a predefined level of perturbation is a relative monomer peak area of adalimumab not less than about 98%. In certain embodiments, stability is determined by analysis of a formulation by CEX-HPLC and a predefined level of perturbation. In certain embodiments, a predefined level of perturbation is a relative acidic species peak area is not more than about 25%. In certain embodiments stability is determined by analysis of a formulation by particle count light obscuration and a predefined level of perturbation. In certain embodiments, a predetermined level of perturbation is not more than about 6000 particles of size equal to or greater than about 10 µm are detected and/or not more than about 600 particles of size equal to or greater than about 25 µm are detected. In certain embodiments, stability is determined by analysis of a formulation by by CE-SDS (non-reducing) and a predefined level of perturbation. In certain embodiments, a predefined level of perturbation is a relative “IgG” peak (intact adalimumab having two heavy chains and two light chains) not less than about 90%. In certain embodiments, stability is determined by analysis of a formulation by CE-SDS (reducing) and a predefined level of perturbation. In certain embodiments, a predefined level of perturbation is a relative heavy chain (HC) peak of about 60% to about 72% and/or a relative light chain (LC) peak of about 30% to about 36%.

In one embodiment, provided is a stable, aqueous formulation comprising:
(a) about 100 mg/ml adalimumab;
(b) about 200 mM to about 275 mM trehalose; and
(c) about 0.1% (w/v) polysorbate 20;
wherein the formulation is essentially free of ionic excipients; and wherein the formulation is about pH 5.0 to about pH 6.0. A pH of about 5.5 is particularly suited to an aqueous formulation herein described. In a related embodiment, the stable aqueous formulation is stable under storage conditions of about 2° C. to about 8° C. for at least 6 months. In a related embodiment, the stable, aqueous formulation is stable under storage conditions of about 25° C. for at least about 14 days. In a related embodiment, the stable, aqueous formulation comprises 250 mM trehalose. In a related embodiment, stability of an aqueous formulation may be determined after storing the formulation at about 2° C. to about 8° C. for at least about 3 months. In a related embodiment, stability of an aqueous formulation may be determined after storing the formulation at about 25° C. for at least about 1 month. In certain embodiments, stability is determined by analysis of a formulation by SEC-HPLC and a predefined level of perturbation. In certain embodiments, a predefined level of perturbation is a relative monomer peak area of adalimumab not less than about 98%. In certain embodiments, stability is determined by analysis of a formulation by CEX-HPLC and a predefinedlevel of perturbation. In certain embodiments, a predefined level of perturbation is a relative acidic species peak area is not more than about 25%. In certain embodiments stability is determined by analysis of a formulation by particle count light obscuration and a predefined level of perturbation. In certain embodiments, a predetermined level of perturbation is not more than about 6000 particles of size equal to or greater than about 10 µm are detected and/or not more than about 600 particles of size equal to or greater than about 25 µm are detected. In certain embodiments, stability is determined by analysis of a formulation by by CE-SDS (non-reducing) and a predefined level of perturbation. In certain embodiments, a predefined level of perturbation is a relative “IgG” peak (intact adalimumab having two heavy chains and two light chains) not less than about 90%. In certain embodiments, stability is determined by analysis of a formulation by CE-SDS (reducing) and a predefined level of perturbation. In certain embodiments, a predefined level of perturbation is a relative heavy chain (HC) peak of about 60% to about 72% and/or a relative light chain (LC) peak of about 30% to about 36%.

In one embodiment, provided is a stable, aqueous formulation comprising:
(a) about 100 mg/ml adalimumab;
(b) about 200 mM to about 275 mM sucrose; and
(c) about 0.1% (w/v) polysorbate 20;
wherein the formulation is essentially free of ionic excipients; and wherein the formulation is about pH 5.0 to about pH 6.0. A pH of about 5.5 is particularly suited to an aqueous formulation herein described. In a related embodiment, the stable aqueous formulation is stable under storage conditions of about 2° C. to about 8° C. for at least 6 months. In a related embodiment, the stable, aqueous formulation is stable under storage conditions of about 25° C. for at least about 14 days. In a related embodiment, the stable, aqueous formulation comprises 250 mM sucrose. In a related embodiment, stability of an aqueous formulation may be determined after storing the formulation at about 2° C. to about 8° C. for at least about 3 months. In a related embodiment, stability of an aqueous formulation may be determined after storing the formulation at about 25° C. for at least about 1 month. In certain embodiments, stability is determined by analysis of a formulation by SEC-HPLC and a predefined level of perturbation. In certain embodiments, a predefined level of perturbation is a relative monomer peak area of adalimumab not less than about 98%. In certain embodiments, stability is determined by analysis of a formulation by CEX-HPLC and a predefined level of perturbation. In certain embodiments, a predefined level of perturbation is a relative acidic species peak area is not more than about 25%. In certain embodiments stability is determined by analysis of a formulation by particle count light obscuration and a predefined level of perturbation. In certain embodiments, a predetermined level of perturbation is not more than about 6000 particles of size equal to or greater than about 10 µm are detected and/or not more than about 600 particles of size equal to or greater than about 25 µm are detected. In certain embodiments, stability is determined by analysis of a formulation by by CE-SDS (non-reducing) and a predefined level of perturbation. In certain embodiments, a predefined level of perturbation is a relative “IgG” peak (intact adalimumab having two heavy chains and two light chains) not less than about 90%. In certain embodiments, stability is determined by analysis of a formulation by CE-SDS (reducing) and a predefined level of perturbation. In certain embodiments, a predefined level of perturbation is a relative heavy chain (HC) peak of about 60% to about 72% and/or a relative light chain (LC) peak of about 30% to about 36%.

### Osmolality of Formulations

Osmolality of a formulation described herein may be between about 240 mOsm/kg and 420 mOsm/kg. In certain embodiments, osmolality of a formulation described herein is between about 300 mOsm/kg and 400 mOsm/kg.

Methods of determining osmolality are not necessarily limited and can comprises any method well known in the art. An exemplary method of measuring osmolality is freezing point depression.

### PH of Formulations

pH of an aqueous formulation described herein may be between about pH 5.0 to about pH 6.0. Particularly suitable pH for an aqueous formulation described herein is a pH of about 5.2 to about 5.8. In certain embodiments, the pH of an aqueous formulation is about pH 5.5.

### Methods for Preparing Stable, Aqueous Formulations

Adalimumab formulations may be prepared by well-known methods of manufacturing an aqueous antibody formulation.

For example, a general method for preparing formulations in small scale for stability studies is as follows: adalimumab is purified and ultrafiled/diafiltered (UF/DF) into water at a concentration of about 130 mg/ml. Stock solution are prepared, such as 2 M Acetate, 1.375 M Trehalose, 1.75 Sucrose, 20% (w/v) Polysorbate 20. Additionally, stock 1 M (or alternatively) 0.5 M NaOH and 1 M (or alternatively 0.5 M) HCl solutions are prepared for pH adjustment. Each stock component of a small-scale formulation is then weighed into a beaker with gentle stirring between components and a pH adjustment as required prior to final dilution to the intended volume.

A general method for preparing an aqueous formulation herein described comprises
(1) exchanging a first solution of an anti-TNFa antibody (preferably adalimumab) into a second solution comprising
   (a) about 200 mM to about 275 mM trehalose or about 200 mM to about 275 mM sucrose; and
   (b) about 25 mM or less acetate buffer or about 25 mM or less succinate buffer,
   wherein the second solution is essentially free of ionic tonicity-adjusting agents; wherein the second solution is essentially free of amino acid stabilizer; wherein the second formulation has a pH of about 5 to about 6; and wherein the concentration of the anti-TNFα antibody (preferably adalimumab) after exchange is about 90 mg/ml to about 125 mg/ml; and
(2) diluting the solution of step (1) with a nonionic surfactant to obtain a concentration of about 0.05% (w/v) to about 0.15% (w/v) nonionic surfactant. In a related method, the concentration of the anti-TNFa antibody (preferably adalimumab prior to step (2) is about 100 mg/ml).

A general method for preparing an aqueous formulation herein described comprises
(1) exchanging a first solution of an anti-TNFa antibody (preferably adalimumab) into a second solution comprising
   (a) about 200 mM to about 275 mM trehalose or about 200 mM to about 275 mM sucrose; and
   (b) about 0.05% (w/v) to about 0.15% (w/v) nonionic surfactant; and wherein the concentration of the anti-TNFa antibody (preferably adalimumab) after exchange is about 90 mg/ml to about 125 mg/ml;
   wherein the second solution is essentially free of ionic excipients; and
(2) diluting the solution of step (1) with a nonionic surfactant to obtain a concentration of about 0.05% (w/v) to about 0.15% (w/v) nonionic surfactant. In a related method, the concentration of the anti-TNFa antibody (preferably adalimumab prior to step (2) is about 100 mg/ml).

### Methods for Determining Stability

It will be understood that formulations stored (or under storage) are comprised in a suitable container prior to storage or prior to exposure to a stress condition. A suitable container includes a container suitable for administration of an aqueous formulation by injection such as a glass vial, pre-filled syringe, and pre-filled injection pen. In an alternative embodiment, an aqueous formulation may be stored in a container suitable for bulk storage, e.g. a container from which final products (e.g a pre-filled injection pen) may be produced, including, for example a single-use bag, a single-use plastic bag. It will be appreciated that a container, e.g. single-use bag, and the like, meets pharmaceutical standards or are otherwise pharmaceutically acceptable or biopharmaceutical grade.

Adalimumab in formulation may degrade, including but not limited to deamidation, oxidation, denaturation, fragmentation, aggregations, and/or deglycosylation. The stability of a formulation may be assessed by one or more techniques well-known in the art. See, for example, Liu et al., BioDrugs (2016) 30:321-338 Magnenat et al., MABS (2017) 9(1):127-139, each of which describes well-known techniques for assessing the degradation of adalimumab. See also the methods described in Example 9.

In one embodiment, a formulation is analysed by one or more of the following techniques, size exclusion High Performance Liquid Chromatography (SE-HPLC) for aggregate analysis; capillary electrophoresis-sodium dodecyl sulfate (CE-SDS) under reducing or non-reducing conditions for analysing degraded protein size variants; Light Obscuration; visual inspection for assessing visible particles; and/or cation exchange-high performance liquid chromatography (CEX-HPLC) for assessing variation in charged species. Such analyses are known in the art. See, e.g. Liu et al., BioDrugs (2016) 30:321-338 Magnenat et al., MABS (2017) 9(1):127-139.

All references cited herein, including patents, patent applications, publications, and databases, are hereby incorporated by reference in their entireties, whether previously stated to be specifically incorporated or not

The invention relates to the following embodiments:
1. A stable, aqueous formulation comprising:
   (a) about 90 mg/ml to about 125 mg/ml anti-TNFa antibody;
   (b) about 200 mM to about 275 mM trehalose or about 200 mM to about 275 mM sucrose;
   (c) about 0.05% (w/v) to about 0.15% (w/v) nonionic surfactant; and
   (d) about 25 mM or less acetate buffer or about 25 mM or less succinate buffer,
   wherein the formulation is essentially free of ionic tonicity-adjusting agents; wherein the formulation is essentially free of amino acid stabilizer; wherein the osmolality of the formulation is about 240 mOsm/kg to about 420 mOsm/kg; and wherein the formulation is about pH 5.0 to about pH 6.0.
2. A stable, aqueous formulation comprising:
   (a) about 90 mg/ml to about 125 mg/ml adalimumab;
   (b) about 200 mM to about 275 mM trehalose or about 200 mM to about 275 mM sucrose;
   (c) about 0.05% (w/v) to about 0.15% (w/v) nonionic surfactant; and
   (d) about 25 mM or less acetate buffer or about 25 mM or less succinate buffer,
   wherein the formulation is essentially free of ionic tonicity adjusting agents; wherein the formulation is essentially free of amino acid stabilizer; wherein the osmolality of the formulation is about 300 mOsm/kg to about 400 mOsm/kg; and wherein the formulation is about pH 5.0 to about pH 6.0.
3. The formulation of embodiment 1 or embodiment 2, comprising about 100 mg/ml adalimumab and/or about 250 mM trehalose.
4. The formulation of embodiment 1 or embodiment 2, comprising about 100 mg/ml adalimumab and/or about 250 mM sucrose.
5. The formulation of any one of embodiments 1 to 4, comprising about 0.1% (w/v) polysorbate 20.
6. The formulation of any one of embodiments 1 to 5, comprising about 20 mM acetate buffer or about 20 mM succinate buffer.
7. The formulation of any one of embodiments 1 to 5, comprising about 20 mM acetate buffer.
8. The formulation of any one of embodiments 1 to 5, comprising about 10 mM acetate buffer.
9. The formulation of any one of embodiments 1 to 5, wherein the formulation is essentially free of ionic excipient.
10. The formulation of embodiment 1 or embodiment 2, comprising about 100 mg/ml adalimumab, about 250 mM trehalose, and about 0.1% polysorbate 20.
11. The formulation of embodiment 1 or embodiment 2, comprising about 100 mg/ml adalimumab, about 250 mM sucrose, and about 0.1% polysorbate 20.
12. The formulation of any one of embodiments 1 to 11, wherein the formulation meets one or more of the following criteria:
   (a) stable under long term storage, wherein long term storage is about 3 months at about 2° C. to about 8° C.;
   (b) stable under long term storage, wherein long term storage is about 6 months at about 2° C. to about 8° C.;
   (c) stable under long term storage, wherein long term storage is about 12 months at about 2° C. to about 8° C.; and/or
   (d) stable under room temperature storage, wherein room temperature storage is about 14 days.
13. The formulation of any one of embodiments 1 to 12, wherein stability is determined by one or more of the following criteria:
   (a) less or about equal increase in aggregation when stored at about 2° C. to about 8° C. for at least about 12 months;
   (b) less or about equal increase in aggregation when stored at about 25° C. for at least 1 month;
   (c) less or about equal increase in relative percent acid species when stored at about 2° C. to about 8° C. for at least 12 months; and/or
   (d) less or about equal increase in relative percent acid species when stored at about 25° C. for at least 1 month;
   wherein the formulation is compared to a control; and wherein said control comprises adalimumab at about the same concentration as the formulation.
14. The formulation of embodiment 13, wherein the formulation comprises about 100 mg/ml adalimumab, and wherein the control consists of about 100 mg/ml adalimumab, 230 mM mannitol, 0.1% (w/v) Polysorbate 80, wherein the control has a pH of about 5.2.
15. The formulation of embodiment 13 or embodiment 14, wherein aggregation is determined by SEC-HPLC and/or wherein relative acid species is determined by CEX-HPLC.
16. The formulation of any one of embodiments 1 to 15, wherein stability is determined by one or more of the following criteria:
   (a) the relative percentage of monomer peak for adalimumab is not less than about 98% after storing the formulation at about 2° C. to about 8° C. for at least 6 months, wherein said relative percentage of monomer peak is determined by SEC-HPLC;
   (b) the relative percentage of monomer peak for adalimumab is not less than about 98% after storing the formulation at about 25° C. for at least 1 month, wherein said relative percentage of monomer peak is determined by SEC-HPLC;
   (c) the relative acidic species peak for adalimumab is not more than about 25% after storing the formulation at about 2° C. to about 8° C. for at least 12 months; wherein said relative acidic species peak is determined by CEX-HPLC; and/or
   (d) the relative acidic species peak for adalimumab is not more than about 25% after storing the formulation at about 25° C. for at least 3 months; wherein said relative acidic species peak is determined by CEX-HPLC.
17. An aqueous formulation comprising
   (a) about 100 mg/ml adalimumab;
   (b) about 200 mM to about 275 mM trehalose;
   (c) about 0.1% (w/v) polysorbate 20; and
   (d) about 20 mM or less acetate buffer;
   wherein the formulation is essentially free of ionic tonicity-adjusting agents; wherein the formulation is essentially free of amino acid stabilizer; and wherein the formulation is about pH 5.0 to about pH 6.0.
18. The formulation of embodiment 17, comprising 250 mM trehalose and 20 mM acetate buffer.
19. An aqueous formulation consisting essentially of
   (a) about 100 mg/ml adalimumab;
   (b) about 200 mM to about 275 mM trehalose;
   (c) about 0.1% (w/v) polysorbate 20; and
   (d) about 20 mM or less acetate buffer;
   wherein the formulation is about pH 5.0 to about pH 6.0.
20. The formulation of embodiment 19, wherein the trehalose concentration is about 250 mM and the acetate buffer concentration is about 20mM.
21. An aqueous formulation comprising
   (a) about 100 mg/ml adalimumab;
   (b) about 200 mM to about 275 mM sucrose;
   (c) about 0.1% (w/v) polysorbate 20; and
   (d) about 20 mM or less acetate buffer;
   wherein the formulation is essentially free of ionic tonicity-adjusting agents; wherein the formulation is essentially free of amino acid stabilizer; and wherein the formulation is about pH 5.0 to about pH 6.0.
22. The formulation of embodiment 21, comprising about 250 mM sucrose and about 20 mM acetate buffer.
23. An aqueous formulation consisting essentially of
   (a) about 100 mg/ml adalimumab;
   (b) about 200 mM to about 275 mM sucrose;
   (c) about 0.1% (w/v) polysorbate 20; and
   (d) about ‘ or less acetate buffer;
   wherein the formulation is about pH 5.0 to about pH 6.0.
24. The formulation of embodiment 23, wherein the sucrose concentration is about 250 mM and the acetate buffer concentration is about 20mM.
25. An aqueous formulation comprising
   (a) about 100 mg/ml adalimumab;
   (b) about 200 mM to about 275 mM trehalose; and
   (c) about 0.1% (w/v) polysorbate 20;
   wherein the formulation is essentially free of ionic excipients; and wherein the formulation is about pH 5.0 to about pH 6.0.
26. The formulation of embodiment 25, comprising 250 mM trehalose.
27. An aqueous formulation consisting essentially of
   (a) about 100 mg/ml adalimumab;
   (b) about 200 mM to about 275 mM trehalose; and
   (c) about 0.1% (w/v) polysorbate 20;
   wherein the formulation is about pH 5.0 to about pH 6.0.
28. The formulation of embodiment 27, wherein the trehalose concentration is about 250 mM,
29. An aqueous formulation comprising
   (a) about 100 mg/ml adalimumab;
   (b) about 200 mM to about 275 mM sucrose; and
   (c) about 0.1% (w/v) polysorbate 20;
   wherein the formulation is essentially free of ionic excipients; and wherein the formulation is about pH 5.0 to about pH 6.0.
30. The formulation of embodiment 29, comprising about 250 mM sucrose.
31. An aqueous formulation consisting essentially of
   (a) about 100 mg/ml adalimumab;
   (b) about 200 mM to about 275 mM sucrose; and
   (c) about 0.1% (w/v) polysorbate 20;
   wherein the formulation is about pH 5.0 to about pH 6.0.
32. The formulation of embodiment 31, wherein the sucrose concentration is about 250 mM,
33. The formulation of any one of embodiments 1 to 11 and 17 to 32, wherein the formulation has been determined to be stable after exposure to one or more of the following stress conditions:
   (a) about 25° C. for about 3 months;
   (b) about 2° C. to about 8° C. for about 3 months;
   (c) about 2° C. to about 8° C. for about 6 months; and/or
   (d) about 40° C. for about 3 months.
34. The formulation of embodiment 33, wherein stability is determined by reference to a predefined level of perturbation.
35. The formulation of embodiment 34, wherein the predefined level of perturbation one or more of
   (a) a relative monomer peak area of adalimumab not less than about 98% as assessed by SEC-HPLC;
   (b) a relative acidic species peak area of adalimumab not more than about 25% as assessed by CEX-HPLC;
   (c) a relative “IgG” peak (intact adalimumab having two heavy chains and two light chains) not less than about 90% as assessed by CE-SDS (non-reducing);
   (d) a relative heavy chain (HC) peak of about 60% to about 72% and/or a relative light chain (LC) peak of about 30% to about 36% as assessed by CD-SDS (reducing); and/or
   (e) not more than about 6000 particles of size equal to or greater than about 10 µm and/or not more than about 600 particles of size equal to or greater than about 25 µm are detected as assessed by particle count light obscuration.
36. The formulation of embodiment 33, wherein stability is determined by reference to a control, wherein said control comprises about 100 mg/ml adalimumab, 230 mM Mannitol, 0.1% (w/v) polysorbate 80, and has a pH of about 5.2; and wherein said control is exposed to the same one or more stress conditions as the formulation.
37. The formulation of embodiment 33, wherein stability is determined with reference to a control wherein said control is a wherein said control is a commercially available Humira^{Ⓡ}; and wherein said control is exposed to the same one or more stress conditions as the formulation.
38. The formulation of any one of embodiments 1 to 37, exhibiting room temperature storage stability.
39. The formulation of any one of embodiments 1 to 37, exhibiting long term storage stability.
40. The formulation of embodiment 39, wherein long term storage stability is at least 3 months at about 2° C. to about 8° C.
41. The formulation of embodiment 39, wherein long term storage stability is at least 6 months at about 2° C. to about 8° C.
42. The formulation of any one of embodiments 1 to 41, wherein formulation is about pH 5.5.
43. A method of formulating a formulation of any one of embodiments 1 to 41, comprising:
   (1) exchanging a first solution of an anti-TNFa antibody (preferably adalimumab)into a second solution comprising:
      (a) about 200 mM to about 275 mM trehalose or about 200 mM to about 275 mM sucrose; and
      (b) about 25 mM or less acetate buffer or about 25 mM or less succinate buffer, wherein the second solution is essentially free of ionic tonicity-adjusting agents;
      wherein the second solution is essentially free of amino acid stabilizer; wherein the second formulation has a pH of about 5.0 to about 6.0, and wherein the concentration of the anti-TNFa antibody (preferably adalimumab) after exchange is about 90 mg/ml to about 125 mg/ml; and
   (2) diluting the solution of step (1) with a nonionic surfactant to obtain a concentration of about 0.05% (w/v) to about 0.15% (w/v) nonionic surfactant.
44. A method of embodiment 43, wherein the second solution comprises:
   about 200 mM to about 275 mM trehalose or about 200 mM to about 275 mM sucrose;
   wherein the second solution is essentially free of ionic excipients.
45. The method of embodiment 43 or embodiment 44, wherein the anti-TNFa antibody is adalimumab.
46. The method of any one of embodiments 43 to 45, wherein the pH of the second solution is about 5.5.
47. The formulation obtained by the method of any one of embodiments 43 to 46.
48. The formulation of any one of embodiments 1 to 42 and 47, wherein the formulation is in the form of a single-dose prefilled injection pen, single-dose prefilled syringe, or single-dose prefilled vial.
49. The formulation of any one of embodiments 1 to 42 and 47, wherein the formulation is in the form of a single-dose prefilled injection pen.
50. The formulation of any one of embodiments 1 to 42 and 47, wherein the formulation is in the form of a single-use bag.
51. An aqueous formulation comprising
   (a) 90 to 110 mg/ml adalimumab;
   (b) about 250 mM trehalose;
   (c) about 0.1% (w/v) polysorbate 20; and
   (d) about 20 mM acetate buffer;
   wherein the formulation is essentially free of ionic tonicity adjusting agents; wherein the formulation is essentially free of amino acid stabilizer; and wherein the formulation is about pH 5.5.
52. An aqueous formulation consisting essentially of
   (a) 90 to 110 mg/ml adalimumab;
   (b) about 250 mM trehalose;
   (c) about 0.1% (w/v) polysorbate 20; and
   (d) about 20 mM acetate buffer;
   wherein the formulation is about pH 5.5.
53. An aqueous formulation comprising
   (a) 90 to 110 mg/ml adalimumab;
   (b) about 250 mM sucrose;
   (c) about 0.1% (w/v) polysorbate 20; and
   (d) about 20 mM acetate buffer;
   wherein the formulation is essentially free of ionic tonicity adjusting agents; wherein the formulation is essentially free of amino acid stabilizer; and wherein the formulation is about pH 5.5.
54. An aqueous formulation consisting essentially of
   (a) 90 to 110 mg/ml adalimumab;
   (b) about 250 mM sucrose;
   (c) about 0.1% (w/v) polysorbate 20; and
   (d) about 20 mM acetate buffer;
   wherein the formulation is about pH 5.5.
55. An aqueous formulation comprising
   (a) 90 to 110 mg/ml adalimumab;
   (b) about 250 mM trehalose; and
   (c) about 0.1% (w/v) polysorbate 20;
   wherein the formulation is essentially free of ionic excipients; and wherein the formulation is about pH 5.5.
56. An aqueous formulation consisting essentially of
   (a) 90 to 110 mg/ml adalimumab;
   (b) about 250 mM trehalose; and
   (c) about 0.1% (w/v) polysorbate 20;
   wherein the formulation is about pH 5.5.
57. An aqueous formulation comprising
   (a) 90 to 110 mg/ml adalimumab;
   (b) about 250 mM sucrose; and
   (c) about 0.1% (w/v) polysorbate 20;
   wherein the formulation is essentially free of ionic excipients; and wherein the formulation is about pH 5.5.
58. An aqueous formulation consisting essentially of
   (a) 90 to 110 mg/ml adalimumab;
   (b) about 250 mM sucrose; and
   (c) about 0.1% (w/v) polysorbate 20;
   wherein the formulation is about pH 5.5
59. The formulation of any one of embodiments 51 to 58, wherein the formulation has been determined to be stable after exposure to one or more of the following stress conditions:
   (a) about 25° C. for about 3 months;
   (b) about 2° C. to about 8° C. for about 3 months;
   (c) about 2° C. to about 8° C. for about 6 months; and/or
   (d) about 40° C. for about 3 months.
60. The formulation of embodiment 59, wherein stability is determined by reference to a predefined level of perturbation.
61. The formulation of embodiment 60, wherein the predefined level of perturbation one or more of
   (a) a relative monomer peak area of adalimumab not less than about 98% as assessed by SEC-HPLC;
   (b) a relative acidic species peak area of adalimumab not more than about 25% as assessed by CEX-HPLC;
   (c) a relative “IgG” peak (intact adalimumab having two heavy chains and two light chains) not less than about 90% as assessed by CE-SDS (non-reducing);
   (d) a relative heavy chain (HC) peak of about 60% to about 72% and/or a relative light chain (LC) peak of about 30% to about 36% as assessed by CD-SDS (reducing); and/or
   (e) not more than about 6000 particles of size equal to or greater than about 10 µm and/or not more than about 600 particles of size equal to or greater than about 25 µm are detected as assessed by particle count light obscuration.
62. The formulation of embodiment 59, wherein stability is determined by reference to a control, wherein said control comprises about 100 mg/ml adalimumab, 230 mM Mannitol, 0.1% (w/v) polysorbate 80, and has a pH of about 5.2; wherein said control is exposed to the same one or more stress conditions as the formulation.
63. The formulation of embodiment 59, wherein stability is determined with reference to a control wherein said control is a wherein said control is a commercially available Humira^{Ⓡ}.
64. The formulation of any one of embodiments 52 to 58, exhibiting room temperature storage stability.
65. The formulation of any one of embodiments 52 to 58, exhibiting long term storage stability.
66. The formulation of embodiment 65, wherein long term storage stability is at least 3 months at about 2° C. to about 8° C.
67. The formulation of embodiment 65, wherein long term storage stability is at least 6 months at about 2° C. to about 8° C.

### EXAMPLES 1 TO 9

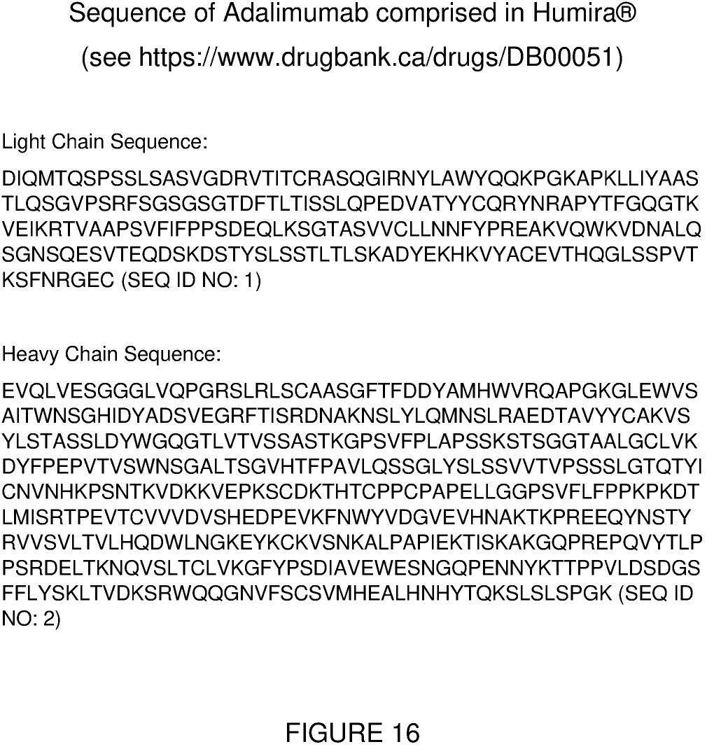

The adalimumab used to in Examples 1 to 9 is an IgG1 antibody with an amino acid sequence corresponding to that comprised in commercial Humira^{Ⓡ}. The adalimumab used in Examples 1 to 9 has been confirmed as comprising a light chain sequence and heavy chain sequence shown in FIG. 16.

### Example 1: Pre-formulation Studies (Inorganic Salts - Ionic Tonicity-Adjusting Agents)

The effects of inorganic salts on thermal stability were tested. Inorganic salts were tested as they could be useful as tonicity adjusting agents.

The effect of inorganic salts on thermal stability of adalimumab was tested Samples of adalimumab were prepared at a final concentration of 0.2 mg/ml, a 1x concentration of SYPRO orange gel stain (Invitrogen) and 5, 25 or 125 mM of five species of salt: magnesium chloride, sodium chloride, sodium metabisulfite, potassium chloride, and calcium chloride. Each sample was distributed into four wells of a 96-well plate (50 µl sample each.) The protein was heated in a 7500 Real Time PCR thermocycler system from Applied Biosystems using a slow gradual heat ramp (machine setting 4%). Fluorescence was recorded over the temperature range 50-95° C. As the protein is heated and denatures, the dye binds to the newly-exposed hydrophobic portions of the protein, and in so doing, increases in fluorescence. This increasing fluorescence generates the melting curve (see e.g. FIG. 1B).

The melting temperature (Tm) of each curve was determined using specialized ProteinThermal Shift™ Software 1x, from Applied Biosystems. Two methods are available for determining the Tm based on the melting curve. The first fits the inflex of the curve to a Boltzmann distribution. The second determines the derivative of the melting curve and determines the peak of the differential curve. In a measurement of adalimumab in water, the two methods yielded highly comparable results. The derivative method is preferable due to its independence of fitting a model.

The derivative method also allows for more than one Tm to be determined for a single measurement. The results of the inorganic salt experiments are shown in FIG. 1A and Table 1.

TABLE 1

| Condition | Tm low (°C) | Tm high (°C) |
|---|---|---|
| No salt (WFI) | N/A | 72.3 + 0.1 |
| 5 mM MgC1₂ | N/A | 71.1 + 0.0 |
| 25 mM MgC1₂ | N/A | 71.8+0.1 |
| 125 mM MgC1₂ | 61.7+ 0.2 | 69.9+ 0.2 |
| 5 mM NaCl | N/A | 71.4+ 0.2 |
| 25 mM NaCl | N/A | 71.6±0.1 |
| 125 mM NaCl | 63.4 + 1.3 | 69.5 + 0.6 |
| 5 mM Na₂S₂O₅ | N/A | 63.4 + 0.9 |
| 25 mM Na₂S₂O₅ | N/A | 54.4 + 0.1 |
| 125 mM Na₂S₂O₅ | N/A | 51.6+ 0.2 |
| 5 mM KCl | N/A | 71.6+ 0.1 |
| 25 mM KCl | N/A | 71.9+ 0.1 |
| 125 mM KCl | 64.4 + 0.2 | 70.1 + 0.2 |
| 5 mM CaC1₂ | N/A | 70.5 + 1.1 |
| 25 mM CaC1₂ | 66.9+ 0.8 | 72.7+ 0.8 |
| 125 mM CaC1₂ | 61.4+ 0.1 | 70.8 + 0.2 |

Table 1 - Determined melting temperatures of adalimumab in the presence of variable concentrations of five inorganic salt species. Each value is an average of four melting temperatures. Errors are standard deviations as determined by STDEV.S in Microsoft Excel.

Table 1 revealed that a second population (or shoulder on the main peak) is present at the higher inorganic salt concentrations (FIGS. 1A to 1F). Two Tm values were given in Table 1 for those conditions for which all the melting curves measured yielded two melting points in the Protein Thermal Shift™ software. In samples that yielded two melting points (Tm high and Tm low), Tm low was typically much lower than Tm high, but Tm high was only modestly lower than the Tm of the WFI condition (e.g. for the 125 mM MgC1₂ condition, Tm high is 69.9° C. compared to the WFI condition Tm, which measures 72.3° C. Tm low, on the other hand, is 61.7° C.)

This result suggests that two distinct populations may be present in the high-salt conditions. The one represented by Tm high may be destabilized by a global effect on the overall protein, thus causing a continuous reduction in Tm with increasing inorganic salt (e.g. reduced strength of hydrogen bonds due to increasing dielectric constant of the environment) whereas the one represented by Tm low has likely undergone some discreet change of state (such as the disruption of a single stabilizing salt bridge). The fact that the two populations appear in four different inorganic salt conditions (MgC1₂, KC1, NaCl, and CaC1₂), suggests that the effect may be a general property (e.g. disruption of the same pre-existing stabilizing bond) rather than as a result of something specific such as binding to one of the inorganic salt ions.

One purpose of including salt in the formulation of parenteral protein drugs is to adjust the tonicity of the injectable solution to be isotonic (c.a. 300 mOsm/kg). The findings indicate that none of the inorganic salts studied is suitable to be the main adjuster of the tonicity of the formulation, as all inorganic salts tested have a destabilizing effect in the current assay at the concentrations typically employed (order of magnitude: 100 mM).

### Example 2: Pre-Formulation Studies (Polyols/Sugar)

Samples of adalimumab were prepared for qPCR measurement in the same way as described for inorganic salts as described in Example 1. Mannitol, sorbitol, sucrose, trehalose, and glycerol were included at concentrations of 5, 25, and 125 mM,

Melting temperatures of these samples are shown in FIG. 2 and listed in Table 2. Some minor variations in Tm were observed. Mannitol, at the highest concentration, represents the lowest measured Tm, at 0.34° C. lower than the control sample without any sugar/polyol. Trehalose showed a trend towards higher Tm at the highest concentrations (0.25° C. greater than for 0 mM trehalose), though the difference is only barely greater than a single standard deviation of the measurements. Overall, the effect of this group of excipients on adalimumab stability is neutral.

TABLE 2

| Condition | Tm (°C) |
|---|---|
| No Sugar/Polyol (WFI) | 72.5 + 0.0 |
| 5 mM Mannitol | 72.1+ 0.1 |
| 25 mM Mannitol | 72.2 + 0.0 |
| 125 mM Mannitol | 72.1+ 0.1 |
| 5 mM Sorbitol | 72.3 + 0.1 |
| 25 mM Sorbitol | 72.4 + 0.1 |
| 125 mM Sorbitol | 72.5 + 0.2 |
| 5 mM Sucrose | 72.4 + 0.1 |
| 25 mM Sucrose | 72.6+ 0.0 |
| 125 mM Sucrose | 72.6+ 0.2 |
| 5 mM Trehalose | 72.5 + 0.1 |
| 25 mM Trehalose | 72.6+ 0.0 |
| 125 mM Trehalose | 72.7+ 0.1 |
| 5 mM Glycerol | 72.5 + 0.1 |
| 25 mM Glycerol | 72.6+ 0.0 |
| 125 mM Glycerol | 72.4 + 0.2 |

Table 2 - Determined melting temperatures of adalimumab in the presence of variable concentrations of five sugar/polyol species. Each value is an average of four melting temperatures. Errors are standard deviations as determined by STDEV.S in Microsoft Excel.

Notably, all melting curves of adalimumab in the presence of the sugar/polyol are single curves and exhibit no shoulder, even at the highest concentration. This further suggests that these excipients are not destablizing to adalimumab.

Isotonicity modification is a key reason for inclusion of sugars/polyols in parenteral protein formulations, although other stabilizing benefits have been observed (e.g. chelating metal ions and therefore serving as antioxidants). As shown in Example 1, inorganic salts (ionic-tonicity adjusting agents) tend to destabilize adalimumab. Due to the slight increase in Tm in the trehalose conditions and the sucrose condition, these sugars were proposed as the primary tonicity modifier for further study.

### Example 3: Pre-Formulation Studies (Buffer and pH)

Five buffers were chosen for screening: citrate, tris, succinate, acetate and phosphate-citrate. The buffering range was evaluated from the theoretical pKa values, and three pH values appropriate for each buffer were chosen from the low, middle and high part of their range. Therefore, a total of fifteen individual buffers were prepared.

Buffers were prepared as follows. Tris (pH 7.1, 8.1, and 9.1), Succinate (pH 3.5, 5, 6.5) and Acetate (4, 4.8, 5.6) buffer samples were each prepared by dissolving tris(hydroxymethyl)aminomethane, succinic acid, and glacial acetic acid to the desired concentration and adjusting pH with HCl or NaOH stock solutions as required. Citrate buffer (pH 3, 5, 7) was made by dissolving citric acid monohydrate and trisodium citrate dihydrate to the desired concentration and mixing to the desired pH (approximate ratio 3:7). Citrate-phosphate buffer was made by dissolving disodium hydrogen phosphate.2H₂0 to the desired concentration, and titrating it against citric acid stock of the same concentration (approximate ration 2.3:1 phosphate:citrate). The final buffer concentrations required were 25 mM for shaking experiments and 10, 25 and 40 mM for thermofluor (melting temperature) experiments.

The stability of adalimumab was found to be highly dependent on pH as clearly demonstrated in FIG. 3 and Table 3. FIG. 3A shows that the melting temperature remains constant in every condition in the pH range of approximately 4 or 5 to 9.

The pH dependence of adalimumab is borne out just as clearly when examining the populations evident in the melting curves in FIGS. 3B to 3F. For each curve measured at high pH (above pH 5 or 6), a single population is evident, essentially overlapping that of water (e.g. the Tris series melting curves.) For others, like citrate at pH 4.26, a second population becomes evident, and at the lowest pH, such as citrate at pH 2.81, a single low-temperature melting curve is evident without a population at the higher temperature.

Interestingly, unlike the inorganic salt series (Example 1), the effect of destabilizing pH appears not to decrease Tm high and cause a second population to appear in the same measurement. Rather, the effect appears to be bimodal, where the Tm is either the same as in the water conditions, or Tm low (e.g. in the phosphate-citrate series, FIG. 3.)

The implication of this finding may be that the destabilization adalimumab due to pH may occur through a discreet state as discussed in the section on inorganic salts (Example 1) and not a global destabilization. For example, a lower pH could protonate an amino acid critical for a salt-bridge, thus breaking the salt-bridge and destabilizing the protein (conceivably even at the same site as salt, even though the mechanism of destabilization may be different).

TABLE 3

| Buffer Condition | pH | Tm (°C) |
|---|---|---|
| Citrate (low) | 2.81 | 56.4+ 0.1 |
| Phosphate-citrate (low) | 3.23 | 59.3 + 0.0 |
| Succinate (low) | 3.53 | 64.4+ 0.1 |
| Acetate (low) | 4.1 | 70.4+ 0.2 |
| Citrate (mid) | 4.26 | 68.4+ 0.0 |
| Acetate (mid) | 4.81 | 71.8±0.0 |
| Succinate (mid) | 5.03 | 71.1 + 0.0 |
| Acetate (high) | 5.69 | 71.8+0.3 |
| Citrate (high) | 5.69 | 70.6+ 0.2 |
| Phosphate -citrate (mid) | 5.74 | 71.8+0.0 |
| Succinate (high) | 6.53 | 71.5 + 0.2 |
| No buffer) | N/A | 71.3 + 0.4 |
| Tris (low) | 7.52 | 71.6+0.1 |
| Phosphate-citrate (high) | 7.64 | 71.3 + 0.3 |
| Tris (mid) | 8.27 | 71.9±0.0 |
| Tris (high) | 9.02 | 71.5 + 0.2 |

Table 3 - Determined melting temperatures of adalimumab at various pH values. Each value is an average of four melting temperatures. Errors are standard deviations as determined by STDEV.S in Microsoft Excel.

### Example 4: Pre-Formulation Studies (Agitative Stability and pH)

Because pH is widely considered and as shown in Example 3 to be a particularly important contributor to protein stability, variable pH conditions were also tested against an agitative (shaking) stressor. Briefly, 1 M buffer stock and adalimumab were mixed to a concentration of 99 mg/ml adalimumab and 25 mM buffers at variable pH values. Two milliliters of each solution were placed in 6 R vials and sealed with a stopper and overseal. The vials were then shaken for four days at 300 rpm or stored in fridge without shaking as a negative control. Each experiment also included samples of the protein in water (no buffer), both shaken and not shaken, as an additional control. Because the protein concentration was limited to 108 mg/ml, a 1 M stock of buffer was required to reach the desired buffer concentration. Due to solubility limitations of the succinate and phosphate-citrate buffers, it was not possible to include these in the shaking assay. Nonetheless, the full desired pH range of 3-9 was represented in experiment. The samples were then analyzed by turbidity measurements. (Spectrophotometer absorbance measurement at 320 nm in 1 ml pathlength cuvette.)

The findings of the shaking assay are represented in FIG. 4. The error bars are the standard deviations of three readings and are much larger than in the Thermofluor assay. An overall trend is apparent, however, and appears to be in opposition to the findings of the Thermofluor assay (Example 3). Namely, the lower the pH, the more resilient adalimumab appears to be against shaking stress. Therefore, while the Thermofluor results (Example 3) indicate that any pH above 5 is a stabilizing condition, these results suggest that the protein should be formulated at the lower end of the thermostable pH range. A nominal pH of about 5.5 is proposed. This allows for optimum stability of adalimumab, as concluded from Example 3 and this example, but allows tolerances in production (e.g. +0.5 pH) that are still within the stable pH range.

### Example 5: Pre-Formulation Studies (Buffer)

While Example 4 indicates an optimum pH range for adalimumab, there are multiple ways to accomplish buffering around pH 5. Four of the tested buffers are effective around pH 5, in addition to which the adalimumab itself is self-buffering and does not strictly require a buffering system at higher concentrations (e.g. about 100 mg/ml and above) (see Example 7). See, e.g., Gokam et al., J Pharm Sci (2008) 97(8): 3051-3066. The current experiment, therefore, was meant to discover if any of the suitable buffers would confer stabilizing effects that would make it a better choice over the others or over a buffer-free composition.

In this experiment, succinate, citrate, phosphate-citrate, and acetate buffers, at various concentrations, were adjusted to the same pH of 5.2 and assessed by the Thermofluor assay. Concentrations of the buffers were 10, 25 and 40 mM. The final concentration of adalimumab was 0.2 mg/ml, and SYPRO orange was 1x, as in Example 1.

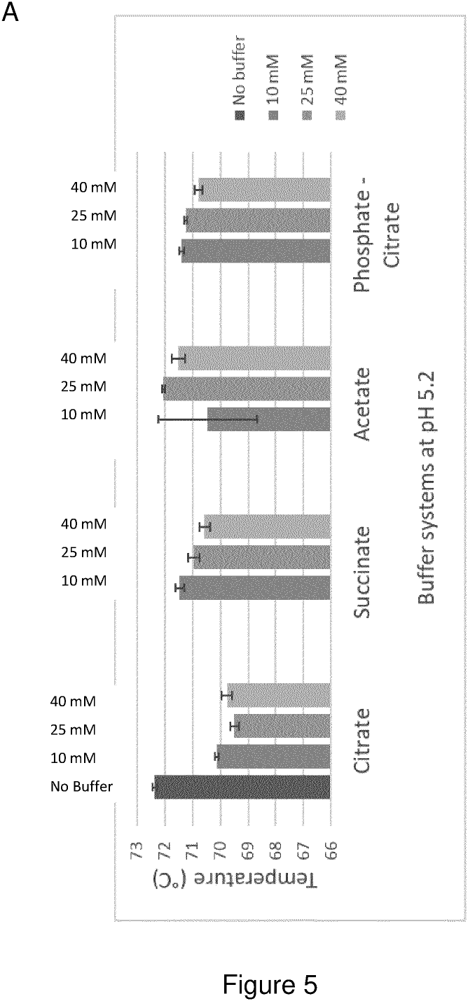

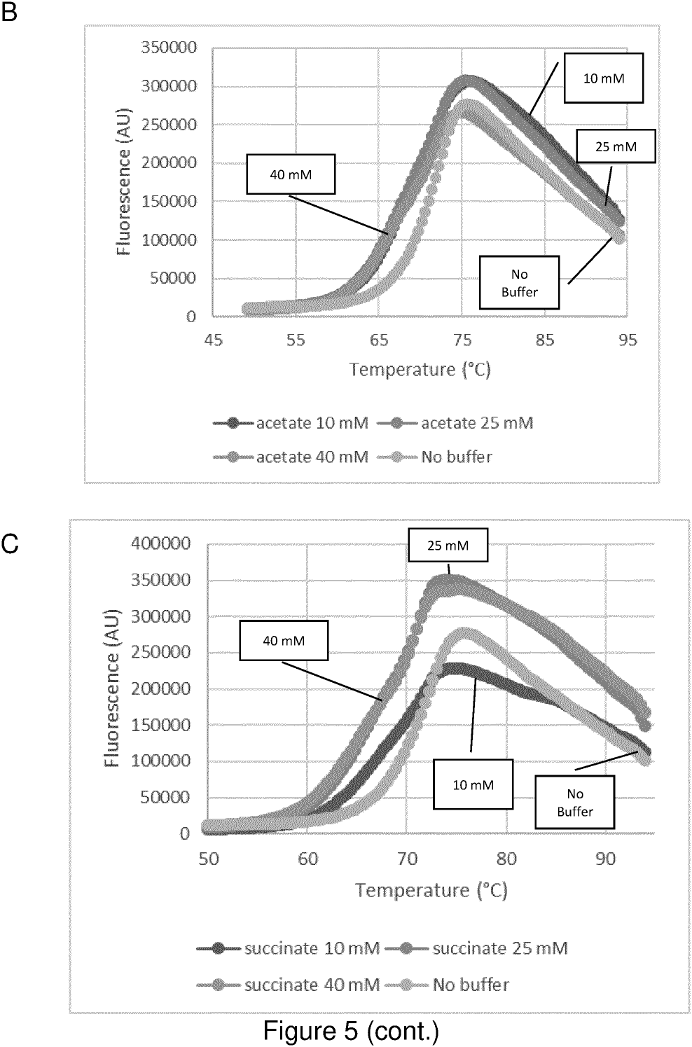

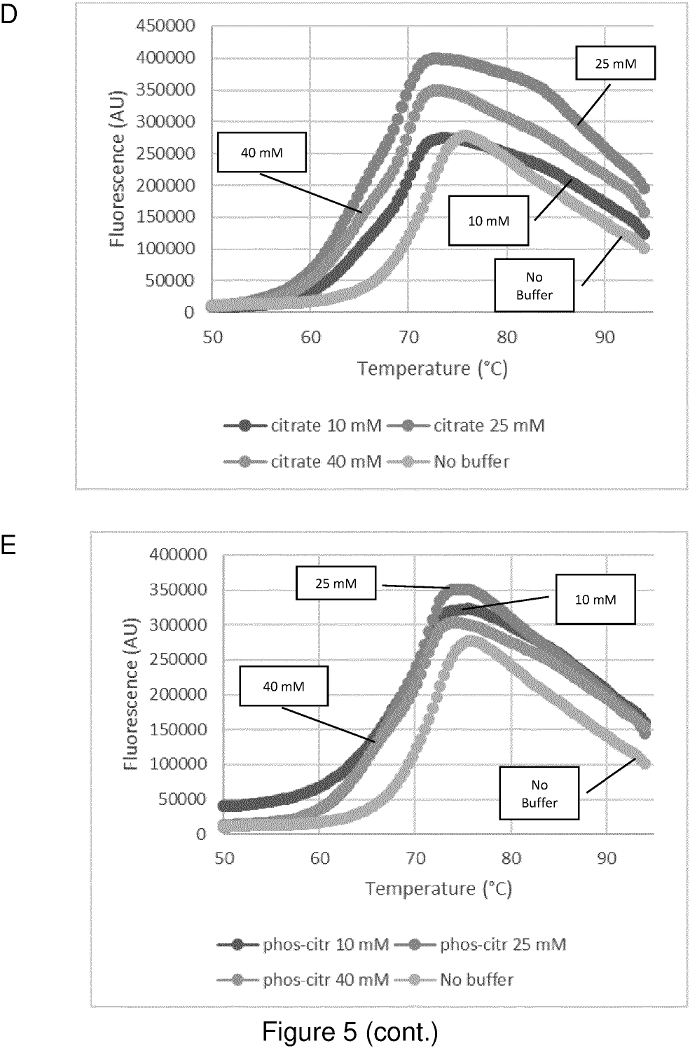

The results indicate that none of the tested buffers had a stabilizing or measurably positive effect on the Tm of adalimumab (Table 4, FIG. 5). Rather, all buffer systems tended towards a concentration-dependent decrease in Tm. Citrate appeared to be the most destabilizing and acetate the least.

TABLE 4

| Buffer condition | pH | Tm (°C) |
|---|---|---|
| No-buffer (WFI) | 5.2 | 72.4 + 0.1 |
| 10 mM citrate | 5.2 | 70.1±0.1 |
| 25 mM citrate | 5.2 | 69.5 +0.1 |
| 40 mM citrate | 5.2 | 69.8+ 0.2 |
| 10 mM succinate | 5.2 | 71.5 ±0.1 |
| 25 mM succinate | 5.2 | 70.9± 0.2 |
| 40 mM succinate | 5.2 | 70.6± 0.2 |
| 10 mM acetate | 5.2 | 70.5 + 1.8 |
| 25 mM acetate | 5.2 | 72.0± 0.1 |
| 40 mM acetate | 5.2 | 71.5 ±0.2 |
| 10 mM phosphate citrate | 5.2 | 71.4± 0.1 |
| 25 mM phosphate citrate | 5.2 | 71.2±0.1 |
| 40 mM phosphate citrate | 5.2 | 70.8 ± 0.3 |

Table 4: Determined melting temperatures of adalimumab at various concentrations of buffers. Each value is an average of four melting temperatures. Errors are standard deviations as determined by STDEV.S in Microsoft Excel.

### Example 6: Pre-Formulation Studies (Non-Ionic Surfactant)

The primary role of a nonionic surfactant (e.g. polysorbate) in a protein parenteral formulation is to protect against agitative stress. In the current experiment, the shaking assay was performed in the presence of polysorbate 20 and polysorbate 80. Polysorbate is not compatible with the Thermofluor assay, so complementary thermal stability data could not be collected.

Two milliliter samples consisting of 99 mg/ml adalimumab and 0.1% (w/v) polysorbate 20 or 80 were placed in 6 R vials, sealed, and agitated at 300 rpm at ambient temperature for 4 days before the samples were analyzed for turbidity. Control samples without any polysorbate were also tested.

The results (shown in FIG. 6) indicate that either polysorbate is highly effective at protecting against agitative stress. In the presence of either polysorbate, the level of turbidity measured is almost the same as that of the control samples which had not been subjected to any shaking in the study. However, comparing polysorbate 20 and 80, polysorbate 20 appears to be measurably more effective at protecting the protein (beyond the error bars, which are standard deviations of three samples.)

### Example 7: Pre-Formulation Studies (Amino Acid Stabilizer)

L-arginine has been increasingly introduced into protein formulations recently due to its ability to stabilize against aggregation. It has been observed to stabilize against agitative rather than thermal stress but in the current experiment, both Thermofluor and shaking assays were performed. The shaking experiment was performed as in Example 4, in the presence of 200 mM L-Arginine. The Thermofluor assay was performed as in Example 1 in the presence of both 50 and 200 mM L-Arginine to gauge any concentration-dependent effects. As shown in Table 5, from the decrease in Tm, addition of L-Arginine tended to destabilize adalimumab. See also FIG. 7.

TABLE 5

| Condition | Tm (°C) |
|---|---|
| No stabilizer | 71.3 ±0.4 |
| L-arginine 50 mM | 70.4 ± 0.1 |
| L-arginine 200 mM | 68.9± 0.1 |

Table 5 - Determined melting temperatures of adalimumab at variable concentrations of amino acid stabilizer L-Arginine. Each value is an average of four melting temperatures. Errors are standard deviations as determined by STDEV.S in Microsoft Excel.

### Example 8: Self-Buffering by Adalimumab

The following experiment was conducted to gauge the extent of self-buffering behavior of adalimumab, and how it changes over the pH range.

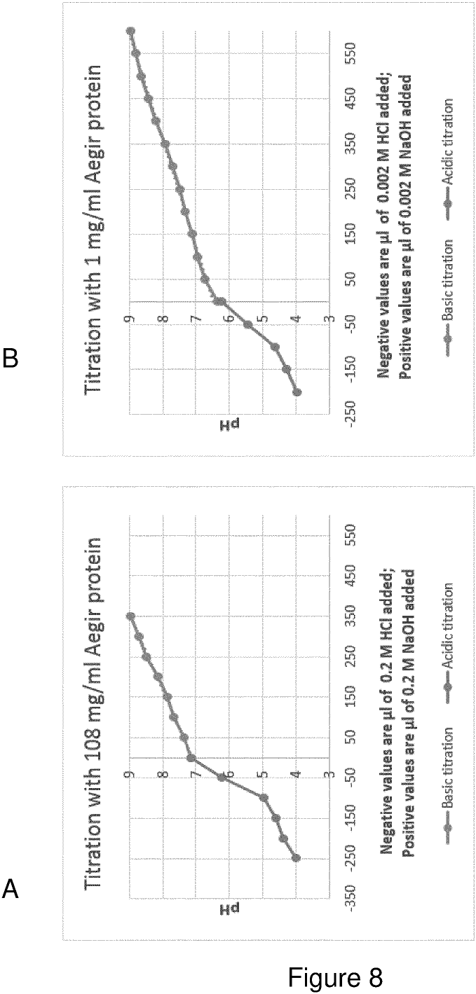

The experiment was conducted by portioning 5 ml of protein (108 mg/ml) into a container and titrating 50 µl of 0.2 M NaOH at a time while stirring, until a pH of about 9 was reached. The acidic titration was conducted in the same fashion, starting with a fresh 5 ml aliquot of protein and adding 50 µl of 0.2 M HCl at a time until a pH of about 4 was reached (FIG. 8A).

The results (shown in FIG. 8) indicate that adalimumab has substantial buffering capacity over a wide range of pH. The relationship is effectively linear, except over the range pH 5-7. Furthermore, the curve in FIG. 8A gives an indication of the amount of acid or base required to adjust the pH if necessary during manufacturing of a formulation. For example, the slope of the basic curve is 0.0053 pH units per µl, for a starting volume of 5 ml, and can be used to estimate acid or base volumes required for pH adjustments of larger volumes of adalimumab. This approach may not be fully predictive for a final formulation, however, as other excipients (e.g. sucrose and glycerol) can affect buffer capacities of some buffer systems.

A second set of titrations was conducted with 1 mg/ml adalimumab and 0.002 M HCl and NaOH. (FIG. 8B). It is striking that this titration is highly similar to the one conducted with 108 mg/ml adalimumab. This suggests that the relative buffer capacity is comparable at different concentrations of mAb protein, even though self-buffering formulations may not be pragmatic for offsetting any pH fluctuations during, for example, formulation, until higher concentrations of about 100 mg/ml are reached.

### Conclusions: Pre-formulation Studies

The overall goals of this study were to determine the stabilizing or destabilizing effect of a range of individual excipients, determine the pH of maximum stability for the protein and to hypothesize stabilizing formulations.

A range of salts, sugars/polyol, pH values, buffering systems, and a stabilizer were tested using a shaking assay and/or Thermofluor assay as appropriate. Adalimumab was categorically destabilized by the presence of ionic excipients such as inorganic salt and buffer in a concentration-dependent manner, though low concentrations of each were well tolerated. Furthermore, adalimumab was thermo-destabilized by pH values lower than 5, but was very thermo-stable at all tested pH values above 5. The shaking stability trend, however, suggested agitative stability increased with decreasing pH. The effect of sugars on adalimumab was essentially neutral. Trehalose was deemed to be the most favorable sugar for tonicity modification. Both polysorbates tested were very effective at stabilizing adalimumab against agitative stress, though polysorbate 20 measurably more so. Finally, while L-arginine increased agitative stability of adalimumab, the increase was modest and the thermo-stability decreased, eliminating this excipient from consideration.

The top formulation candidate suggested on the pre-formulation studies of Examples 1 to 7, therefore, is adalimumab at about 100 mg/ml, with about 0.1% (w/v) polysorbate 20, with trehalose for isotonicity adjustment, and self-buffered at about pH 5.5. An alternative formulation candidate comprises adalimumab at about 100 mg/ml, with about 0.1% (w/v) polysorbate 20, with sucrose for isotonicity adjustment, and self-buffered at about pH 5.5.

Because low concentrations of buffer, particular citrate and succinate, were tolerated, the manufacturing process may include a low concentration of a buffer (rather than self-buffering). Thus, the second formulation to test includes an acetate buffer, the best-tolerated buffering system, at a low concentration of 20 mM. Finally, the same formulations with sucrose in place of trehalose are also proposed for further study.

### Example 9: Formulations of Adalimumab (Stability Analysis)

Four formulations were prepared for stability analysis (as shown in Table 6):

TABLE 6

| Formulation | Buffer | pH | Sugar | Surfacant |
|---|---|---|---|---|
| A | 20 mM Acetate | 5.5 | 250 mM Trehalose | 0.1% (w/v) polysorbate 20 (PS20) |
| B | NA | 5.5 | 250 mM Trehalose | 0.1% (w/v) PS 20 |
| C | 20 mM Acetate | 5.5 | 250 mM Sucrose | 0.1% (w/v) PS 20 |
| D | NA | 5.5 | 250 mM Sucrose | 0.1% (w/v) PS 20 |

The formulations were prepared on the basis of the following general method: is purified and ultrafiltered/diafiltered (UF/DF) into water to a concentration of about 130 mg/ml. Stock solutions are prepared, such as 2 M Acetate, 1.375 M Trehalose, 1.75 Sucrose, 20% (w/v) Polysorbate 20. Additionally, stock 1 M NaCl and 1 M HCl solutions are prepared for pH adjustment. Each stock component is then weighed into a beaker (the order of addition may be protein, sugar, acetate, polysorbate, pH adjustment, water) with gentle stirring between components and a pH adjustment as required prior to final dilution to the intended volume.

A control was used for all studies. The control adalimumab were prepared on the basis of the following general method. Adalimumb is purified and ultrafiltered/diafiltered (UF/DF) into a 230 mM mannitol solution and concentration of 127.5 mg/ml. Stock solutions of 20% (w/v) Polysorbate 80 and 460 mM mannitol are prepared. Additionally, stock 1 M NaCl and 1 M HCl solutions are prepared for pH adjustment. Each stock component was then weighed into a beaker (the order of addition may be protein, sugar, polysorbate, pH adjustment, water) with gentle stirring between components and a pH adjustment as required prior to final dilution to the intended volume. The final control has a concentration of 100 mg/ml adalimumab, 230 mM mannitol, 0.1% (w/v) Polysorbate 80, at a pH of 5.2.

Samples of each formulation are filled (1ml fill volume) into a 1ml long staked-needle prefillable syringe and stoppered with a plunger stopper. 132 samples of each formulation in Table 6 as well as the control were placed on stability.

Samples were stored at 2° C. to 8° C., 25° C., and 40° C. The stability of was determined at time points after storage 0, 1 month, 3 months, 6 months and 12 months. 12 months determination was determined only for storing at 2° C. to 8° C.

### Aggregate Determination of Formulations A to D

Aggregate was determined by size exclusion-high performance liquid chromatography (SEC-HPLC).

The size exclusion method is performed using an Agilent Infinity 1260 instrument equipped with a binary pump, autosampler and a diode array detector. The separation is achieved on a TSKGel SuperSW mAb HR, 4 µm300 mm x 7.8 mm, 250A column, from Tosho Bioscience, under isocratic conditions using a 0.1 M sodium phosphate / 0.1 M sodium chloride, pH 6.2 mobile phase. Test samples are prepared by diluting the mAb solution to 5 mg/mL with mobile phase. Instrument control, data acquisition and reporting is performed using Empower 3 software (Waters).

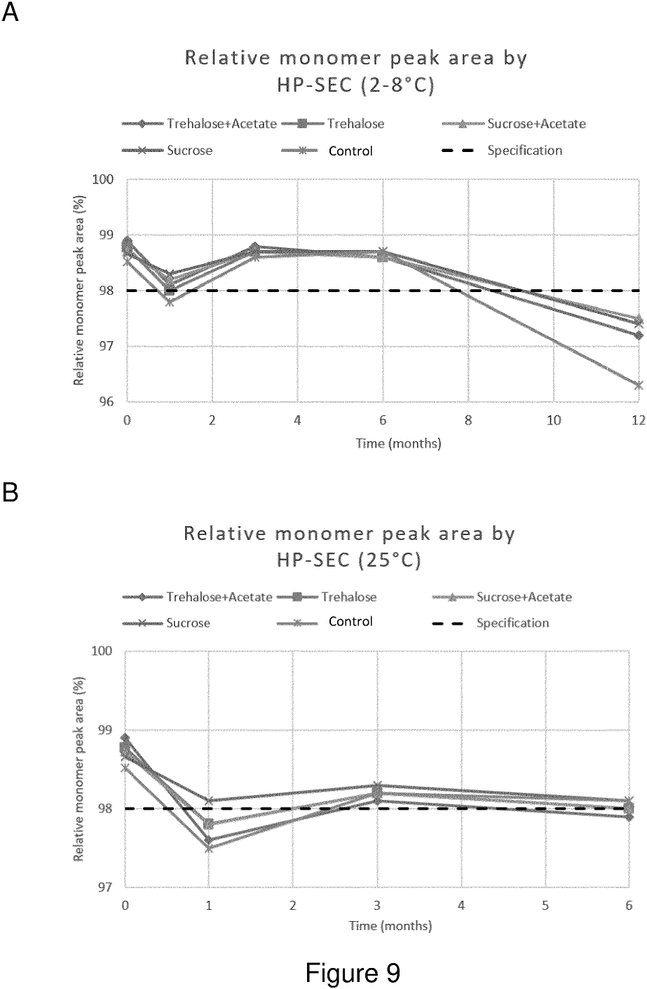

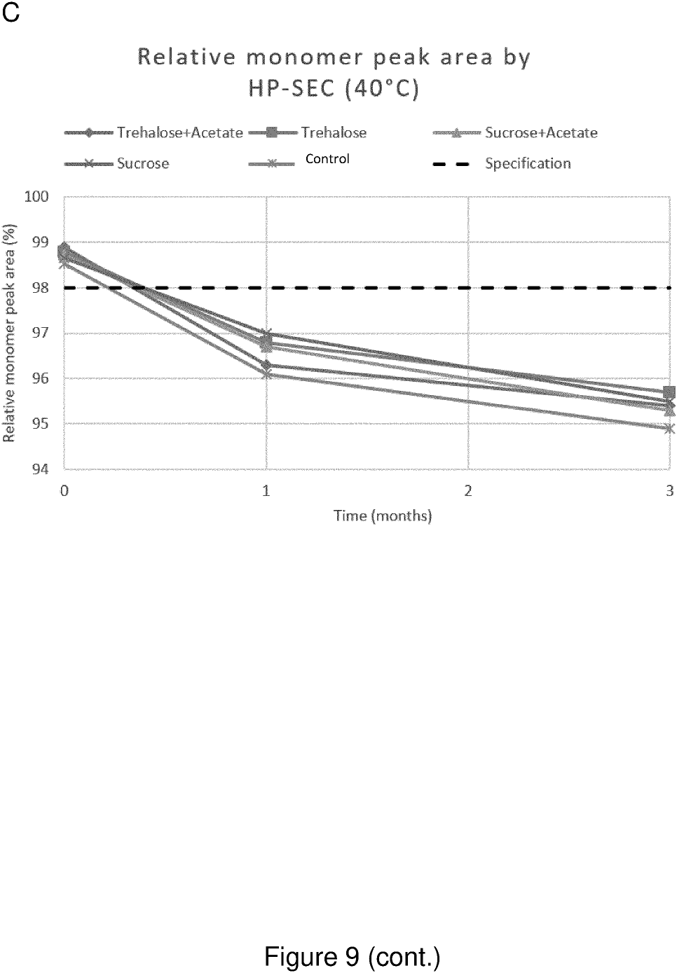

The results of the analyses are shown in Table 7. FIGS. 9A to 9C shows the relative monomer peak area for each storage condition reported in Table 7.

TABLE 7

| | | 2-8° C. | | | 25° C. | | | 40° C. | | |
|---|---|---|---|---|---|---|---|---|---|---|
| Formulation | Time (m) | Aggregation (%) | Main (%) | Degradation (%) | Aggregation (%) | Main (%) | Degradation (%) | Aggregation (%) | Main (%) | Degradation (%) |
| A | 0 | 1.1 | 98.9 | 0 | 1.1 | 98.9 | 0 | 1.1 | 98.9 | 0 |
| | 1 | 1.9 | 98.1 | 0 | 2.3 | 97.6 | 0.1 | 2.8 | 96.3 | 0.9 |
| | 3 | 1.2 | 98.8 | 0 | 1.7 | 98.1 | 0.2 | 2.4 | 95.4 | 2.2 |
| | 6 | 1.4 | 98.6 | 0 | 2 | 97.9 | 0.1 | NA | NA | NA |
| | 12 | 2.7 | 97.2 | 0.1 | NA | NA | NA | NA | NA | NA |
| | | 2-8° C. | | | 25° C. | | | 40° C. | | |
| Formulation | Time (m) | Aggregation (%) | Main (%) | Degradation (%) | Aggregation (%) | Main (%) | Degradation (%) | Aggregation (%) | Main (%) | Degradation (%) |
| B | 0 | 1.22 | 98.78 | 0 | 1.22 | 98.78 | 0 | 1.22 | 98.78 | 0 |
| | 1 | 2 | 98 | 0 | 2.2 | 97.81 | 0 | 2.3 | 96.8 | 0.9 |
| | 3 | 1.3 | 98.7 | 0 | 1.6 | 98.2 | 0.2 | 2.1 | 95.7 | 2.2 |
| | 6 | 1.4 | 98.6 | 0 | 1.9 | 98 | NA | NA | NA | NA |
| | 12 | 2.6 | 97.4 | 0.1 | NA | NA | NA | NA | NA | NA |
| | | 2-8° C. | | | 25° C. | | | 40° C. | | |
| Formulation | Time (m) | Aggregation (%) | Main (%) | Degradation (%) | Aggregation (%) | Main (%) | Degradation (%) | Aggregation (%) | Main (%) | Degradation (%) |
| C | 0 | 1.28 | 98.72 | 0 | 1.28 | 98.72 | 0 | 1.28 | 98.72 | 0 |
| | 1 | 1.8 | 98.2 | 0 | 2.1 | 97.8 | 0.1 | 2.4 | 96.7 | 0.9 |
| | 3 | 1.3 | 98.7 | 0 | 1.7 | 98.2 | 0.2 | 2.4 | 95.3 | 2.2 |
| | 6 | 1.4 | 98.6 | 0 | 1.9 | 98.0 | 0.1 | NA | NA | NA |
| | 12 | 2.5 | 97.5 | 0.1 | NA | NA | NA | NA | NA | NA |
| | | 2-8° C. | | | 25° C. | | | 40° C. | | |
| Formulation | Time (m) | Aggregation (%) | Main (%) | Degradation (%) | Aggregation (%) | Main (%) | Degradation (%) | Aggregation (%) | Main (%) | Degradation (%) |
| D | 0 | 1.34 | 98.66 | 0 | 1.34 | 98.66 | 0 | 1.34 | 98.66 | 0 |
| | 1 | 1.7 | 98.3 | 0 | 1.9 | 98.1 | 0 | 2.1 | 97 | 0.9 |
| | 3 | 1.3 | 98.7 | 0 | 1.6 | 98.3 | 0.2 | 2.2 | 95.5 | 2.3 |
| | 6 | 1.3 | 98.7 | 0 | 1.8 | 98.1 | 0.1 | NA | NA | NA |
| | 12 | 2.5 | 97.4 | 0.1 | NA | NA | NA | NA | NA | NA |
| | | 2-8° C. | | | 25° C. | | | 40° C. | | |
| Formulation | Time (m) | Aggregation (%) | Main (%) | Degradation (%) | Aggregation (%) | Main (%) | Degradation (%) | Aggregation (%) | Main (%) | Degradation (%) |
| Control | 0 | 1.48 | 98.52 | 0 | 1.48 | 98.52 | 0 | 1.48 | 98.52 | 0 |
| | 1 | 2.2 | 97.8 | 0 | 2.4 | 97.5 | 0.1 | 2.7 | 96.1 | 1.2 |
| | 3 | 1.4 | 98.6 | 0 | 1.6 | 98.2 | 0.2 | 2.1 | 94.9 | 2.9 |
| | 6 | 1.3 | 98.7 | 0 | 1.7 | 98.1 | 0.2 | NA | NA | NA |
| | 12 | 3.6 | 96.3 | 0.1 | NA | NA | NA | NA | NA | NA |

### Subvisible Particle Concentration Determination of Formulations A to D

Subvisible particle concentration was determined micro-flow imagining (MFI).

The sub-visible particle measurement using micro flow imaging is performed using a MFI 5200 instrument equipped with Bot1 autosampler (Protein Simple). Samples are measured undiluted (4 x 0.9 mL) and special care is taken not to introduce air bubbles or particle contaminants by transferring samples into sample vials under LAF conditions. Instrument control, data acquisition and reporting is performed using the Protein Simple MFI software MVSS and MVAS.

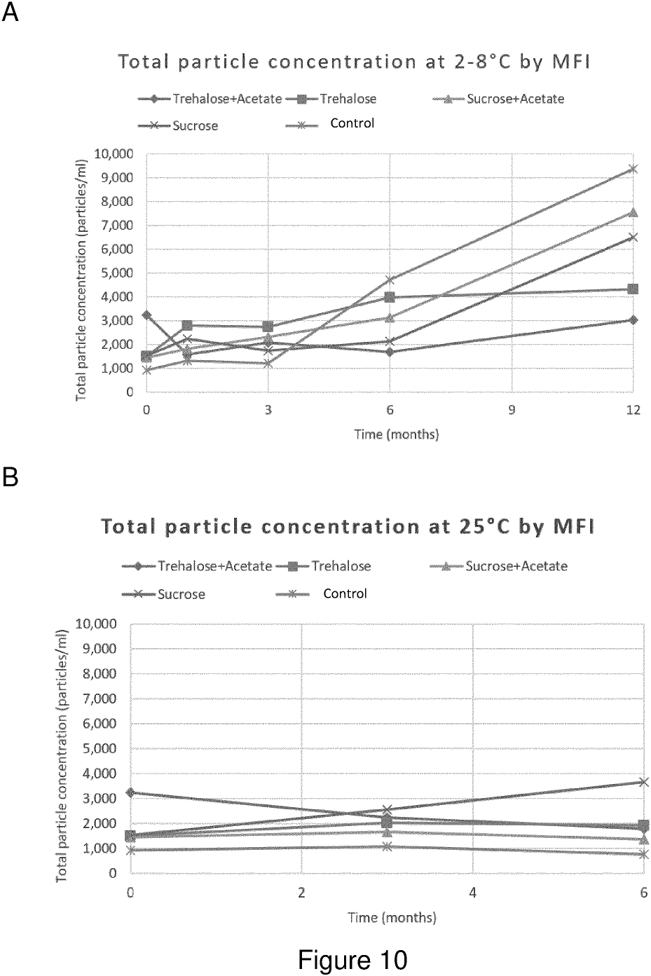

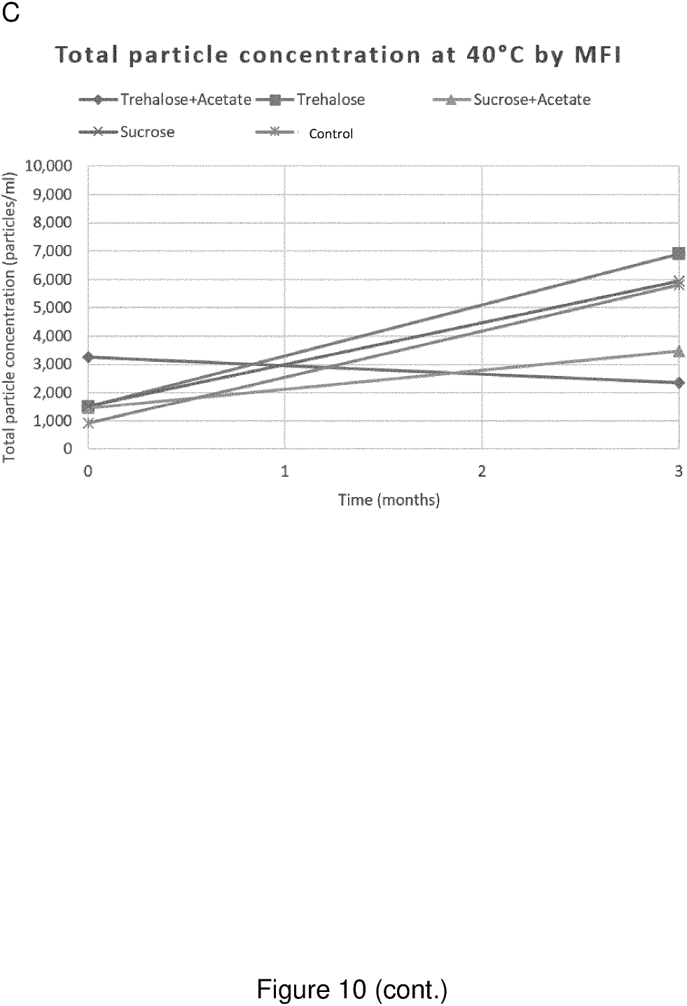

The results of the analyses are shown in Table 8. FIGS. 10A to 10C show the total particle concentration for each storage condition.

TABLE 8

| | | Total Particle Concentration (particles/mL) | | |
|---|---|---|---|---|
| Formulation | Time (m) | 2-8° C. | 25° C. | 40° C. |
| A | 0 | 3252 | 3252 | 3252 |
| | 1 | 1570 | N.A. | N.A. |
| | 3 | 2086 | 2235 | 2344 |
| | 6 | 1678 | 1798 | N.A. |
| | 12 | 3018 | N.A. | N.A. |

| | | Total Particle Concentration (particles/mL) | | |
|---|---|---|---|---|
| Formulation | Time (m) | 2-8° C. | 25° C. | 40° C. |
| B | 0 | 1493 | 1493 | 1493 |
| | 1 | 2801 | N.A. | N.A. |
| | 3 | 2731 | 2022 | 6902 |
| | 6 | 3975 | 1921 | N.A. |
| | 12 | 4310 | N.A. | N.A. |
| | | Total Particle Concentration (particles/mL) | | |
| Formulation | Time (m) | 2-8° C. | 25° C. | 40° C. |
| C | 0 | 1448 | 1448 | 1448 |
| | 1 | 1808 | N.A. | N.A. |
| | 3 | 2305 | 1655 | 3464 |
| | 6 | 3138 | 1368 | N.A. |
| | 12 | 7570 | N.A. | N.A. |
| | | Total Particle Concentration (particles/mL) | | |
| Formulation | Time (m) | 2-8° C. | 25° C. | 40° C. |
| D | 0 | 1521 | 1521 | 1521 |
| | 1 | 2228 | N.A. | N.A. |
| | 3 | 1739 | 2556 | 5947 |
| | 6 | 2138 | 3657 | N.A. |
| | 12 | 6493 | N.A. | N.A. |
| | | Total Particle Concentration (particles/mL) | | |
| Formulation | Time (m) | 2-8° C. | 25° C. | 40° C. |
| Control | 0 | 915 | 915 | 915 |
| | 1 | 1307 | N.A. | N.A. |
| | 3 | 1204 | 1072 | 5794 |
| | 6 | 4714 | 771 | N.A. |
| | 12 | 9375 | N.A. | N.A. |

### Charged Species Determination of Formulations A to D

Relative main peak, acid species peak, and basic peak determined by cation exchange-high performance liquid chromatography (CEX-HPLC).

The cationic exchange chromatography (CEX) is performed using an Agilent Infinity II 1260 bioinert instrument equipped with a quaternary pump, autosampler and a diode array detector. The separation is achieved on a MabPac SCX-10, 10 µm, 250 mm x 4.0 mm column, from Thermo Scientific, using a salt gradient with mobile phase A as 10 mM sodium phosphate, pH 7.0 and mobile phase B as 10 mM sodium phosphate / 100 mM sodium chloride, pH 7.0. Test samples are prepared by diluting the mAb solution to 0.5 mg/mL with mobile phase A with 0.1% polysorbate 80. Instrument control, data acquisition and reporting is performed using Empower 3 software (Waters).

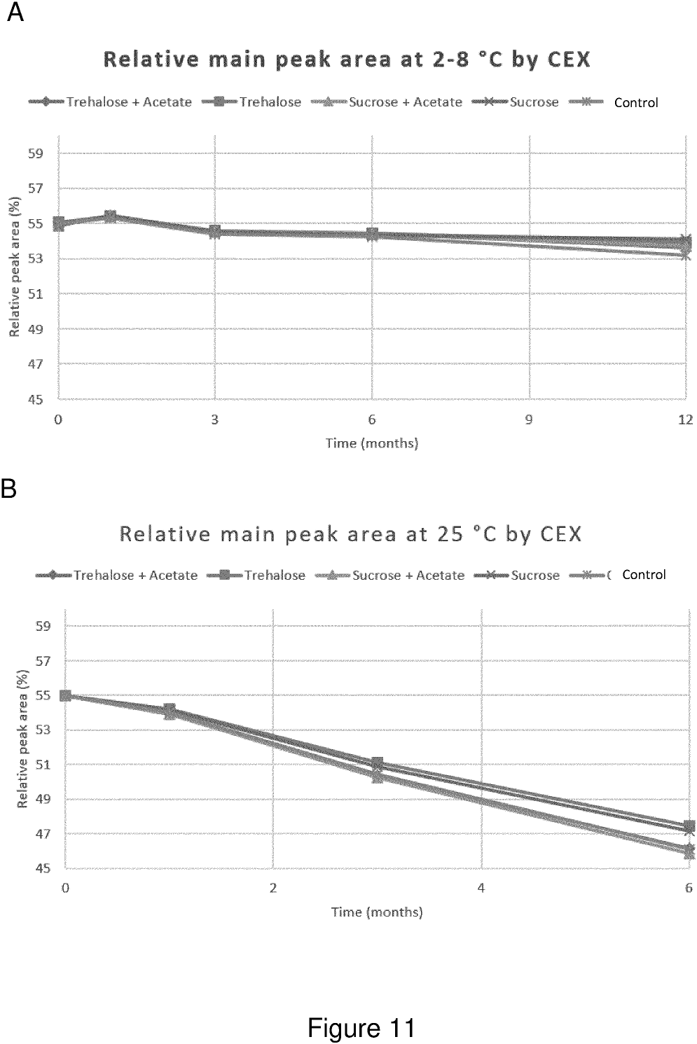

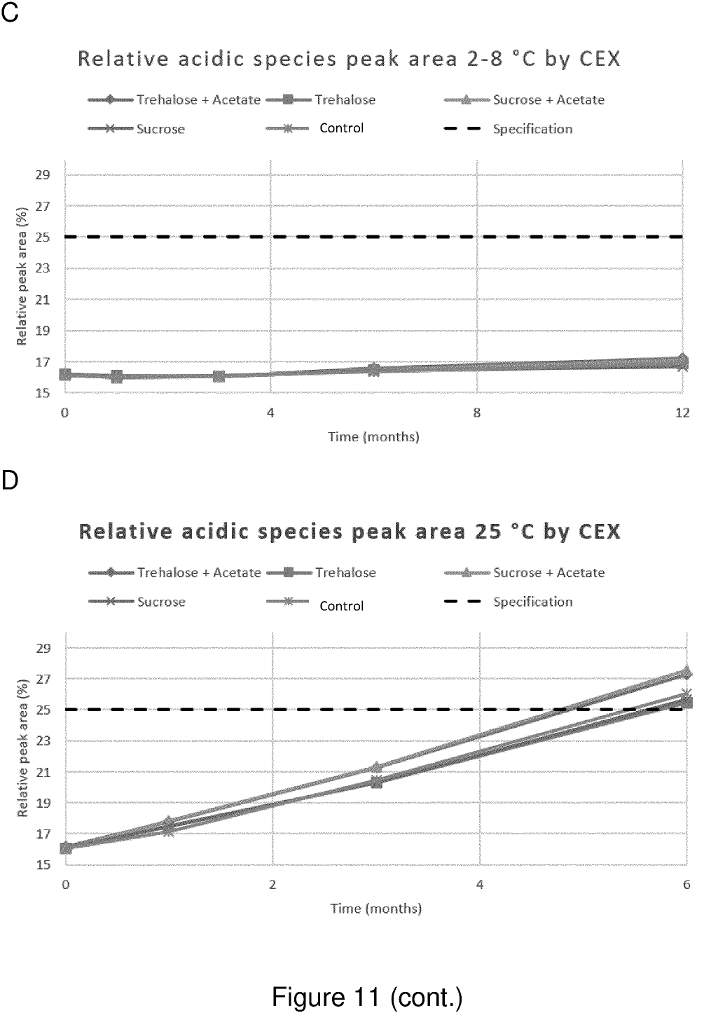

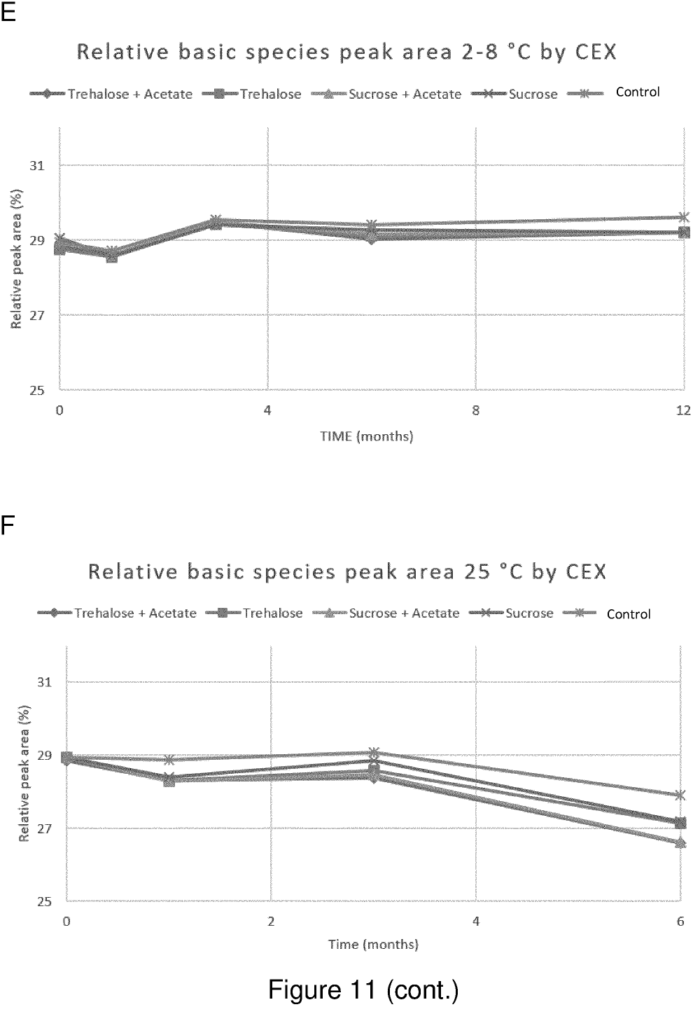

The results of the analyses are shown in Table 9. FIGS. 11A to 11F show the relative main peak, relative acidic species, and relative basic species for 2° C. to 8° C. and 25° C. storage condition.

TABLE 9

| | | 2-8° C. | | | 25° C. | | |
|---|---|---|---|---|---|---|---|
| Formulation | Time (m) | Acidic (%) | Main (%) | Basic (%) | Acidic (%) | Main (%) | Basic (%) |
| A | 0 | 16.18 | 54.98 | 28.84 | 16.18 | 54.98 | 28.84 |
| | 1 | 16.05 | 55.35 | 28.60 | 17.79 | 53.91 | 28.30 |
| | 3 | 16.05 | 54.49 | 29.46 | 21.28 | 50.34 | 28.38 |
| | 6 | 16.55 | 54.43 | 29.02 | 27.28 | 46.12 | 26.60 |
| | 12 | 17.20 | 53.60 | 29.20 | NA | NA | NA |
| | | 2-8° C. | | | 25° C. | | |
| Formulation | Time (m) | Acidic (%) | Main (%) | Basic (%) | Acidic (%) | Main (%) | Basic (%) |
| B | 0 | 16.17 | 55.07 | 28.76 | 16.08 | 54.98 | 28.94 |
| | 1 | 16.09 | 55.37 | 28.54 | 17.52 | 54.18 | 28.30 |
| | 3 | 16.02 | 54.57 | 29.42 | 20.30 | 51.11 | 28.58 |
| | 6 | 16.46 | 54.43 | 29.11 | 25.44 | 47.43 | 27.13 |
| | 12 | 16.90 | 53.90 | 29.20 | NA | NA | NA |
| | | 2-8° C. | | | 25° C. | | |
| Formulation | Time (m) | Acidic (%) | Main (%) | Basic (%) | Acidic (%) | Main (%) | Basic (%) |
| C | 0 | 16.13 | 54.88 | 28.99 | 16.08 | 54.98 | 28.94 |
| | 1 | 15.97 | 55.44 | 28.60 | 17.81 | 53.90 | 28.29 |
| | 3 | 16.03 | 54.50 | 29.47 | 21.31 | 50.22 | 28.47 |
| | 6 | 16.47 | 54.37 | 29.15 | 27.53 | 45.85 | 26.62 |
| | 12 | 17.10 | 53.70 | 29.20 | NA | NA | NA |
| | | 2-8° C. | | | 25° C. | | |
| Formulation | Time (m) | Acidic (%) | Main (%) | Basic (%) | Acidic (%) | Main (%) | Basic (%) |
| D | 0 | 16.12 | 54.84 | 29.04 | 16.08 | 54.98 | 28.94 |
| | 1 | 15.99 | 55.43 | 28.58 | 17.47 | 54.15 | 28.39 |
| | 3 | 16.06 | 54.54 | 29.40 | 20.31 | 50.85 | 28.84 |
| | 6 | 16.41 | 54.32 | 29.27 | 25.68 | 47.14 | 27.18 |
| | 12 | 16.70 | 54.10 | 29.20 | NA | NA | NA |
| | | 2-8° C. | | | 25° C. | | |
| Formulation | Time (m) | Acidic (%) | Main (%) | Basic (%) | Acidic (%) | Main (%) | Basic (%) |
| Control | 0 | 16.08 | 54.98 | 28.94 | 16.08 | 54.98 | 28.94 |
| | 1 | 16.01 | 55.27 | 28.71 | 17.14 | 53.99 | 28.87 |
| | 3 | 16.10 | 54.38 | 29.53 | 20.48 | 50.45 | 29.07 |
| | 6 | 16.37 | 54.23 | 29.40 | 26.04 | 46.07 | 27.90 |
| | 12 | 16.90 | 53.20 | 29.60 | NA | NA | NA |

### Size Variant Determination of Formulations A to D

Relative IgG peak (adalimumab having two heavy chains and two light chains) or relative light chain and heavy chain is determined by capillary electrophoresis-sodium dodecyl sulfate (CE-SDS) under reducing or non-reducing conditions, respectively.

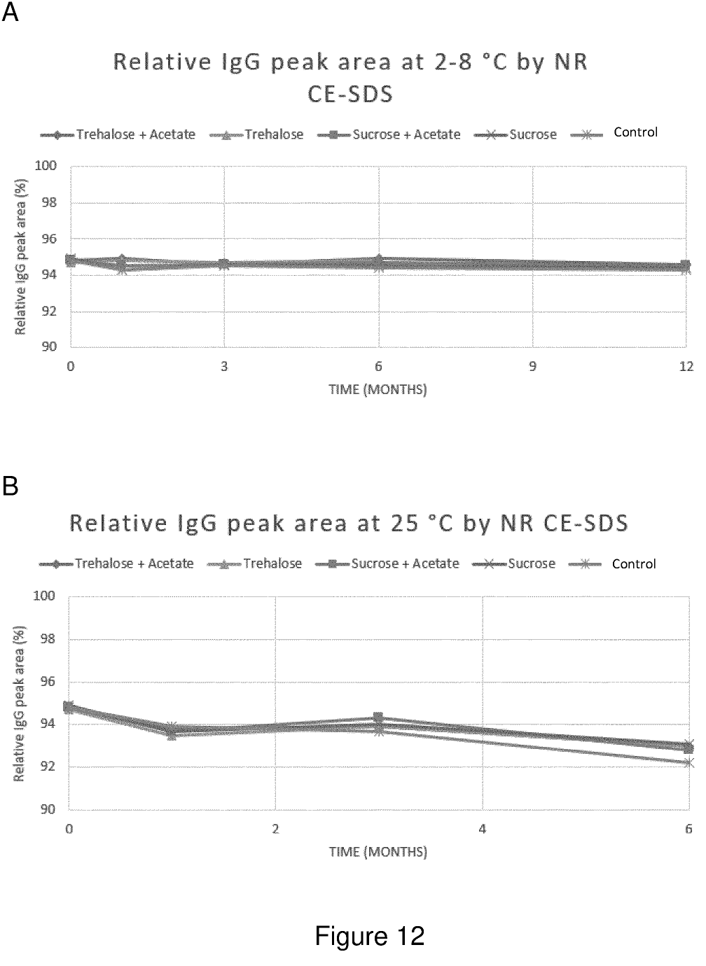

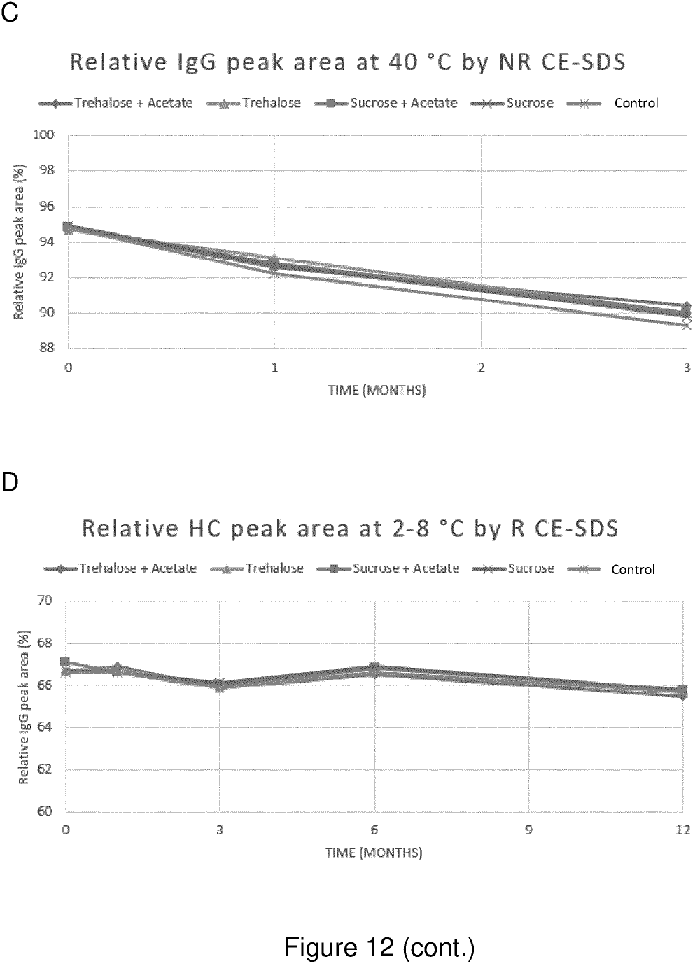

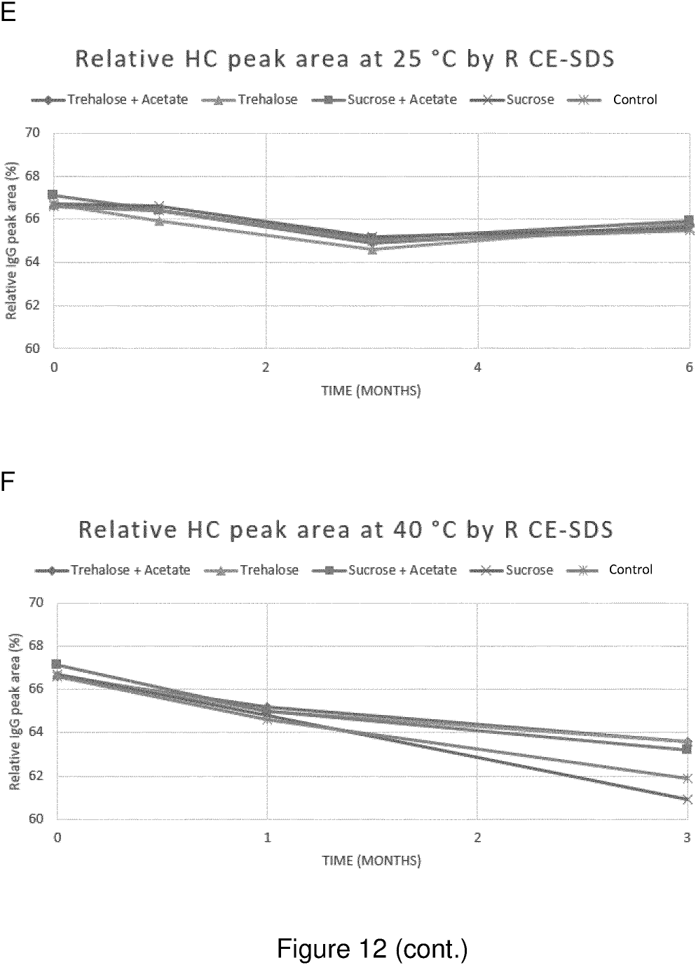

The CE-SDS method is performed using a Maurice CE instrument from Protein Simple. The separation is achieved on a capillary in the Maurice CE-SDS cartridge from Protein Simple. Test samples are prepared by first diluting the mAb solution to 10 mg/mL with a 50 mM phosphate buffer pH 6.0 with 1% SDS. For non reduced conditions the 10 mg/mL sample is treated with a separation mix containing NEM and for reduced conditions the 10 mg/mL sample is treated with a separation mix containing β-mercaptoethanol. Instrument control and data acquisition is performed using Compass for ICE software (Protein Simple) and data handling and reporting is performed using Empower 3 software (Waters). The results of the analyses are shown in Table 10 (Non-Reducing CE-SDS) and Table 11 (Reducing CE-SDS). In Table 10, “IgG” is adalimumab having two heavy chains and two light chains, “HHL” is a variant with two heavy chains and one light chain. In Table 11, “HC” is the heavy chain, “LC” is the light chain, and “DHC” is deglycosylated heavy chain. FIGS. 12A to 12F show the relative IgG peak area for 2° C. to 8° C. storage conditions measured at 0, 1, 3, 6, and 12 months, 25° C. storage conditions measured at 0, 1, 3, and 6 months, and 40° C. storage condition measured 0, 1, and 3 months under non-reducing conditions (NR CE-SDS) and reducing conditions (R CE-SDS).

TABLE 10

Non Reducing CE-SDS

| | | 2-8° C. | | | 25° C. | | | 40° C. | | |
|---|---|---|---|---|---|---|---|---|---|---|
| Form. | Time (m) | NR-IgG (%) | NR -HHL (%) | NR -Other | NR -IgG (%) | NR -HHL (%) | NR -Other | NR IgG (%) | NR-HHL (%) | NR-Other |
| A | 0 | 94.8 | 3.6 | 1.7 | 94.8 | 3.6 | 1.7 | 94.8 | 3.6 | 1.7 |
| | 1 | 94.9 | 3.4 | 1.6 | 93.8 | 3.6 | 2.5 | 92.6 | 3.6 | 3.9 |
| | 3 | 94.6 | 3.8 | 1.7 | 93.9 | 3.9 | 2.1 | 90.4 | 3.9 | 5.7 |
| | 6 | 94.9 | 3.5 | 1.6 | 93 | 3.7 | 3.3 | N.A. | N.A. | N.A. |
| | 12 | 94.6 | 3.8 | 1.7 | N.A. | N.A. | N.A. | N.A. | N.A. | N.A. |
| | | 2-8° C. | | | 25° C. | | | 40° C. | | |
| Form. | Time (m) | NR-IgG (%) | NR -HHL (%) | NR -Other | NR -IgG (%) | NR -HHL (%) | NR -Other | NR IgG (%) | -HHL (%) | -Other |
| B | 0 | 94.7 | 3.7 | 1.6 | 94.7 | 3.7 | 1.6 | 94.7 | 3.7 | 1.6 |
| | 1 | 94.8 | 3.3 | 1.9 | 93.5 | 3.6 | 2.8 | 93.1 | 3.4 | 3.6 |
| | 3 | 94.7 | 3.7 | 1.6 | 93.9 | 4 | 2.1 | 90 | 4 | 6 |
| | 6 | 94.8 | 3.5 | 1.8 | 93 | 3.7 | 3.2 | N.A. | N.A. | N.A. |
| | 12 | 94.6 | 3.7 | 1.7 | N.A. | N.A. | N.A. | N.A. | N.A. | N.A. |
| | | 2-8° C. | | | 25° C. | | | 40° C. | | |
| Form. | Time (m) | NR-IgG (%) | NR -HHL (%) | NR -Other | NR -IgG (%) | NR -HHL (%) | NR -Other | NR IgG (%) | NR-HHL (%) | NR-Other |
| C | 0 | 94.8 | 3.6 | 1.8 | 94.8 | 3.6 | 1.8 | 94.8 | 3.6 | 1.8 |
| | 1 | 94.5 | 3.6 | 1.9 | 93.7 | 3.6 | 2.7 | 92.8 | 3.5 | 3.7 |
| | 3 | 94.6 | 3.7 | 1.6 | 94.3 | 4 | 1.7 | 90 | 4 | 6 |
| | 6 | 94.7 | 3.4 | 2 | 92.8 | 3.6 | 3.6 | N.A. | N.A. | N.A. |
| | 12 | 94.5 | 3.7 | 1.8 | N.A. | N.A. | N.A. | N.A. | N.A. | N.A. |
| | | 2-8° C. | | | 25° C. | | | 40° C. | | |
| Form. | Time (m) | NR-IgG (%) | NR -HHL (%) | NR -Other | NR -IgG (%) | NR -HHL (%) | NR -Other | NR IgG (%) | NR-HHL (%) | NR-Other |
| D | 0 | 94.9 | 3.6 | 1.5 | 94.9 | 3.6 | 1.5 | 94.9 | 3.6 | 1.5 |
| | 1 | 94.3 | 3.4 | 2.2 | 93.7 | 3.7 | 2.6 | 92.7 | 3.5 | 3.7 |
| | 3 | 94.6 | 3.7 | 1.6 | 94 | 3.9 | 1.9 | 89.8 | 3.9 | 6.3 |
| | 6 | 94.5 | 3.4 | 2.1 | 93.1 | 3.6 | 3.3 | N.A. | N.A. | N.A. |
| | 12 | 94.4 | 3.8 | 1.8 | N.A. | N.A. | N.A. | N.A. | N.A. | N.A. |
| | | 2-8° C. | | | 25° C. | | | 40° C. | | |
| Form. | Time (m) | NR-IgG (%) | NR -HHL (%) | NR -Other | NR -IgG (%) | NR -HHL (%) | NR -Other | NR IgG (%) | NR-HHL (%) | NR-Other |
| Control | 0 | 94.8 | 3.8 | 1.5 | 94.8 | 3.8 | 1.5 | 94.8 | 3.8 | 1.5 |
| | 1 | 94.3 | 3.5 | 2.3 | 93.9 | 3.6 | 2.4 | 92.2 | 3.6 | 4.2 |
| | 3 | 94.5 | 3.9 | 1.7 | 93.7 | 4.2 | 2 | 89.3 | 4 | 6.7 |
| | 6 | 94.4 | 3.6 | 2.1 | 92.2 | 3.7 | 4.1 | N.A. | N.A. | N.A. |
| | 12 | 94.3 | 3.9 | 1.9 | N.A. | N.A. | N.A. | N.A. | N.A. | N.A. |

TABLE 11

Reducing CE-SDS

| | | 2-8° C. | | | | 25° C. | | | | 40° C. | | | |
|---|---|---|---|---|---|---|---|---|---|---|---|---|---|
| Form. | Time (m) | R-HC (%) | R-LC (%) | R-DHC (%) | R-Other (%) | R-HC (%) | R-LC (%) | R-DHC (%) | R-Other (%) | R-HC (%) | R-LC (%) | R-DHC (%) | R-Other (%) |
| A | 0 | 66.6 | 31.8 | 1.5 | 0.1 | 66.6 | 31.8 | 1.5 | 0.1 | 66.6 | 31.8 | 1.5 | 0.1 |
| | 1 | 66.9 | 31.6 | 1.5 | 0 | 66.4 | 32.1 | 1.5 | 0 | 65.2 | 32.3 | 1.5 | 1.1 |
| | 3 | 65.9 | 31.8 | 1.6 | 0.7 | 64.9 | 32.5 | 1.7 | 0.9 | 63.6 | 33.1 | 1.6 | 1.7 |
| | 6 | 66.5 | 31.6 | 1.7 | 0.2 | 65.7 | 31.6 | 1.6 | 1 | N.A. | N.A. | N.A. | N.A. |
| | 12 | 65.5 | 32.7 | 1.7 | 0 | N.A. | N.A. | N.A. | N.A. | N.A. | N.A. | N.A. | N.A. |
| | | 2-8° C. | | | | 25° C. | | | | 40° C. | | | |
| Form. | Time (m) | R-HC (%) | R-LC (%) | R-DHC (%) | R-Other (%) | R-HC (%) | R-LC (%) | R-DHC (%) | R-Other (%) | R-HC (%) | R-LC (%) | R-DHC (%) | R-Other (%) |
| B | 0 | 66.7 | 31.7 | 1.6 | 0 | 66.7 | 31.7 | 1.6 | 0 | 66.7 | 31.7 | 1.6 | 0 |
| | 1 | 66.8 | 31.7 | 1.4 | 0.1 | 65.9 | 32.2 | 1.5 | 0.4 | 65 | 32.3 | 1.6 | 1.1 |
| | 3 | 65.9 | 31.9 | 1.6 | 0.7 | 64.6 | 32.1 | 1.9 | 1.4 | 63.6 | 32.5 | 1.7 | 2.2 |
| | 6 | 66.8 | 31.3 | 1.7 | 0.2 | 65.8 | 31.8 | 1.6 | 0.9 | N.A. | N.A. | N.A. | N.A. |
| | 12 | 65.7 | 32.6 | 1.7 | 0 | N.A. | N.A. | N.A. | N.A. | N.A. | N.A. | N.A. | N.A. |
| | | 2-8° C. | | | | 25° C. | | | | 40° C. | | | |
| Form. | Time (m) | R-HC (%) | R-LC (%) | R-DHC (%) | R-Other (%) | R-HC (%) | R-LC (%) | R-DHC (%) | R-Other (%) | R-HC (%) | R-LC (%) | R-DHC (%) | R-Other (%) |
| C | 0 | 67.1 | 31.5 | 1.5 | 0 | 67.1 | 31.5 | 1.5 | 0 | 67.1 | 31.5 | 1.5 | 0 |
| | 1 | 66.6 | 31.9 | 1.5 | 0 | 66.4 | 32.1 | 1.5 | 0 | 65 | 32.5 | 1.5 | 1.1 |
| | 3 | 66 | 31.9 | 1.6 | 0.5 | 65.1 | 32.3 | 1.6 | 0.9 | 63.2 | 32.7 | 1.7 | 2.5 |
| | 6 | 66.8 | 31.4 | 1.6 | 0.2 | 65.9 | 31.6 | 1.6 | 0.9 | N.A. | N.A. | N.A. | N.A. |
| | 12 | 65.8 | 32.5 | 1.7 | 0 | N.A. | N.A. | N.A. | N.A. | N.A. | N.A. | N.A. | N.A. |
| | | 2-8° C. | | | | 25° C. | | | | 40° C. | | | |
| Form. | Time (m) | R-HC (%) | R-LC (%) | R-DHC (%) | R-Other (%) | R-HC (%) | R-LC (%) | R-DHC (%) | R-Other (%) | R-HC (%) | R-LC (%) | R-DHC (%) | R-Other (%) |
| D | 0 | 66.7 | 31.7 | 1.5 | 0 | 66.7 | 31.7 | 1.5 | 0 | 66.7 | 31.7 | 1.5 | 0 |
| | 1 | 66.6 | 31.9 | 1.5 | 0 | 66.6 | 31.9 | 1.5 | 0 | 64.8 | 32.7 | 1.5 | 1.1 |
| | 3 | 66.1 | 32 | 1.6 | 0.3 | 65.2 | 32.4 | 1.6 | 0.7 | 60.9 | 31.8 | 2 | 5.4 |
| | 6 | 66.9 | 31.4 | 1.6 | 0.1 | 65.6 | 31.6 | 1.7 | 1 | N.A. | N.A. | N.A. | N.A. |
| | 12 | 65.8 | 32.5 | 1.7 | 0.0 | N.A. | N.A. | N.A. | N.A. | N.A. | N.A. | N.A. | N.A. |
| | | 2-8° C. | | | | 25° C. | | | | 40° C. | | | |
| Form. | Time (m) | R-HC (%) | R-LC (%) | R-DHC (%) | R-Other (%) | R-HC (%) | R-LC (%) | R-DHC (%) | R-Other (%) | R-HC (%) | R-LC (%) | R-DHC (%) | R-Other (%) |
| Control | 0 | 66.6 | 31.7 | 1.6 | 0.2 | 66.6 | 31.7 | 1.6 | 0.2 | 66.6 | 31.7 | 1.6 | 0.2 |
| | 1 | 66.6 | 31.9 | 1.6 | 0.0 | 66.4 | 32.1 | 1.4 | 0.1 | 64.6 | 32.4 | 1.4 | 1.6 |
| | 3 | 65.9 | 32.2 | 1.6 | 0.3 | 65 | 32.6 | 1.6 | 0.8 | 61.9 | 32.5 | 1.6 | 4 |
| | 6 | 66.6 | 31.6 | 1.6 | 0.2 | 65.5 | 31.7 | 1.6 | 1.3 | N.A. | N.A. | N.A. | N.A. |
| | 12 | 65.7 | 32.5 | 1.7 | 0.0 | N.A. | N.A. | N.A. | N.A. | N.A. | N.A. | N.A. | N.A. |

### Visual Inspection of Formulations A to D

The samples were inspected to visual particles (Table 12).

The samples were inspected using a Seidenader V90-T visual inspection machine, where the samples were rotated in front of highly concentrated light beams to maximize the reflect of visible particles.

TABLE 12

2-8° C.

| Formulation | Description | Time (weeks) | | | | |
|---|---|---|---|---|---|---|
| | | 0 | 4 | 12 | 26 | 52 |
| A | Trehalose + Acetate | | | 2 | | |
| B | Trehalose | | | | | |
| C | Sucrose + Acetate | | | | 1 | |
| D | Sucrose | | | | | |
| Control | Control | | | 1 | | |

25° C.

| Formulation | Description | Time (weeks | | | |
|---|---|---|---|---|---|
| | | 0 | 4 | 12 | 26 |
| A | Trehalose + Acetate | | | | |
| B | Trehalose | | | | |
| C | Sucrose + Acetate | | | | |
| D | Sucrose | | | 1 | |
| Control | Control | | | 3 | |

40° C.

| Formulation | Description | Time (weeks) | | |
|---|---|---|---|---|
| | | 0 | 4 | 12 |
| A | Trehalose + Acetate | | | |
| B | Trehalose | | | |
| C | Sucrose + Acetate | | | 1 |
| D | Sucrose | | | |
| Control | Control | | | 3 |

Code: No visible particles Intrinsic Extrinsic not measured (52 Weeks)
*Note: all visible particles at Time 12 weeks had the same characteristics. The particles are easy to identify, it is possible that they are silicone, since they are transparent and move upwards in syringe. The same particles have also detected in some buffer syringes, which confirms that these are not protein particles.

No samples were indicated as “extrinsic”.

### Osmolality and pH of Formulations A to D

The osmolality (mOsm/kg) was measured.

The osmolality measurements are preformed using Osmomat 3000 (Gonotech), a freezing point osmometer. A 50 µL sample is measured undiluted.

The pH was also determined.

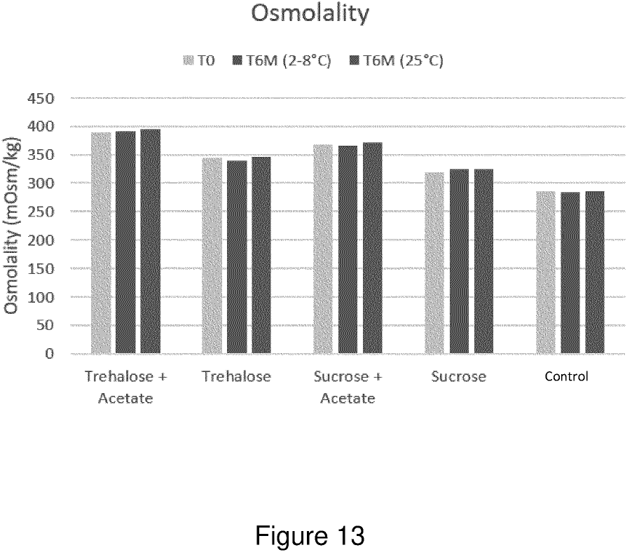

The osmolality and pH are shown in Table 13 and the osmolality is shown in FIG. 13.

TABLE 13

| Formulation | Osmolality (mOsm/kg) | | | | pH | | | | OD280 (mg/ml ) |
|---|---|---|---|---|---|---|---|---|---|
| | T0 | T6M (2-8° C.) | T6M (25° C. ) | T0 | | T6M (2-8° C.) | T6M (25° C. ) | T12M (2-8° C.) | T0 |
| A (Trehalose + Acetate) | 390 | 391 | 395 | 5.4 | | 5.6 | 5.5 | 5.5 | 106.0 |
| B (Trehalose) | 344 | 340 | 346 | 5.43 | | 5.5 | 5.5 | 5.5 | 106.2 |
| C (Sucrose + Acetate) | 367 | 366 | 371 | 5.4 | | 5.5 | 5.5 | 5.5 | 101.6 |
| D (Sucrose) | 319 | 324 | 325 | 5.44 | | 5.5 | 5.5 | 5.5 | 100.2 |
| Control | 285 | 284 | 285 | 5.1 | | 5.2 | 5.2 | 5.1 | 109.8 |

### Biological Activity (Binding to TNFA)

The biological activity of adalimumab (binding to TNFa) was determined for the formulations under the storage conditions.

The binding of adalimumab to TNF-α is detected on a gold-coated microfluidic flow sensor chip comprised of four separate flow cells using surface plasmon resonance technology (Biacore T200, GE Healthcare). Adalimumab is captured via Protein A on the gold layer surface in every flow cells 2-4 of the sensor chip. The first flow cell is left blank and used as a reference flow cell. The ligand, TNF-α, in 5 concentrations is injected over all four flow cells of the sensor surface in parallel. The injections are matched by reference injections, i.e. buffer containing no ligand. Both, buffer injections and the reference flow cell signals are subtracted from the actual signal (double referencing). Captured adalimumab on the chip surface binds specifically to TNF-α. The binding and the dissociation are detected and quantified by means of the change of the surface-plasmon resonance signal caused by the change in refractive index on the gold layer. The strength of the signal detected on the surface is dependent on the concentration of the injected ligand. The time courses of signal changes caused by binding and dissociation of TNF-α are recorded as sensorgrams and used to determine the kinetic parameters ka, kd and KD. For evaluation of relative binding the KD value of the sample is normalized with the KD value of the reference batch.

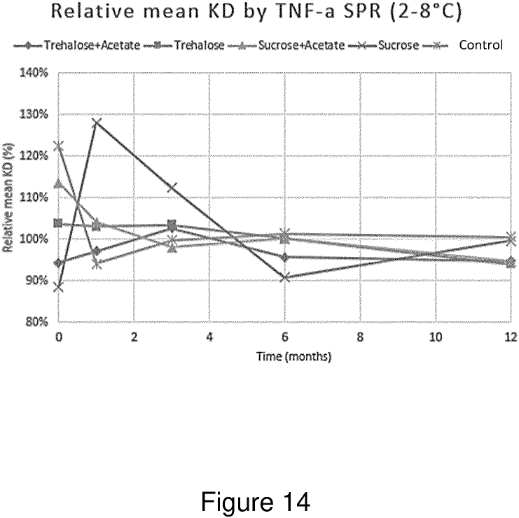

The relative mean KD by TNF-alpha as measured by Surface Plasmon Resonance (SPR) is reported in Table 14 and FIG. 14.

TABLE 14

| A: Trehalose + Acetate | | 2-8° C. | | | 25° C. | | |
|---|---|---|---|---|---|---|---|
| Formulation | Time (m) | Relative KD (%) | SD | CV | Relative KD (%) | SD | CV |
| A | 0 | 94.3% | 1.9% | 2.0% | 94.3% | 1.9% | 2.0% |
| | 1 | 97.0% | 6.5% | 6.7% | 98.0% | 5.0% | 5.1% |
| | 3 | 102.6% | 3.5% | 3.4% | NA | NA | NA |
| | 6 | 95.5% | 0.7% | 0.8% | 103.5% | 4.5% | 4.4% |
| | 12 | 94.6% | N/A | N/A | N/A | N/A | N/A |
| B: Trehalose | | 2-8° C. | | | 25° C. | | |
| Formulation | Time (m) | Relative KD (%) | SD | CV | Relative KD (%) | SD | CV |
| B | 0 | 103.6% | 3.2% | 3.1% | 103.6% | 3.2% | 3.1% |
| | 1 | 103.0% | 7.5% | 7.3% | 102.0% | 5.2% | 5.1% |
| | 3 | 103.4% | 2.1% | 2.1% | NA | NA | NA |
| | 6 | 100.1% | 6.4% | 6.4% | 102.9% | 2.2% | 2.1% |
| | 12 | 93.9% | N/A | N/A | NA | NA | NA |
| C: Sucrose + Acetate | | 2-8° C. | | | 25° C. | | |
| Formulation | Time (m) | Relative KD (%) | SD | CV | Relative KD (%) | SD | CV |
| C | 0 | 113.5% | 2.8% | 2.5% | 113.5% | 2.8% | 2.5% |
| | 1 | 104.0% | 5.9% | 5.7% | 104.0% | 7.0% | 6.8% |
| | 3 | 97.9% | 5.8% | 5.9% | NA | NA | NA |
| | 6 | 100.0% | 5.7% | 5.7% | 101.7% | 0.7% | 0.7% |
| | 12 | 94.5% | N/A | N/A | NA | NA | NA |
| D: Sucrose | | 2-8° C. | | | 25° C. | | |
| Formulation | Time (m) | Relative KD (%) | SD | CV | Relative KD (%) | SD | CV |
| D | 0 | 88.4% | 1.6% | 1.8% | 88.4% | 1.6% | 1.8% |
| | 1 | 128.0% | 7.7% | 6.0% | 105.0% | 6.4% | 6.1% |
| | 3 | 112.3% | 2.2% | 1.9% | NA | NA | NA |
| | 6 | 90.7% | 8.7% | 9.6% | 104.8% | 1.7% | 1.7% |
| | 12 | 99.5% | N/A | N/A | NA | NA | NA |
| Control | | 2-8° C. | | | 25° C. | | |
| Formulation | Time (m) | Relative KD (%) | SD | CV | Relative KD (%) | SD | CV |
| Control | 0 | 122.3% | 1.3% | 1.0% | 122.3% | 1.3% | 1.0% |
| | 1 | 94.0% | 11.3% | 12.0% | 104.0% | 6.9% | 6.6% |
| | 3 | 99.6% | 4.3% | 4.3% | NA | NA | NA |
| | 6 | 101.2% | 2.9% | 2.9% | 106.2% | 0.6% | 0.6% |
| | 12 | 100.5% | N/A | N/A | NA | NA | NA |

### Biological Activity (FCYRIIIA Binding)

The biological activity of Adalimumab (binding to FcγRIIIa) was determined for the formulations under the storage conditions.

The binding of adalimumab to FcγRIIIa is detected on a gold-coated microfluidic flow sensor chip comprised of four separate flow cells using surface plasmon resonance technology (Biacore T200, GE Healthcare). The ligand, FcyRIIIa, is covalently immobilized on a functionalized gold layer surface in flow cells 2-4 of the sensor chip via amine coupling chemistry. While flow cell 1 is blank immobilized with ethanolamine and used as a reference flow cell. Adalimumab is injected over all four flow cells of the sensor surface sequentially. The injections are matched by reference injections, i.e. buffer containing no adalimumab. Both, buffer injections and the reference flow cell signals are subtracted from the actual signal (double referencing). The Fc region of adalimumab binds to the immobilized FcγRIIIa on the chip surface in a glycosylation specific manner. The binding and the dissociation of adalimumab are detected and quantified by means of the change of the surface-plasmon resonance signal caused by the change in refractive index on the gold layer. The strength of the signal detected on the surface is dependent on the concentration of the injected antibody. The signal changes caused by binding and dissociation of adalimumab are recorded as sensorgrams and used to determine the steady state affinity parameter KD. For evaluation of relative binding the KD value of the sample is normalized with the KD value of the reference batch.

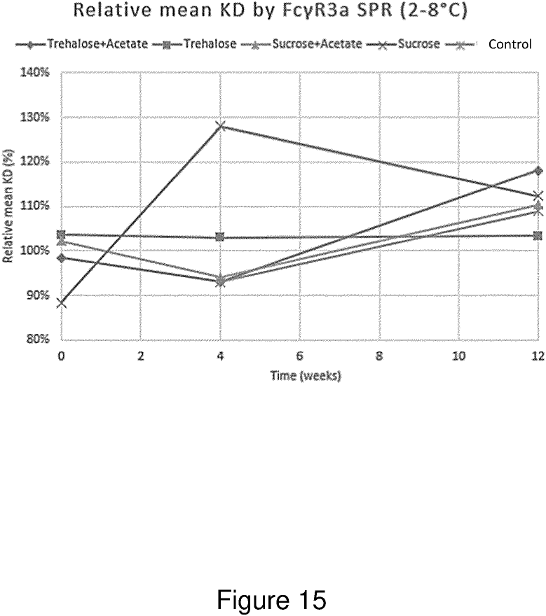

The relative mean KD by FcγRIIIa as measured by Surface Plasmon Resonance (SPR) is reported in Table 15 and FIG. 15.

TABLE 15

| A: Trehalose + Acetate | | 2-8° C. | | | 25° C. | | |
|---|---|---|---|---|---|---|---|
| Formulation | Time (w) | Relative KD (%) | SD | CV | Relative KD (%) | SD | CV |
| A | 0 | 98.4% | 2.7% | 2.8% | 98.4% | 2.7% | 2.8% |
| | 4 | 93.0% | 6.0% | 6.5% | 92.0% | 5.4% | 5.9% |
| | 12 | 118.1% | 6.1% | 5.2% | | | |

| B: Trehalose | | 2-8° C. | | | 25° C. | | |
|---|---|---|---|---|---|---|---|
| Formulation | Time (w) | Relative KD (%) | SD | CV | Relative KD (%) | SD | CV |
| B | 0 | 100.9% | 3.0% | 3.0% | 100.9% | 3.0% | 3.0% |
| | 4 | 94.0% | 9.0% | 9.6% | 101.0% | 8.9% | 8.9% |
| | 12 | 109.8% | 5.7% | 5.2% | | | |
| C: Sucrose + Acetate | | 2-8° C. | | | 25° C. | | |
| Formulation | Time (w) | Relative KD (%) | SD | CV | Relative KD (%) | SD | CV |
| C | 0 | 102.2% | 2.1% | 2.0% | 102.2% | 2.1% | 2.0% |
| | 4 | 94.0% | 7.5% | 8.0% | 92.0% | 8.3% | 9.1% |
| | 12 | 110.4% | 5.8% | 5.2% | | | |
| D: Sucrose | | 2-8° C. | | | 25° C. | | |
| Formulation | Time (w) | Relative KD (%) | SD | CV | Relative KD (%) | SD | CV |
| D | 0 | 95.8% | 2.3% | 2.4% | 95.8% | 2.3% | 2.4% |
| | 4 | 123.0% | 10.9% | 8.9% | 100.0% | 7.2% | 7.2% |
| | 12 | 110.3% | 5.1% | 4.6% | | | |

| | | | | | | | |
|---|---|---|---|---|---|---|---|
| D: Control | | 2-8° C. | | | 25° C. | | |
| Formulation | Time (w) | Relative KD (%) | SD | CV | Relative KD (%) | SD | CV |
| Control | 0 | N.A. | 49.6% | 8.6% | N.A. | 49.6% | N.A. |
| | 4 | 93.0% | 10.5% | 11.2% | 88.0% | 8.6% | 9.7% |
| | 12 | 108.9% | 5.1% | 4.7% | | | |
| | | 578.2% | | | 578.2% | | |

*N.A.: T0 value re outlier: emoved -

### Oxidation/Deamidation

The oxidation/deamidation of adalimumab was determined for the formulations under the storage conditions (Tables 16 and 17).

Oxidation and deamidation testing was done by LC-MS using a Bruker micrOTOF mass spectrometer in combination with a Dionex Ultimate3000RSLC nano liquid chromatography system, in the positive mode, with electrospray (ESI) ionization. Spectra were recorded in the mass range between 200-2500 m/z with a spectra rate of 1 Hz. For the analysis a standard method for ESI-MS of peptides or proteins was applied. Peptide separation was achieved on a (column) using a linear gradient from 1% to 35% mobile phase (MP) B over 145 minutes, following 10 minutes of isocratic elution at 1% MP B. MP A was 0.1% formic acid (FA) in water and MP B was 0.1% FA in 90% aqueous acetonitrile (ACN). Flow rate was 8 uL/min and 0.8 µg of digested protein was injected. Sample preparation steps were 1) denaturation and reduction in 6 M guanidine HCl and 100 mM Tris at pH 7.8, with dithiothreitol (DTT) as the reducing agent and methionine as an antioxidant, 2) Cysteine alkylation with iodoacetamide, 3) Buffer exchange to 100 mM Tris, 0.8 M Urea, pH 7.0 by dialysis (Scienova; Xpress Micro Dialyser, 12-14 kDa), 4) Digestion with Trypsin in presence of CaCl₂ to enhance Trypsin activity and methionine as antioxidant and 5) Dilution with aqueous FA to 0.2 mg/mL protein and 2% (v/v) FA concentration. All data were processed with the software package Data Analysis (version 4.4, Bruker Daltonik).

TABLE 16

| | | Deamidation (%) | | | | | |
|---|---|---|---|---|---|---|---|
| Formulation | Time (m) | HT8 | HT13-14 | HT27 | HT37 | HT38 | LT14 |
| A | 0 | 1.7 | 1.8 | 4.1 | 1.1 | 3.2 | 0.7 |
| | 3 | 0.6 | 3.4 | 2.8 | 1.0 | 4.3 | 1.9 |
| | 12 | 1.2 | 0.0 | 2.8 | 0.6 | 2.1 | 0.7 |
| | | Deamidation (%) | | | | | |
| | Time (m) | HT8 | HT13-14 | HT27 | HT37 | HT38 | LT14 |
| B | 0 | 1.7 | 2.9 | 4.3 | 1.0 | 3.3 | 0.7 |
| | 3 | 0.7 | 3.4 | 3.3 | 1.1 | 3.1 | 1.7 |
| | 12 | 0.9 | 0.0 | 2.2 | 0.7 | 1.6 | 0.8 |
| | | Deamidation (%) | | | | | |
| | Time (m) | HT8 | HT13-14 | HT27 | HT37 | HT38 | LT14 |
| C | 0 | 1.8 | 3.3 | 3.7 | 1.1 | 2.7 | 0.7 |
| | 3 | 0.6 | 4.2 | 3.8 | 1.0 | 3.8 | 1.8 |
| | 12 | 1.0 | 0.0 | 2.6 | 0.7 | 1.8 | 0.9 |
| | | Deamidation (%) | | | | | |
| | Time (m) | HT8 | HT13-14 | HT27 | HT37 | HT38 | LT14 |
| D | 0 | 1.9 | 3.1 | 4.0 | 1.1 | 3.0 | 0.6 |
| | 3 | 0.8 | 3.7 | 3.7 | 1.2 | 4.0 | 1.6 |
| | 12 | 1.0 | 0.0 | 2.4 | 0.6 | 2.1 | 0.8 |
| | | Deamidation (%) | | | | | |
| | Time (m) | HT8 | HT13-14 | HT27 | HT37 | HT38 | LT14 |
| Control | 0 | 1.6 | 3.7 | 3.7 | 1.1 | 3.1 | 0.7 |
| | 3 | 0.8 | 4.4 | 3.2 | 1.1 | 2.9 | 1.4 |
| | 12 | 1.5 | 0.0 | 2.9 | 0.7 | 2.2 | 0.6 |

TABLE 17

| | | Oxidation (%) | | | | |
|---|---|---|---|---|---|---|
| Formulation | Time (m) | HT2/3 | HT8 | HT21 | HT42 | LT1 |
| A | 0 | 1.3 | 0.7 | 7.2 | 3.6 | 0.9 |
| | 3 | 0.6 | 0.4 | 5.4 | 2.7 | 0.5 |
| | 12 | 2.4 | 0.5 | 6.9 | 0.0 | 1.0 |
| | | Oxidation (%) | | | | |
| | Time (m) | HT2/3 | HT8 | HT21 | HT42 | LT1 |
| B | 0 | 1.3 | 0.7 | 7.7 | 3.5 | 0.9 |
| | 1 | 0.4 | 0.4 | 5.1 | 2.4 | 0.5 |
| | 3 | 2.0 | 0.5 | 7.4 | 0.0 | 0.6 |
| | | Oxidation (%) | | | | |
| | Time (m) | HT2/3 | HT8 | HT21 | HT42 | LT1 |
| C | 0 | 1.3 | 0.8 | 8.2 | 3.7 | 1.0 |
| | 1 | 0.5 | 0.4 | 4.7 | 2.5 | 0.5 |
| | 3 | 2.0 | 0.4 | 8.0 | 0.0 | 0.6 |
| | | Oxidation (%) | | | | |
| | Time (m) | HT2/3 | HT8 | HT21 | HT42 | LT1 |
| D | 0 | 1.1 | 0.6 | 7.4 | 3.4 | 0.9 |
| | 1 | 0.5 | 0.4 | 4.7 | 2.2 | 0.5 |
| | 3 | 2.1 | 0.3 | 7.0 | 0.0 | 0.9 |
| | | Oxidation (%) | | | | |
| | Time (m) | HT2/3 | HT8 | HT21 | HT42 | LT1 |
| Control | 0 | 1.2 | 0.6 | 7.2 | 3.2 | 0.8 |
| | 1 | 0.4 | 0.3 | 5.7 | 1.8 | 0.5 |
| | 3 | 1.9 | 0.4 | 6.9 | 0.0 | 0.9 |

The sequences of the digestion peptides are as follows in Table 18.

TABLE 18

| | |
|---|---|
| Code | Sequence |
| HT2 | SLR₁₉ |
| HT3 | LSCAASGFTFDDYAMHW VR₃₈ |
| HT8 | NSLYLQMNSLR₈₇ |
| HT13-14 | DYFPEPVTVSWNSGALTSGVHTFPAVLQSSGLYSLSSVVTVPSSSLGTQMCNVNHKPSNTK₂₁₄ |
| HT21 | DTLMISR₂₅₉ |
| HT27 | VVSVLTVLHQDWLNGK3₂₁ |
| HT37 | NQVSLTCLVK₃₇₄ |
| HT38 | GFYPSDIAVEWESNGQPENNYK₃₉₆ |
| HT42 | WQQGNVFSCSVMfIEALHNHYTQK₄₄₃ |
| LT1 | DIQMTQSPSSLSASVGDR₁₈ |
| LT14 | SGTASVVCLLNNFYPR₁₄₂ |

1. A stable, aqueous formulation comprising:
(a) about 90 mg/ml to about 125 mg/ml anti-TNFα antibody;
(b) about 200 mM to about 275 mM trehalose or about 200 mM to about 275 mM sucrose;
(c) about 0.05% (w/v) to about 0.15% (w/v) nonionic surfactant; and
(d) about 25 mM or less acetate buffer or about 25 mM or less succinate buffer,
wherein the formulation is essentially free of ionic tonicity adjusting agents; wherein the formulation is essentially free of amino acid stabilizer; wherein the osmolality of the formulation is about 240 mOsm/kg to about 420 mOsm/kg; and wherein the formulation is about pH 5.0 to about pH 6.0.

2. The formulation of claim 1, wherein the anti-TNFα antibody is adalimumab.

3. The formulation of claim 2, comprising about 100 mg/ml adalimumab, about 250 mM trehalose, and about 0.1% polysorbate 20, or
comprising about 100 mg/ml adalimumab, about 250 mM sucrose, and about 0.1% polysorbate 20.

4. The formulation of claim 1, comprising about 20 mM acetate buffer or comprising about 20 mM succinate buffer.

5. The formulation of claim 1, wherein the formulation is essentially free of ionic excipients.

6. The formulation of claim 1, wherein the formulation meets one or more of the following criteria:
(a) stable under long term storage, wherein long term storage is about 3 months at about 2° C. to about 8° C.;
(b) stable under long term storage, wherein long term storage is about 6 months at about 2° C. to about 8° C.;
(c) stable under long term storage, wherein long term storage is about 12 months at about 2° C. to about 8° C.; and/or
(d) stable under room temperature storage, wherein room temperature storage is about 14 days.

7. The formulation of claim 1, wherein stability is determined by one or more of the following criteria:
(a) less or about equal increase in aggregation when stored at about 2° C. to about 8° C. for at least about 12 months;
(b) less or about equal increase in aggregation when stored at about 25° C. for at least 1 month;
(c) less or about equal increase in relative percent acid species when stored at about 2° C. to about 8° C. for at least 12 months; and/or
(d) less or about equal increase in relative percent acid species when stored at about 25° C. for at least 1 month;
wherein the formulation is compared to a control; and wherein said control comprises adalimumab at about the same concentration of the formulation.

8. The formulation of claim 1, wherein stability is determined by one or more of the following criteria:
(a) the relative percentage of monomer peak for adalimumab is not less than about 98% after storing the formulation at about 2° C. to about 8° C. for at least 6 months, wherein said relative percentage of monomer peak is determined by SEC-HPLC;
(b) the relative percentage of monomer peak for adalimumab is not less than about 98% after storing the formulation at about 25° C. for at least 1 month, wherein said relative percentage of monomer peak is determined by SEC-HPLC;
(c) the relative acidic species peak for adalimumab is not more than about 25% after storing the formulation at about 2° C. to about 8° C. for at least 12 months; wherein said relative acidic species peak is determined by CEX-HPLC; and/or
(d) the relative acidic species peak for adalimumab is not more than about 25% after storing the formulation at about 25° C. for at least 3 months; wherein said relative acidic species peak is determined by CEX-HPLC.

9. An aqueous formulation comprising
(a) about 100 mg/ml adalimumab;
(b) about 250 mM trehalose, or about 250 nM sucrose;
(c) about 0.1% (w/v) polysorbate 20; and
(d) about 20 mM acetate buffer;
wherein the formulation is essentially free of ionic tonicity adjusting agents; wherein the formulation is essentially free of amino acid stabilizer; and wherein the formulation is about pH 5 to about pH 6.

10. An aqueous formulation consisting essentially of
(a) about 100 mg/ml adalimumab;
(b) about 250 mM trehalose, or about 250 mM sucrose;
(c) about 0.1% (w/v) polysorbate 20; and
(d) about 20 mM acetate buffer;
wherein the formulation is about pH 5 to about pH 6.

11. An aqueous formulation comprising
(a) about 100 mg/ml adalimumab;
(b) about 250 mM trehalose or about 250 mM sucrose; and
(c) about 0.1% (w/v) polysorbate 20;
wherein the formulation is essentially free of ionic excipients; and wherein the formulation is about pH 5 to about pH 6.

12. An aqueous formulation consisting essentially of
(a) about 100 mg/ml adalimumab;
(b) about 250 mM trehalose or about 250 mM sucrose; and
(c) about 0.1% (w/v) polysorbate 20;
wherein the formulation is about pH 5 to about pH 6.

13. The formulation of
claim 1, wherein the formulation has been determined to be stable after exposure to one or more of the following stress conditions:
(a) about 25° C. for about 3 months;
(b) about 2 to about 8° C. for about 3 months; and/or
(c) about 40° C. for about 3 months.

14. The formulation of claim 13, wherein stability is determined by reference to a predefined level of perturbation, wherein the predefined level of perturbation is one or more of:
(a) a relative monomer peak area of adalimumab not less than about 98% as assessed by SEC-HPLC;
(b) a relative acidic species peak area of adalimumab not more than about 25% as assessed by CEX-HPLC;
(c) a relative “IgG” peak (intact adalimumab having two heavy chains and two light chains) not less than about 90% as assessed by CE-SDS (non-reducing);
(d) a relative heavy chain (HC) peak of about 60% to about 72% and/or a relative light chain (LC) peak of about 30% to about 36% as assessed by CD-SDS (reducing); and/or
(e) not more than about 6000 particles of size equal to or greater than about 10 µm and/or not more than about 600 particles of size equal to or greater than about 25 µm are detected as assessed by particle count light obscuration.

15. The formulation of claim 14, wherein stability is determined by reference to a control, wherein said control formulation comprises about 100 mg/ml adalimumab, 230 mM mannitol, 0.1% (w/v) Polysorbate 80, and has a pH of about 5.2; and wherein said control is exposed to the same one or more stress conditions as the formulation .

16. The formulation of claim 1, wherein the pH of the formulation is about pH 5.5.

17. A method of formulating a formulation of claim 1, comprising:
(1) exchanging a first solution of an anti-TNFα antibody into a second solution comprising:
(a) about 200 mM to about 275 mM trehalose or about 200 mM to about 275 mM sucrose; and
(b) about 25 mM or less acetate buffer or about 25 mM or less succinate buffer;
wherein the second solution is essentially free of ionic tonicity-adjusting agents; wherein the second solution is essentially free of amino acid stabilizer; and wherein the second formulation has a pH of about 5.0 to about 6.0, and wherein the concentration of the anti-TNFα antibody after exchange is about 90 mg/ml to about 125 mg/ml; and
(2) diluting the solution of step (1) with a nonionic surfactant to obtain a concentration of about 0.05% (w/v) to about 0.15% (w/v) nonionic surfactant.

18. A method of claim 17, wherein the second solution comprises:
about 200 mM to about 275 mM trehalose or about 200 mM to about 275 mM sucrose; and
wherein the second solution is essentially free of ionic excipients.

19. The method of claim 17, wherein the anti-TNFα antibody is adalimumab.

20. The formulation obtainable by the method of claim 17.

21. The formulation of claim 1, wherein said formulation is in the form of a single-dose prefilled injection pen, single-dose prefilled syringe, or single-dose prefilled vial, or the formulation is in the form of a single-use bag.


SEQUENCE LISTING (12 sequences)

SEQ ID NO: 1 - protein, 214 aa, Artificial Sequence
  Light Chain Sequence
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 451 aa, Artificial Sequence
  heavy chain sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 19 aa, Artificial Sequence
  sequence table 18
LSCAASGFTFDDYAMHWVR

SEQ ID NO: 4 - protein, 11 aa, Artificial Sequence
  sequence table 18
NSLYLQMNSLR

SEQ ID NO: 5 - protein, 63 aa, Artificial Sequence
  sequence table 18
DYFPEPVTVSWNSGALTSGVHTFPAVLQSSGLYSLSSVVTVPSSSLGTQTYICNVNHKPSNTK

SEQ ID NO: 6 - protein, 7 aa, Artificial Sequence
  sequence table 18
DTLMISR

SEQ ID NO: 7 - protein, 16 aa, Artificial Sequence
  sequence table 18
VVSVLTVLHQDWLNGK

SEQ ID NO: 8 - protein, 10 aa, Artificial Sequence
  sequence table 18
NQVSLTCLVK

SEQ ID NO: 9 - protein, 22 aa, Artificial Sequence
  sequence table 18
GFYPSDIAVEWESNGQPENNYK

SEQ ID NO: 10 - protein, 23 aa, Artificial Sequence
  sequence table 18
WQQGNVFSCSVMHEALHNHYTQK

SEQ ID NO: 11 - protein, 18 aa, Artificial Sequence
  sequence table 18
DIQMTQSPSSLSASVGDR

SEQ ID NO: 12 - protein, 16 aa, Artificial Sequence
  sequence table 18
SGTASVVCLLNNFYPR